(19) United States
(12) Patent Application Publication
(10) Pub. No.: US 2025/0099239 A1
(43) Pub. Date: Mar. 27, 2025

(54) PROSTHETIC VALVE WITH SUPPORT ARMS

(71) Applicant: EDWARDS LIFESCIENCES CORPORATION, Irvine, CA (US)

(72) Inventors: Anatoly Dvorsky, Haifa (IL); Eran Grosu, Kfar-Saba (IL); Ilan Leshecz, Hadera (IL); Michael Bukin, Pardes Hanna (IL); Nikolai Gurovich, Hadera (IL); Assaf Sharon, Tel Aviv (IL); David Maimon, Atlit (IL)

(73) Assignee: EDWARDS LIFESCIENCES CORPORATION, Irvine, CA (US)

(21) Appl. No.: 18/969,878

(22) Filed: Dec. 5, 2024

Related U.S. Application Data
(63) Continuation of application No. PCT/US2023/025492, filed on Jun. 16, 2023.
(60) Provisional application No. 63/355,485, filed on Jun. 24, 2022.

(51) Int. Cl. A61F 2/24

(52) U.S. Cl. CPC A61F 2/2418; A61F 2210/0057; A61F 2230/0069

(57) ABSTRACT

Prosthetic valves that include support arms extending from support posts thereof, and methods for utilizing such prosthetic valves, are disclosed herein. As one example, a prosthetic valve can include an annular frame movable between a radially compressed state and a radially expanded state, and at least one support arm. The frame can include a plurality of support posts, each extending between a post inflow end and an opposite post outflow end. The support arm can extend from a corresponding support post, and terminate at a free-ended tip which is biased radially away from the corresponding support post.

### CROSS-REFERENCE TO RELATED APPLICATIONS

This application is a continuation of International Application No. PCT/US2023/025492, filed Jun. 16, 2023, which claims the benefit of U.S. Provisional Application No. 63/355,485, filed Jun. 24, 2022, which is incorporated by reference herein.

### FIELD

The present disclosure relates to mechanically expandable prosthetic valves that include support arms extending from support posts thereof.

### BACKGROUND

Native heart valves, such as the aortic, pulmonary and mitral valves, function to assure adequate directional flow from and to the heart, and between the heart's chambers, to supply blood to the whole cardiovascular system. Various valvular diseases can render the valves ineffective and require replacement with artificial valves. Surgical procedures can be performed to repair or replace a heart valve. Surgeries are prone to an abundance of clinical complications, hence alternative less invasive techniques of delivering a prosthetic valve over a catheter and implanting it over the native malfunctioning valve, have been developed over the years.

Different types of prosthetic valves are known to date, including balloon expandable valve, self-expandable valves and mechanically-expandable valves. Different methods of delivery and implantation are also known, and may vary according to the site of implantation and the type of prosthetic valve. One exemplary technique includes utilization of a delivery assembly for delivering a prosthetic valve in a crimped state, from an incision which can be located at the patient's femoral or iliac artery, toward the native malfunctioning valve. Once the prosthetic valve is properly positioned at the desired site of implantation, it can be expanded against the surrounding anatomy, such as an annulus of a native valve, and the delivery assembly can be retrieved thereafter.

One of the challenges associated with the above-mentioned procedures relates to the ability of the prosthetic valves to be adequately secured relative to the native annulus, in an atraumatic manner. This may be of particular importance in specific pathologies, such as aortic insufficiency or aortic regurgitation, in which the native anatomy may lack calcified deposits that could otherwise support prosthetic valves configured to exert outwardly directed radial forces to retain them in position. Thus, a need exists for improving fixation of prosthetic valve implanted within native anatomical orifices.

### SUMMARY

The present disclosure is directed toward mechanically expandable prosthetic valves that include at least one support arm extending from a vertical support post thereof, configured to resist undesirable axial displacement of the prosthetic valve, relative to the native annulus, after implantation.

According to some aspects of the disclosure, a prosthetic valve comprises an annular frame movable between a radially compressed state and a radially expanded state. The frame comprises a plurality of support posts, a plurality of angled struts extending circumferentially between and interconnected with the support posts, a valvular structure mounted within the frame and comprising a plurality of leaflets configured to regulate flow through the prosthetic valve, and at least one support arm extending at least one of the support posts.

In some aspects, the at least one support arm extends from the post outflow end or the post inflow end of at least one of the support posts.

In some aspects, the support arm terminates at a free-ended tip which is biased radially away from the corresponding support post.

In some aspects, the width of the support post from which the support arm extends is greater than the width of any of the angled struts.

In some aspects, each of the plurality of support posts extends between a post inflow end and an opposite post outflow end.

In some aspects, the plurality of support posts includes a plurality of commissure support posts, wherein each commissure support post includes a commissure window and a plurality of non-commissural support posts, wherein each non-commissural support post is devoid of a commissure window.

In some aspects, the support arm is circumferentially deflected relative to the frame, such that the tip is circumferentially offset from the corresponding support post.

In some aspects, the support arm is twisted.

In some aspects, the support arm comprises a first arm portion extending from a base of the support arm at the end of the corresponding support posts, and a second arm portion continuously extending from the first arm portion to the tip.

In some aspects, the plurality of support arms comprises at least one outflow support arm extending from the post outflow end of at least one of the support posts, and at least one inflow support arm extending from the post inflow end of at least one of the support posts.

In some aspects, the at least one outflow support arm comprises a plurality of outflow support arms.

In some aspects, the at least one inflow support arm comprises a plurality of inflow support arms.

In some aspects, the prosthetic valve further comprises an outer skirt mounted on the frame.

In some aspects, the skirt further comprises a sealing ring extending radially away from the frame.

In some aspects, the sealing ring is compressible.

According to some aspects of the disclosure, a prosthetic valve comprises an annular frame movable between a radially compressed state and a radially expanded state. The frame comprises a plurality of support posts, wherein each support post extends between a post inflow end and an opposite post outflow end, a plurality of angled struts extending circumferentially between and interconnected with the support posts, a valvular structure mounted within the frame and comprising a plurality of leaflets configured to regulate flow through the prosthetic valve, and at least one support arm.

In some aspects, the at least one support arm extends from the opening outflow end or the opening inflow end.

In some aspects, the support arm terminates at a free-ended tip which is biased radially away from the corresponding support post.

In some aspects, the width of the support post from which the support arm extends is greater than the width of any of the angled struts.

In some aspects, at least one of the support posts comprises a post opening extending between an opening outflow end which is distal to the post outflow end, and an opening inflow end which is proximal to the post inflow end.

In some aspects, the plurality of support posts comprises a plurality of commissure support posts, wherein each commissure support post comprises a commissure window and a plurality of non-commissural support posts, wherein each non-commissural support post is devoid of a commissure window.

In some aspects, the support arm is integrally formed with the corresponding support post it extends from.

In some aspects, the at least one support arm comprises a plurality of support arms.

In some aspects, the plurality of support arms comprises inflow support arms extending from the opening inflow ends of corresponding post openings of the support posts.

In some aspects, the plurality of support arms comprises at least one outflow support arm extending from the opening outflow end of the post opening of at least one of the non-commissural support posts, and at least one inflow support arm extending from the opening inflow end of the post opening of at least one of the support posts.

According to one aspect of the disclosure, a method of delivering a prosthetic valve comprises advancing the prosthetic valve in a radially compressed state toward a native heart valve, partially expanding the prosthetic valve at a position in which the outflow end of the prosthetic valve is proximal to native leaflets of the native heart valve, angularly orienting the prosthetic valve such that commissures of the prosthetic valve are aligned with native commissures of the native leaflets, and further expanding the prosthetic valve within a native annulus of the native heart valve.

In some aspects, the prosthetic valve comprises an annular frame movable between a radially compressed state and a radially expanded state.

In some aspects, the frame comprises a plurality of vertical posts comprising a plurality of support posts, a plurality of angled struts extending circumferentially between and interconnected with the vertical posts, and at least one support arm extending from at least one of the vertical posts.

In some aspects, each of the plurality of support posts extends between a post inflow end and an opposite post outflow end.

In some aspects, the plurality of support posts comprises a plurality of commissure support posts, wherein each commissure support post comprises a commissure window, and a plurality of non-commissural support posts, wherein each non-commissural support post is devoid of a commissure window.

In some aspects, the support arm terminates at a free-ended tip which is biased radially away from the corresponding vertical post.

In some aspects, the method further comprises distally advancing the prosthetic valve, after partially expanding it and prior to fully expanding it, until the tips contact an abutment surface of the native heart valve.

In some aspects, fully expanding the prosthetic valve comprises clamping native leaflets of the native heart valve between the frame and the at least one support arm.

In some aspects, advancing a prosthetic valve in a radially compressed state comprises retaining the prosthetic valve in a capsule of a delivery apparatus coupled to the prosthetic valve.

In some aspects, the method further comprises moving the capsule axially relative to the prosthetic valve to expose the prosthetic valve from the capsule, prior to partially expanding the prosthetic valve.

According to some aspects of the disclosure, there is provided a prosthetic valve comprising an annular frame movable between a radially compressed state and a radially expanded state, a valvular structure mounted within the frame and comprising a plurality of leaflets configured to regulate flow through the prosthetic valve, and at least one support arm. The frame comprises one or more pairs of actuation posts, a plurality of support posts, a plurality of angled struts extending circumferentially between adjacent actuation posts and support posts and interconnecting the actuation posts and the support posts, and one or more actuators coupled to the actuation posts. Each support post extends between a post inflow end and an opposite post outflow end. The support posts comprise a plurality of commissure support posts and a plurality of non-commissural support posts. Each commissure support post comprises a commissure window. Each non-commissural support post is devoid of a commissure window. The one or more actuators configured to adjust the frame between the radially compressed state and the radially expanded state. The at least one support arm extends from the post outflow end or the post inflow end of at least one of the support posts, and terminates at a free-ended tip which is biased radially away from the corresponding support post.

According to some aspects of the disclosure, there is provided a prosthetic valve comprising an annular frame movable between a radially compressed state and a radially expanded state, a valvular structure mounted within the frame and comprising a plurality of leaflets configured to regulate flow through the prosthetic valve, and at least one support arm. The frame comprises a plurality of support posts, and a plurality of angled struts extending circumferentially between and interconnected with the support posts. Each support post extends between a post inflow end and an opposite post outflow end. The support posts comprise a plurality of commissure support posts and a plurality of non-commissural support posts. Each commissure support post comprises a commissure window. Each non-commissural support post is devoid of a commissure window. The at least one support arm extends from the post outflow end or the post inflow end of at least one of the support posts, and terminates at a free-ended tip which is biased radially away from the corresponding support post. The width of the support post from which the support arm extends is greater than the width of any of the angled struts.

According to some aspects of the disclosure, there is provided a delivery assembly comprising a prosthetic valve and a delivery apparatus. The prosthetic valve comprises an annular frame movable between a radially compressed state and a radially expanded state, a valvular structure mounted within the frame and comprising a plurality of leaflets configured to regulate flow through the prosthetic valve, and at least one support arm. The frame comprises one or more pairs of actuation posts, a plurality of support posts, a plurality of angled struts extending circumferentially between adjacent actuation posts and support posts and interconnecting the actuation posts and the support posts, and one or more actuators coupled to the actuation posts. Each support post extends between a post inflow end and an opposite post outflow end. The support posts comprise a plurality of commissure support posts and a plurality of non-commissural support posts. Each commissure support post comprises a commissure window. Each non-commissural support post is devoid of a commissure window. The one or more actuators configured to adjust the frame between the radially compressed state and the radially expanded state. The at least one support arm extends from the post outflow end or the post inflow end of at least one of the support posts, and terminates at a free-ended tip which is biased radially away from the corresponding support post.

In some examples, the delivery assembly comprises at least one actuator assembly releasably coupled to the actuator and configured to rotate the actuator to adjust the frame between the radially compressed state and the radially expanded state, a handle comprising one or more control mechanisms, and an outer shaft extending from the handle. At least one of the control mechanisms is configured, upon actuation thereof, to rotate the actuator assembly and the actuator of the prosthetic valve to adjust the frame between the radially compressed state and the radially expanded state. The actuator assembly is disposed within the outer shaft.

According to some aspects of the disclosure, there is provided a prosthetic valve comprising an annular frame movable between a radially compressed state and a radially expanded state, a valvular structure mounted within the frame and comprising a plurality of leaflets configured to regulate flow through the prosthetic valve, and at least one support arm. The frame comprises a plurality of vertical posts, a plurality of angled struts, and one or more actuators. The vertical posts comprise one or more pairs of actuation posts, a plurality of support posts, and at least one post opening comprised in at least one of the vertical posts. Each pair of actuation posts comprises an upper post member and a lower post member. Each support post extends between a post inflow end and an opposite post outflow end. The support posts comprise a plurality of commissure support posts and a plurality of non-commissural support posts. Each commissure support post comprises a commissure window. Each non-commissural support post is devoid of a commissure window. The post opening extends between an opening outflow end and an opening inflow end. The angled struts extend circumferentially between adjacent actuation posts and support posts and interconnect the actuation posts and the support posts. The one or more actuators is coupled to the actuation posts and configured to adjust the frame between the radially compressed state and the radially expanded state. The at least one support arm extends from the opening outflow end or the opening inflow end, wherein the support arm terminates at a free-ended tip which is biased radially away from the corresponding vertical post.

According to some aspects of the disclosure, there is provided a prosthetic valve comprising an annular frame movable between a radially compressed state and a radially expanded state, a valvular structure mounted within the frame and comprising a plurality of leaflets configured to regulate flow through the prosthetic valve, and at least one support arm. The frame comprises a plurality of support posts, and a plurality of angled struts extending circumferentially between and interconnected with the support posts. Each support post extends between a post inflow end and an opposite post outflow end. At least one of the support posts comprises a post opening extending between an opening outflow end which is distal to the post outflow end, and an opening inflow end which is proximal to the post inflow end. The support posts comprise a plurality of commissure support posts and a plurality of non-commissural support posts. Each commissure support post comprises a commissure window. Each non-commissural support post is devoid of a commissure window. The at least one support arm extends from the opening outflow end or the opening inflow end, wherein the support arm terminates at a free-ended tip which is biased radially away from the corresponding vertical post. The width of the support post from which the support arm extends is greater than the width of any of the angled struts.

According to some aspects of the disclosure, there is provided a delivery assembly comprising a prosthetic valve and a delivery apparatus. The prosthetic valve comprises an annular frame movable between a radially compressed state and a radially expanded state, a valvular structure mounted within the frame and comprising a plurality of leaflets configured to regulate flow through the prosthetic valve, and at least one support arm. The frame comprises a plurality of support posts, and a plurality of angled struts extending circumferentially between and interconnected with the support posts. Each support post extends between a post inflow end and an opposite post outflow end. At least one of the support posts comprises a post opening extending between an opening outflow end which is distal to the post outflow end, and an opening inflow end which is proximal to the post inflow end. The support posts comprise a plurality of commissure support posts and a plurality of non-commissural support posts. Each commissure support post comprises a commissure window. Each non-commissural support post is devoid of a commissure window. The at least one support arm extends from the opening outflow end or the opening inflow end, wherein the support arm terminates at a free-ended tip which is biased radially away from the corresponding vertical post. The width of the support post from which the support arm extends is greater than the width of any of the angled struts.

In some examples, the delivery assembly comprises at least one actuator assembly releasably coupled to the actuator and configured to rotate the actuator to adjust the frame between the radially compressed state and the radially expanded state, a handle comprising one or more control mechanisms, and an outer shaft extending from the handle. At least one of the control mechanisms is configured, upon actuation thereof, to rotate the actuator assembly and the actuator of the prosthetic valve to adjust the frame between the radially compressed state and the radially expanded state. The actuator assembly is disposed within the outer shaft.

According to some aspects of the disclosure, there is provided a prosthetic assembly comprising a docking station and a prosthetic valve. The prosthetic valve comprises an annular frame movable between a radially compressed state and a radially expanded state, a valvular structure mounted within the frame and comprising a plurality of leaflets configured to regulate flow through the prosthetic valve, and at least one support arm. The frame comprises a plurality of vertical posts that comprise a plurality of support posts, and a plurality of angled struts extending circumferentially between and interconnected with the vertical posts. Each support post extends between a post inflow end and an opposite post outflow end. The support posts comprise a plurality of commissure support posts and a plurality of non-commissural support posts. Each commissure support post comprises a commissure window. Each non-commissural support post is devoid of a commissure window. The at least one support arm extends from at least one of the vertical posts, and terminates at a free-ended tip which is biased radially away from the corresponding vertical post, defining a radial gap between the tip and the vertical post.

In some examples, the docking station comprises a radially collapsible and expandable docking frame comprising an inflow end portion and an outflow end portion, a sealing member disposed on the outflow end portion, and a valve seat extending from the outflow end portion and defining a diameter which is less than the diameter of a main body of the docking frame. The scaling member is configured to form a seal between the docking station and a body lumen, and defines a sealing member inflow end and a sealing member outflow end. The scaling member is retained between the annular frame of the prosthetic valve, in the radially expanded state, and the at least one support arm, such that the annular frame is positioned radially inward to the sealing member, and the tip is positioned radially outward to the sealing member.

According to some aspects of the disclosure, there is provided a method comprising: advancing a prosthetic valve in a radially compressed state toward a native heart valve; partially expanding the prosthetic valve; angularly orienting the prosthetic valve; and further expanding the prosthetic valve within a native annulus of the native heart valve. The prosthetic valve comprises an annular frame movable between a radially compressed state and a radially expanded state, a valvular structure mounted within the frame and comprising a plurality of leaflets configured to regulate flow through the prosthetic valve, and at least one support arm. The frame comprises a plurality of vertical posts that comprise a plurality of support posts, and a plurality of angled struts extending circumferentially between and interconnected with the vertical posts. Each support post extends between a post inflow end and an opposite post outflow end. The support posts comprise a plurality of commissure support posts and a plurality of non-commissural support posts. Each commissure support post comprises a commissure window. Each non-commissural support post is devoid of a commissure window. The at least one support arm extends from at least one of the vertical posts, and terminates at a free-ended tip which is biased radially away from the corresponding vertical post, defining a radial gap between the tip and the vertical post. Partially expanding the prosthetic valve is performed at a position in which the outflow end of the prosthetic valve is proximal to native leaflets of the native heart valve. Angularly orienting the prosthetic valve is performed such that commissures of the prosthetic valve are aligned with native commissures of the native leaflets.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, which proceeds with reference to the accompanying figures.

### BRIEF DESCRIPTION OF THE FIGURES

Some examples of the disclosed technology are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the disclosed technology. For the sake of clarity, some objects depicted in the figures are not to scale.

In the Figures:

FIG. 1 shows a sectional view of a healthy human heart.

FIG. 2A is perspective view of an exemplary mechanically expendable prosthetic valve that include support posts extending from support posts thereof.

FIG. 2B is a perspective view of the prosthetic valve of FIG. 2A with the skirt removed for clarity.

FIG. 2C a perspective view of the frame the prosthetic valve of FIGS. 2A-2B.

FIG. 2D is a top view of the frame of FIG. 2C.

FIG. 3 is an enlarged view in perspective of a portion of the frame with an exemplary support arm extending therefrom.

FIG. 4 shows an exemplary delivery assembly comprising a delivery apparatus carrying the prosthetic valve.

FIG. 5 shows one exemplary configuration of the prosthetic valve of FIGS. 2A-2D retained in a crimped state within a capsule.

FIG. 6 shows another exemplary configuration of the prosthetic valve of FIGS. 2A-2D retained in a crimped state within a capsule.

FIGS. 7-8 show perspective views of exemplary prosthetic valves that includes various shapes of support arms.

FIGS. 9A-9B show stages of an exemplary method for implanting the prosthetic valve of FIGS. 2A-2D.

FIG. 10 is perspective view of an exemplary prosthetic valve that includes three outflow support arms and three inflow support arms.

FIG. 11 is perspective view of an exemplary prosthetic valve that includes six outflow support arms and six inflow support arms.

FIG. 12 shows the prosthetic valve of FIG. 10 or 11 implanted within a native mitral valve.

FIG. 13 is a perspective view of an exemplary prosthetic valve that includes a sealing ring.

FIG. 14 shows the prosthetic valve of FIG. 13 implanted within a native mitral valve.

FIG. 15 is a sectional view of the human heart with an exemplary implementation of a docking station positioned in the inferior vena cava.

FIG. 16 is a side elevation view of a frame of an exemplary docking station.

FIG. 17 is a partial view in perspective of an exemplary docking station with a sealing member.

FIG. 18 is a perspective view of another exemplary prosthetic valve provided with outflow and inflow support arms.

FIG. 19 shows a partial view of a prosthetic assembly that include the prosthetic valve of FIG. 18 mounted in the docking station of FIG. 17.

FIGS. 20A-20B are perspective view of another example of a mechanically expendable prosthetic valve provided with support arms extending from post windows formed in support posts thereof, shown with and without a valvular structure mounted in the frame, respectively.

FIG. 21 shows a variety of types and positions of support arms extending from vertical posts of the frame.

FIG. 22 shows one exemplary configuration of the prosthetic valve of FIGS. 20A-20B retained in a crimped state within a capsule.

FIGS. 23A-23B show stages of an exemplary method for implanting the prosthetic valve of FIGS. 20A-20B.

FIG. 24 shows another exemplary prosthetic valve with distally-oriented support arms extending from post opening formed in inflow portions of the vertical posts.

FIGS. 25A-25B show stages of an exemplary method for implanting the prosthetic valve of FIG. 24.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (e.g., the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

It should be understood that the disclosed examples can be adapted to deliver and implant prosthetic devices in any of the native annuluses of the heart (e.g., the aortic, pulmonary, mitral, and tricuspid annuluses), and can be used with any of various delivery approaches (e.g., retrograde, antegrade, transseptal, transventricular, transatrial, etc.).

In order to avoid undue clutter from having too many reference numbers and lead lines on a particular drawing, some components will be introduced via one or more drawings and not explicitly identified in every subsequent drawing that contains that component.

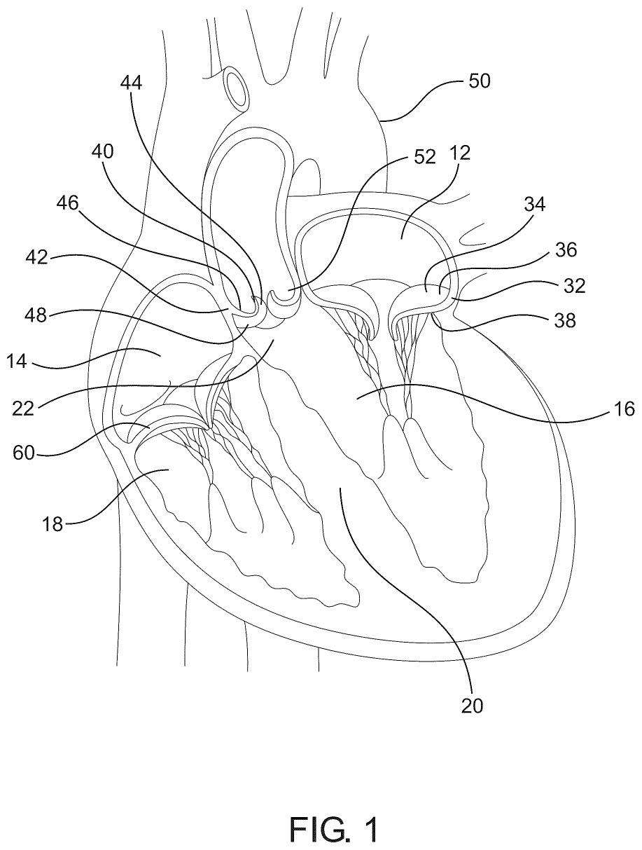

FIG. 1 shows a sectional view of a healthy human heart. The heart has a four-chambered conical structure that includes the left atrium 12, the right atrium 14, the left ventricle 16 and the right ventricle 18. The wall separating between the left and right sides of the heart is referred to as the septum 20. The native mitral valve 30 is positioned between the left atrium 12 and the left ventricle 16. The native tricuspid valve 60 is positioned between the right atrium 14 and the right ventricle 18. The native aortic valve 40 is positioned between the left ventricle 16 and the aorta 50. The initial portion of the aorta 50 extending from the native aortic valve 40 is the aortic root 52, and the adjoining part of the left ventricle 16 is the left ventricular outflow tract (LVOT) 22.

During the diastolic phase, or diastole, deoxygenated blood flows from the right atrium 14 into the right ventricle 18 through the tricuspid valve 60. During systole, leaflets of a normally functioning tricuspid valve 60 close to prevent the venous blood from regurgitating back into the right atrium 14. When the tricuspid valve 60 does not operate normally, blood can backflow or regurgitate into the right atrium 14.

The native mitral valve 30 comprises a mitral annulus 32 and a pair of mitral leaflets 34 extending downward from the annulus 32. When operating properly, the leaflets 34 function together to allow blood flow only from the left atrium 12 to the left ventricle 16. Specifically, during diastole, when the muscles of the left atrium 12 and the left ventricle 16 dilate, oxygenated blood flows from the left atrium 12, through the mitral valve 30, into the left ventricle 16. During systole, when the muscles of the left atrium 12 relax and the left ventricle 16 contacts, the blood pressure within the left ventricle 16 increases so as to urge to two mitral leaflets 34 to coapt, thereby preventing blood flow from the left ventricle 16 back to the left atrium 12.

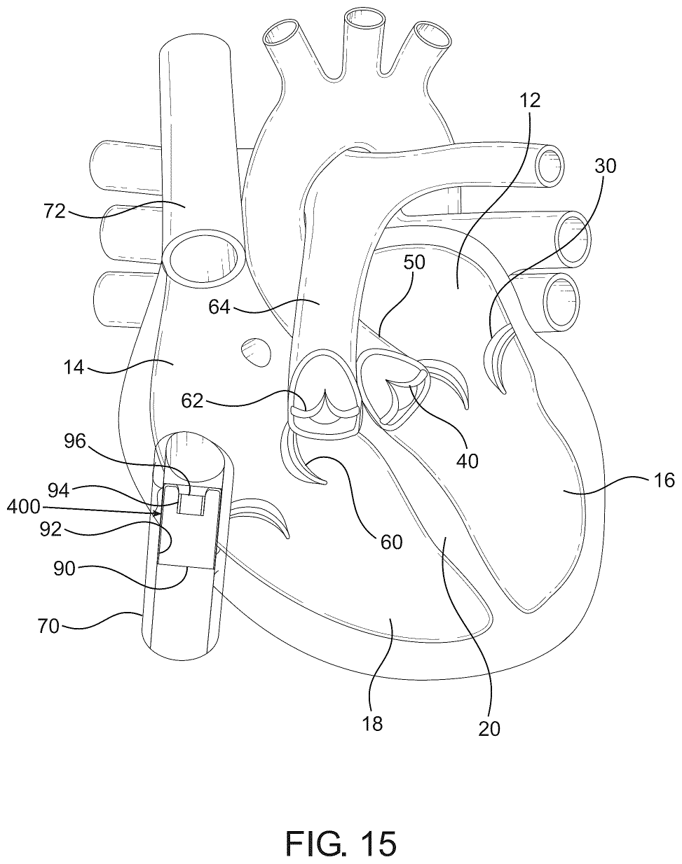

The native aortic valve 40 comprises an aortic annulus 42 and three aortic leaflets 44 extending upward (toward the aortic root 52) from the annulus 42. During systole, blood is expelled from the left ventricle 16, through the aortic valve 40, into the aorta 80. In addition, as illustrated for example in FIG. 15, the native pulmonary valve 62 separates the right ventricle 18 from the pulmonary artery 64. During systole, the right ventricle 18 contracts to force the blood collected in the right ventricle 18 through the pulmonary valve 62 and pulmonary artery 64 into the lungs. When either the native tricuspid valve 60, native mitral valve 30, native pulmonary valve 62, or native aortic valve 40, fails to function properly, a prosthetic replacement valve 100 can help restore functionality.

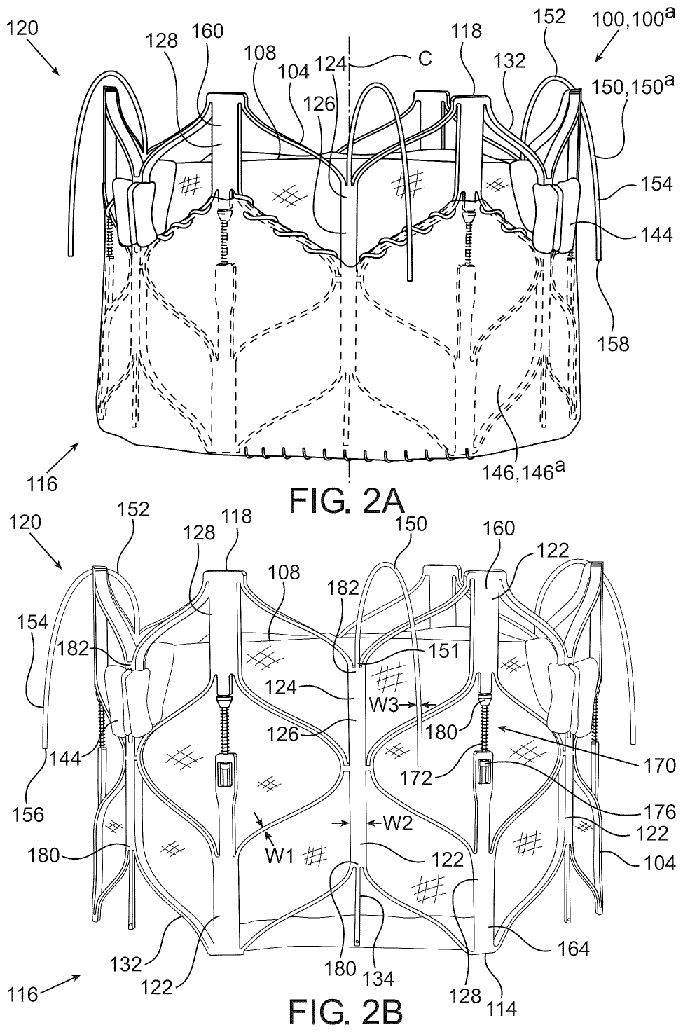

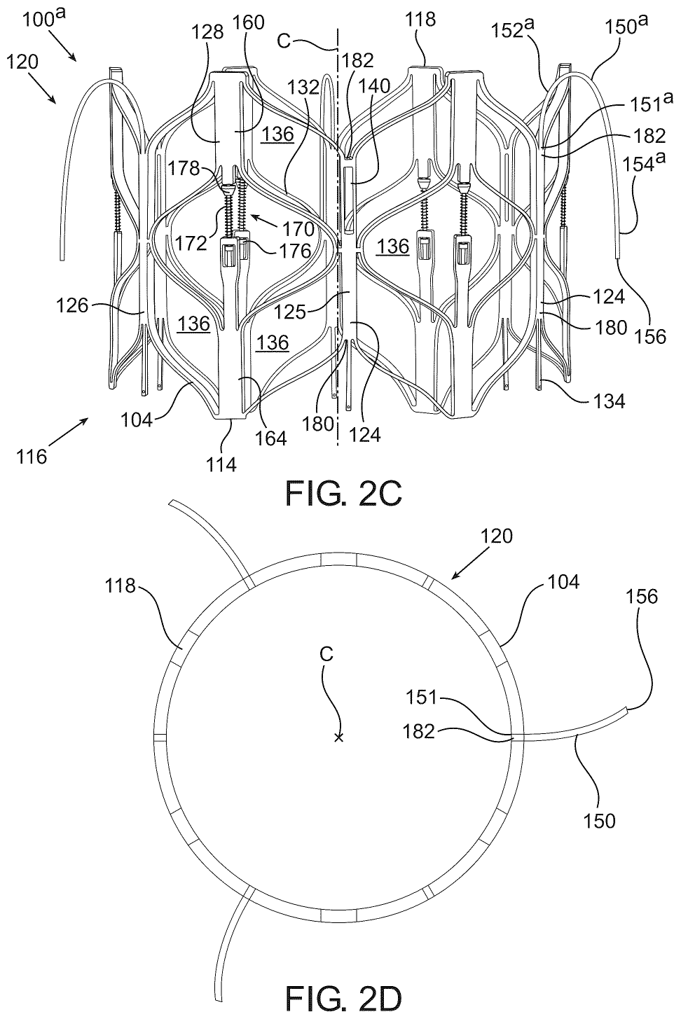

FIGS. 2A-2D illustrate a prosthetic valve 100, according to one example. The prosthetic valve 100 can be configured to replace a native heart valve (e.g., aortic, mitral, pulmonary, and/or tricuspid valves). The prosthetic valve 100 is illustrated as a mechanically expandable prosthetic valve that can be radially compressed for delivery to an implantation location within a patient's body and then radially expanded to a working diameter at the implantation location. The prosthetic valve 100 can include a frame 104 having an annular shape. The prosthetic valve 100 can further include a valvular structure 108 supported within and coupled to the frame 104. FIG. 2A shows a perspective view of a prosthetic valve 100 including a skirt 146 and a valvular structure 108 thereof. FIG. 2B shows the prosthetic valve 100 of FIG. 2A with the skirt 146 removed from view. FIG. 2B shows the frame 104 of the prosthetic valve 100 of FIGS. 2A-2B without any soft components, such as a skirt or a valvular structure. FIG. 2D shows a top view of the frame 104 of FIG. 2C.

In the example, the valvular structure 108 includes one or more leaflets 112 made of flexible material and configured to open and close to regulate blood flow. In one example, the valvular structure 108 can have three leaflets 112, which can be arranged to collapse in a tricuspid arrangement. The leaflets 112 can be made in whole or in part from pericardial tissue (e.g., bovine pericardial tissue), biocompatible synthetic materials, or various other suitable natural or synthetic materials.

As illustrated, the frame 104 has an inflow end 116, an outflow end 120, and a central longitudinal axis C extending in a direction from the inflow end 116 to the outflow end 120. The frame 104 can include a plurality of vertical posts 122 aligned with the central longitudinal axis C. The vertical posts 122 can include support posts 124 and actuation posts 128 spaced along a circumference of the frame 104. In one example, the support posts 124 and actuation posts 128 can be arranged in an alternating manner along the circumference of the frame 104. The frame 104 can further include a plurality of angled struts 132 extending circumferentially between adjacent support posts 124 and actuation posts 128 and interconnecting the support posts 124 and actuation posts 128. The angled struts 132, support posts 124, and actuation posts 128, define cells 136 of the frame 104. As illustrated, the angled struts 132 can have a curved shape.

One or more commissure windows 140 can be formed in one or more of the support posts 124. Commissures 144 can be formed at the commissure windows 140 to couple the leaflets 112 to the frame 104. Support posts 124 can include commissure support posts 125, which are support posts 124 that include commissure windows 140, and non-commissural support posts 126, which are support posts 124 devoid of commissure windows. The commissure support posts 125 and non-commissural support posts 126 can be arranged in an alternating manner along the circumference of the frame 104. In the illustrated example, frame 104 includes a total of six support posts 124, three of which are commissure support posts 125 and three of which are non-commissural support posts 126.

One or more of the support posts 124 can further include cantilevered struts 134 extending from the corresponding post inflow ends 180. In some examples, the cantilevered struts 134 can extend such that distal ends of the cantilevered struts 134 align with or substantially align with the inflow end 116 of the frame 104.

The prosthetic valve 100 can further include one or more skirts or sealing members. For example, the prosthetic valve 100 can include an inner skirt (not shown), mounted on the radially inner surface of the frame 104. The inner skirt can function as a scaling member to prevent or decrease perivalvular leakage, to anchor the leaflets 112 to the frame 104, and/or to protect the leaflets 112 against damage caused by contact with the frame 104 during crimping and during working cycles of the prosthetic valve 100. The prosthetic valve 100 can further include an outer skirt 146 mounted on the outer surface of the frame 104. The outer skirt 146 can function as a scaling member for the prosthetic valve 100 by sealing against the tissue of the native valve annulus and helping to reduce paravalvular leakage past the prosthetic valve 100. The inner and outer skirts can be formed from any of various suitable biocompatible materials, including any of various synthetic materials, including fabrics (e.g., polyethylene terephthalate fabric) or natural tissue (e.g., pericardial tissue). Further details regarding the use of skirts or sealing members in prosthetic valves can be found, for example, in U.S. Patent Application No. 62/854,702 and PCT Patent Application No. US2020/024559, each of which is incorporated by reference herein.

In some cases, as shown in the example illustrated in FIG. 2B, inflow edge portions of the leaflets 112 can be attached to the cantilevered struts 134 and/or to selected struts 132 of the frame 104. Alternatively or additionally, cantilevered struts 134 can prevent or mitigate portions of an outer skirt 146 from extending radially inwardly and thereby prevent or mitigate any obstruction of flow through the inflow end 116 of the frame 104 caused by the outer skirt 146. The cantilevered struts 134 can further serve as supports to which portions of the inner and/or outer skirts can be coupled, as shown in FIG. 2A. For example, sutures used to connect the inner and/or outer skirts can be wrapped around the cantilevered struts 134 and/or can extend through apertures formed at end portions of the cantilevered struts 134.

In some implementations, leaflets 112 can be sutured directly to angled struts 132 of the frame 104. In other implementation, inflow edge portions of the leaflets can be sutured to an inner skirt generally along the scallop line. The inner skirt can in turn be sutured, via one or more sutures, for example, to adjacent angled struts 132 of the frame 104.

Each support post 124, including any commissure support posts 125 or non-commissural support post 126, extends between a post inflow end 180 which is closer to the inflow end 116 of the valve 100, and a post outflow end 182 which is closer to the outflow end 120 of the valve 100. Two angled struts 132 intersect with each support post 124 at a post inflow end 180, which is circumferentially disposed between two adjacent inflow apices 114, such that a corresponding cantilevered strut 134 can extend distally from the post inflow end 180. Similarly, two angled struts 132 intersect with each support post 124 at a post outflow end 182, which is circumferentially disposed between two adjacent outflow apices 118.

As further shown, each commissure support post 125 and each non-commissural support post 126 also intersects, at a middle portion thereof, with four additional angled struts 132 extending from adjacent upper post members 160 and lower post members 164 on both sides, resulting in each support posts 124, and specifically, each commissure support post 125 and each non-commissural support post 126, intersecting with a total of at least eight curved struts extending from adjacent actuation posts 128.

In one example, the frame 104 can be adjusted between a radially expanded configuration and a radially compressed configuration by deflecting the angled struts 132. In one example, the frame 104 (e.g., the posts and struts) can be made of biocompatible plastically-expandable materials that will allow the frame 104 to be adjusted between the radially expanded configuration and radially compressed configuration. Suitable examples of plastically-expandable materials that can be used in forming the frame 104 include, but are not limited to, stainless steel, cobalt chromium alloy, and/or nickel titanium alloy (which can also be referred to as “NiTi” or “nitinol”).

In some examples, one or more actuators 170 can be coupled to the actuation posts 128, and used to adjust the frame 104 between the radially expanded configuration and the radially compressed configuration. In one example, each actuation post 128 can include an upper post member 160 and a lower post member 164 (the terms “upper” and “lower” are relative to the orientation of the prosthetic valve 100 in FIGS. 2A-2C) aligned with the longitudinal axis C and having opposing ends separated by a gap. The respective actuator 170 can be coupled to the post members 160, 164 and operable to increase or decrease the gap therebetween in order to radially compress or expand the frame 104. Angled struts 132 can converge with upper post members 160 to define outflow apices 118 at the outflow end 120. Angled struts 132 can similarly converge with lower post members 164 to define inflow apices 114 at the inflow end 116.

In one example, the actuator 170 can include an actuator rod 172 with an attached actuator head. In the example illustrated in FIGS. 2A-2C, the actuator rod 172 extends through or into the post members 160, 164 and across the gap therebetween. In the example illustrated in FIGS. 2A-2C, the actuator rod 172 is inserted into the upper post member 160 from the outflow end 120, and the actuator head (hidden from view in FIGS. 2A-2C) can be disposed or retained at the outflow apex 118 of the upper post member 160.

In some examples, the actuator rod 172 is externally threaded. As illustrated in FIGS. 2A-2C, the lower post member 164 can include a nut 176 with an internal thread to threadedly engage the actuator rod 172. In this case, the actuator rod 172 can be axially translated by rotating the actuator rod 172 relative to the nut 176. In some examples, the actuator rod 172 can be freely slidable relative to the upper post member 160. In other examples, the actuator rod 172 can threadedly engage the upper post member 160. The term “axially translated”, as used herein, refers to translation along an axis coinciding with or parallel to the central longitudinal axis C.

In one scenario, the actuator rod 172 can be rotated in a first direction to move the upper post member 160 towards the lower post member 164 and thereby decrease the size of the gap therebetween, which can have the effect of radially expanding the frame 104. In another scenario, the lower post member 164 may be held steady while the actuator rod 172 is rotated in a second direction to move the upper post member 160 away from the lower post member 164 and thereby increase the size of the gap therebetween, which can have the effect of radially compressing the frame 104.

The actuator rod 172 also can include a stopper 178 (e.g., in the form of a nut, washer or flange) disposed thereon. The stopper 178 can be disposed on actuator rod 172 such that it sits within the gap therebetween. Further, the stopper 178 can be integrally formed on or fixedly coupled to the actuator rod 172 such that it does not move relative to the actuator rod 172. Thus, the stopper 178 can remain in a fixed axial position on the actuator rod 172 such that it moves in lockstep with the actuator rod 172.

When the actuator rod 172 is rotated in a direction configured to collapse the prosthetic valve, the stopper 178 moves toward the outflow end 120 of the frame until the stopper 178 abuts the inflow end of the upper post member 160. Upon further rotation of the actuator rod 172, the stopper 178 can apply a proximally directed force to the upper post member 160 to radially compress the frame 104. Specifically, during crimping/radial compression of the prosthetic valve 100, the actuator rod 172 can be rotated in a direction that causes the stopper 178 to push against (i.e., provide a proximally directed force to) the inflow end of the upper post member 160, thereby causing the upper post member 160 to move away from the lower post member 164, and thereby axially elongating and radially compressing the prosthetic valve 100.

In an alternative implementation, some of the actuator rods 172 can be rotated in one direction while the other actuator rods 172 are rotated in an opposite direction simultaneously to either radially expand the frame or radially compress the frame. This counter-rotation of the actuator rods can be used to help reduce the likelihood of the entire frame 104 rotating about the central longitudinal axis C during rotation of the actuator rods 172 about their respective axes (e.g., when radially expanding the frame 104).

Additional examples of mechanically expandable valves can be found in International Application No. PCT/US2021/052745 and U.S. Provisional Applications Nos. 63/209,904 and 63/282,463, which are incorporated by reference herein.

A prosthetic valve 100 can further include support arms 150 extending from a support post 124. In the examples illustrated in FIGS. 2A-2C, three support arms 150 are shown to extend from non-commissural support posts 126, and more specifically, from post outflow ends 182 of the non-commissural support posts 126. While three support arms 150 are shown in the illustrated example, it is to be understood that any other number of support arms is contemplated, such as a single support arms, two support arms, or more than three support arms.

As shown, the actuation posts 128 are arranged in pairs, each pair including an upper post member 160 and a lower post member 164 which can be axially aligned with each other, and each pair of actuation posts 128 can be connected, such as via angled struts 132, to a commissure support post 125 on one side thereof, and to a non-commissural support post 126 on the other side. As shown, support posts 124 disposed between angled struts 132 can have a width, in the circumferential direction, that is greater than the width of other curved or angled struts 132 of the frame 104, so as to improve structural stability to the frame 104. This unique feature of increased width can be taken advantage of, to offer better support to the support arms 150 extending from ends thereof.

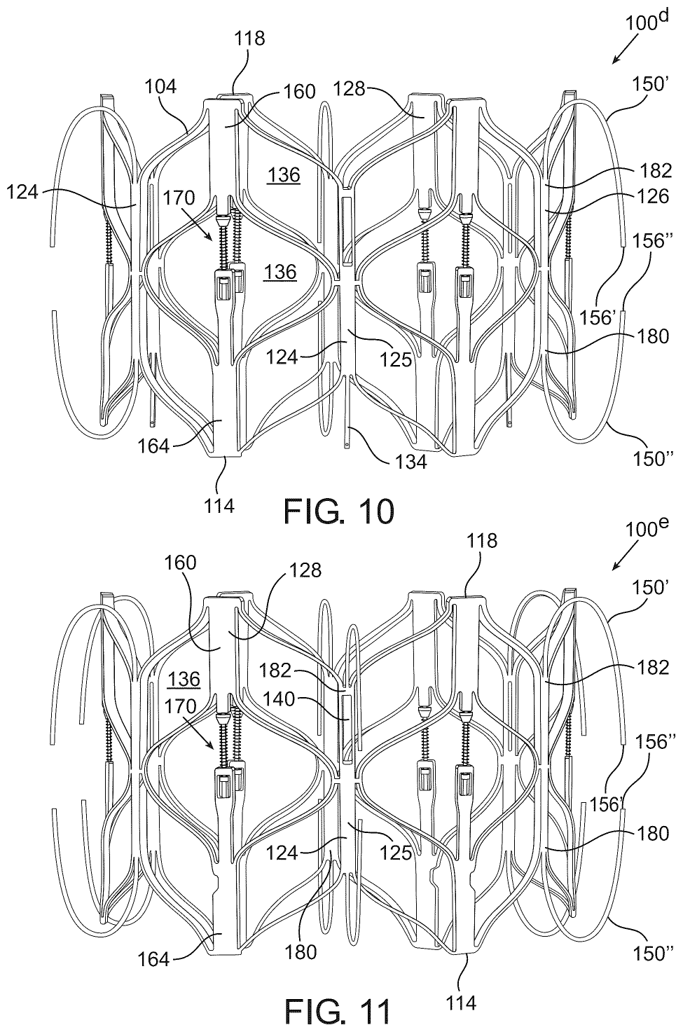

Each support arm 150 can extend from a base 151 at which it is attached to the support post 124, such as to a post outflow end 182 as illustrated in FIGS. 2A-2C, or a post inflow end 180 (as shown, for example, in FIG. 10), to a tip 156 which is biased radially away from the frame 104, and more specifically, from the corresponding support post 124, such that the tips 156 of corresponding support arms 150 are spaced away from the frame 104 in a free state of the support arms 150, referring to a state in which the support arms 150 are free to assume a pre-shaped configuration in the absence of external forces acting there-against. The support arms 150 can be formed from any suitable shape-memory material (e.g., Nitinol) such that the support arms resiliently extend radially away from the frame 104 when they are not constrained by an outer shaft 208 or a capsule 210, as will be further explained below.

In some examples, the support arm 150 can include a first arm portion 152 extending from the base 151, and a second arm portion 154 continuously extending from the first arm portion 152 and terminating at the tip 156. In a free state of the support arms 150, they extend generally radially outwardly from the frame 104 such that their tips 156 are generally spaced away from the rest of the frame 104. In one example, a support arm 150^{a} can have a C-shaped first arm portion 152^{a} (see FIG. 2C), while the second arm portion 154^{a} can be a linear or slightly curved portion of the arm (but with a curvature which is significantly less than the curvature of the first arm portion 152^{a}). The first arm portion 152^{a} can generally extend radially away from the corresponding support post 124, while the second arm portion 154^{a} can extend in the axial direction, toward the opposite end of the corresponding post 124. For example, for a support arm 150^{a} extending from the post outflow ends 182, the second arm portion 154^{a} can extend toward the post inflow end 180.

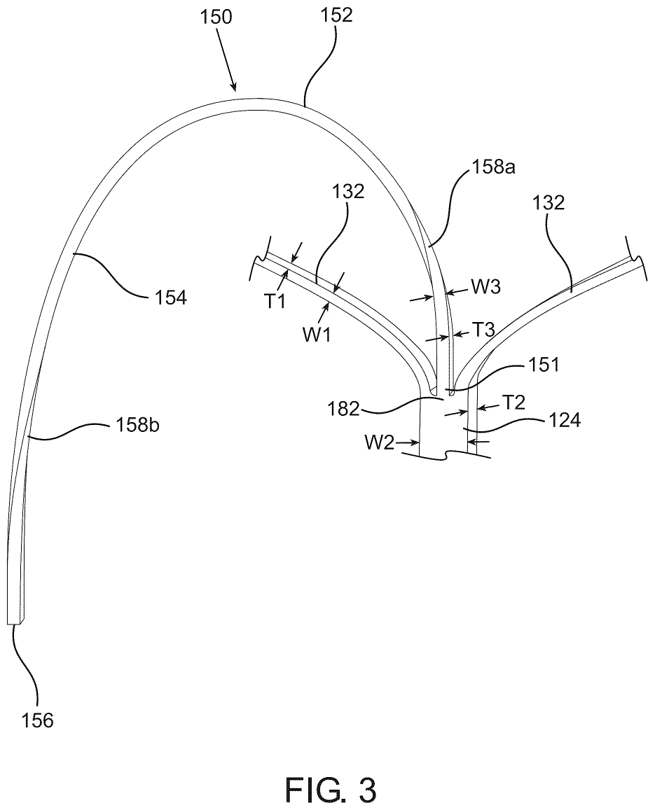

In some examples, as shown in FIG. 2D, any support arm 150 can be further circumferentially angled, meaning that in addition to being pre-shaped to deflect radially outwardly such that the tip 156 is spaced radially away from the rest of the frame 104, it can also deflect circumferentially such that the tip 156 is circumferentially offset from the corresponding base 151 and/or the corresponding support post 124. In some examples, as shown in FIG. 3, any support arm 150 can be further twisted. The support arms 150 can be twisted in a wide variety of different ways and can be twisted along their full length or just a portion of their length. The twists 158 aid in crimping or compressing of the prosthetic valve 100. In the illustrated example, a first twist 158a is included at or near (e.g., adjacent) the base 151 and a second twist 158b is included at or near (e.g., adjacent) the tip 156. While two twists 158 are shown in the example illustrated in FIG. 3, it is to be understood that any other number is contemplated, including a single twist, or more than two twists. In the illustrated example, the twists 158 are ninety degree twists, forming one-hundred-eighty total degrees of twist along a support arm 150 that include two twists.

Each angled strut 132 can have a thickness T1 in the radial direction, and a width W1 in the lateral or circumferential direction. Each support post 124 can have a thickness T2 in the radial direction, and a width W2 in the lateral or circumferential direction. Each support arm 150 can have a thickness T3 and a width W3. Since the support arm 150 can twist along its length, it is to be understood that the thickness T3 is defined in the radial direction and the width W3 is defined in the lateral or circumferential direction at least at or adjacent to the base 151.

In some implementations, as illustrated, the width W2 of the support posts is greater than the width W1 of the angled struts. Specifically, the thickness T1 and width W1 of the angled struts 132 is selected to allow them to change their angular orientation (in the lateral or circumferential direction) to allow the frame 104 to move between compressed and expanded configurations, while the axially-oriented vertical support posts 124 remain in the same orientation, and are usually wider to provide adequate support to the angled struts 132. Moreover, since some of the support posts 124 are commissure support posts 125, their width W2 can be selected to accommodate commissure windows 140 therein. Thus, in such prosthetic valves 100, the width W2 of the support posts 124 is greater than the width W1 of the angled struts 132, while the thickness T2 can be similar or equal to the thickness T1, especially if the frame 104 is manufactured by cutting (e.g., laser cutting) a tubular member having a uniform thickness. In some examples, the width W2 is at least two times greater than the width W1. In some examples, the width W2 is at least three times greater than the width W1.

The width W3 of the support arms 150 can be, in some implementations, less than the width W2 of the support posts 124 they extend from, to allow the support arms 150 to properly deflect away from the rest of the frame 104. While the thickness T3 can be similar to that of the thickness T2 and/or the thickness T1, especially if the support arms 150 are also cut (e.g., by laser cutting) from the same tube member as the rest of the frame 104, in other implementations, the thickness T3 can be less than the thickness T2. After being cut (e.g., laser cut from a tubular member), the support arms 150 can be heat-formed to a predefined shape, such as the shape of support arms 150^{a} described above, or other shapes as will be further described below. In some examples, the width W3 can be 70% or less than the width W2. In some examples, the width W3 can be 60% or less than the width W2. In some examples, the width W3 can be 50% or less than the width W2. In some examples, the width W3 can be 40% or less than the width W2. In some examples, the width W3 can be 30% or less than the width W2. In such implementations, the greater width W2 of the support arms 150 can provided improved structural to support arms 150.

In some examples, the support arms 150 can have a rectangular cross-section, such that the width W3 is greater than the thickness T3. In some examples, the thickness T3 can be 90% or less of the width W3, the thickness T3 can be 80% or less of the width W3, thickness T3 can be 70% or less of the width W3, thickness T3 can be 60% or less of the width W3, thickness T3 can be half or less of the width W3, thickness T3 can be 40% or less of the width W3, thickness T3 can be 30% or less of the width W3, thickness T3 can be ¼ or less of the width W3, or the thickness T3 can be 20% or less of the width W3.

The support arms 150 are configured to extend radially outwardly upon deployment of the prosthetic valve 100, and contact, abut or rest on an upper and/or lower abutment surfaces of the native annulus. For example, for a prosthetic valve 100 configured to be implanted within a native aortic valve 40, the tips 156 of support arms 150 extending from post outflow ends 182 can rest on aortic root abutment surface 46, which is the surface of the aortic annulus 42 facing the aortic root 52. Likewise, the tips 156 of support arms 150 extending from post inflow ends 180 can contact the LVOT abutment surface 48, which is the surface of the aortic annulus 42 facing the LVOT 22. Similarly, for a prosthetic valve 100 configured to be implanted within a native mitral valve 30, the tips 156 of support arms 150 extending from post outflow ends 182 can rest on mitral ventricular abutment surface 38, which is the surface of the mitral annulus 32 or native mitral leaflets 34 facing the left ventricle 16. Likewise, the tips 156 of support arms 150 extending from post inflow ends 180 can contact the mitral atrial abutment surface 36, which is the surface of the mitral annulus 32 or native mitral leaflets 34 facing the left atrium 12. Thus, the tips 156 can be atraumatic to avoid damaging the abutment surfaces they are configured to contact or rest on. For example, the tips 156 may have smooth contact surfaces that may be flattened or curved and are not configured to penetrate the tissue they contact and/or rest on. The tips 156 can be covered in some implementations. In some examples, the tips 156 can be configured to be flexible to allow for reduction of possible trauma to the tissue upon contact.

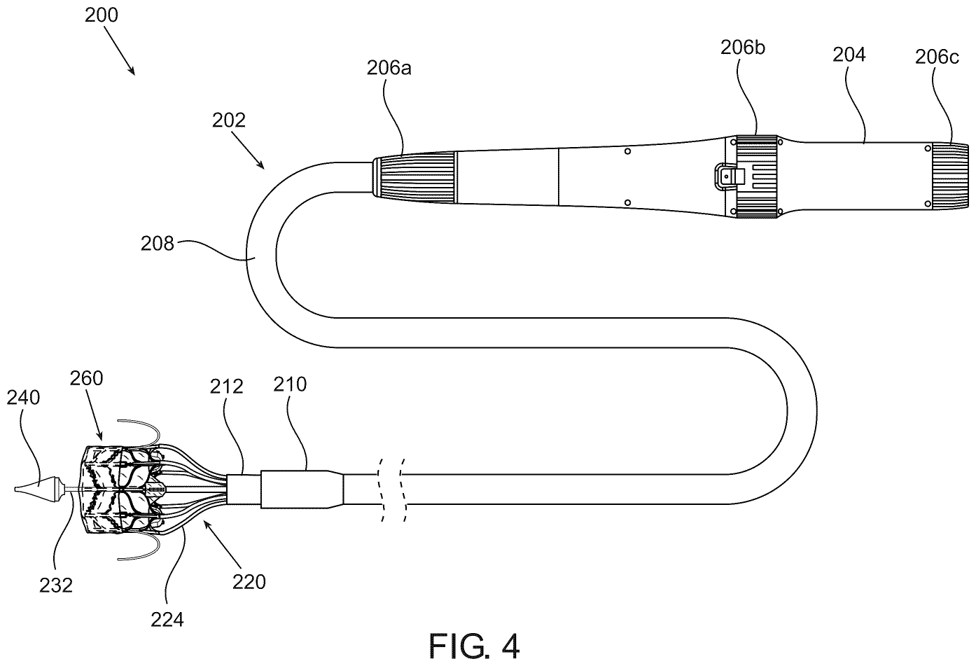

FIG. 4 illustrates a delivery assembly 200, which can include a prosthetic valve 260 and a delivery apparatus 202, according to some examples. The delivery apparatus 2—can be used to deliver a mechanically expandable prosthetic valve 260 described herein (e.g., prosthetic valve 100 or 500). The prosthetic valve 260 can be releasably coupled to the delivery apparatus 202. It should be understood that the delivery apparatus 202 can be used to implant prosthetic devices other than prosthetic valves, such as stents or grafts.

The delivery apparatus 202 in the illustrated example generally includes a handle 204, an outer elongated shaft 208 extending distally from the handle 204 and at least one actuator assembly 220 extending distally through the outer shaft 208. The delivery apparatus 202 can also include an elongated nosecone shaft 232 extending distally from the handle 204 through the outer shaft 208. A nosecone 240 can be connected to the distal end of the nosecone shaft 232. The at least one actuator assembly 220 can be configured to radially expand and/or radially collapse the prosthetic valve 260 when actuated.

As illustrated, one actuator assembly 220 can be provided for each actuator (e.g., actuator 170 or 570) on the prosthetic valve 260. For example, six actuator assemblies 220 can be provided for a prosthetic valve 260 having six actuators. In other configurations, however, any greater or fewer number of actuator assemblies can be present.

The distal end portion of the shaft 208 can be sized and shaped to house the prosthetic valve 260 in a radially compressed, delivery state during delivery of the prosthetic valve through, for example, the vasculature of a patient. In this way, the distal end portion of shaft 208 functions as a delivery sheath or capsule for the prosthetic valve during delivery.

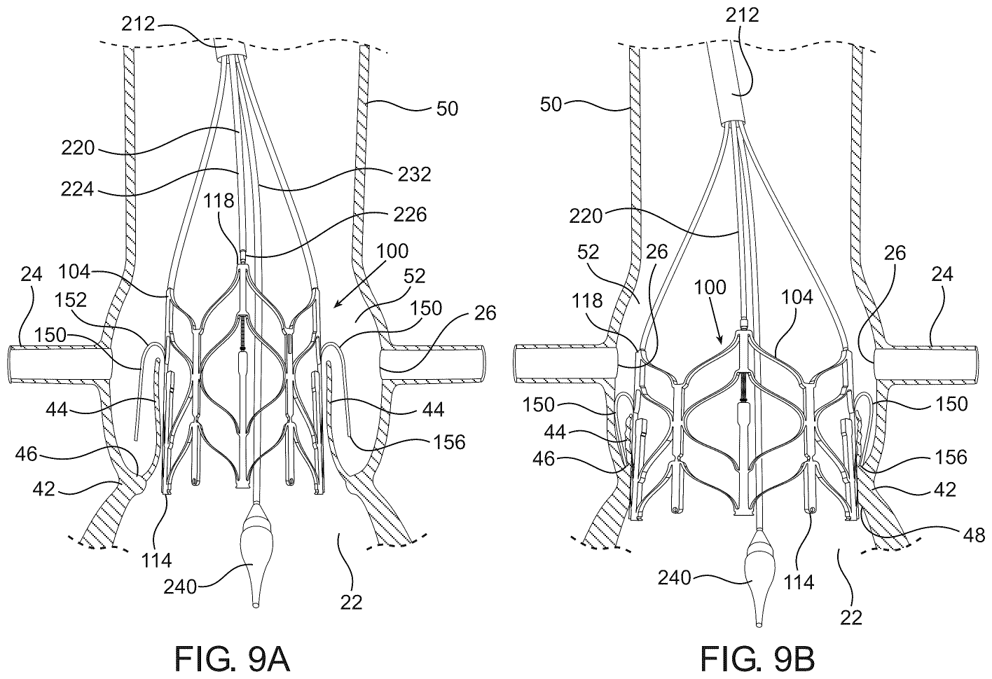

The actuator assemblies 220 can be releasably coupled to the prosthetic valve 260. For example, in the illustrated configuration, each actuator assembly 220 can be coupled to a respective actuator of the prosthetic valve 260. Each actuator assembly 220 can comprise a support tube 224, an actuator member 226 (hidden within support tube 224 in FIG. 4, exposed in FIG. 9A), and optionally a locking tool. When actuated, the actuator assembly can transmit pushing and/or pulling forces to portions of the prosthetic valve to radially expand and collapse the prosthetic valve as previously described. The actuator assemblies 220 can be at least partially disposed radially within, and extend axially through, one or more lumens of the outer shaft 208. For instance, the actuator assemblies 220 can extend through a central lumen of the shaft 208 or through separate respective lumens formed in the shaft 208.

The terms “releasably coupled” or “releasably attached”, as used herein, are interchangeable, and refer to two components coupled in such a way that they are coupled together and can be separated without plastically deforming either of the components.

Although not illustrated, the delivery apparatus 202 can include, in some implementations, a multi-lumen delivery shaft 212 extending through the lumen of the outer shaft and having a plurality of lumens therein. Any of the nosecone shaft 232 and/or actuation assemblies 220 can extend through lumens of the multi-lumen delivery shaft 212.

The actuator member 226 of each actuator assembly 220 can be releasably coupled to a respective actuator of the prosthetic valve (e.g., actuator 170). The support tube 224 of each actuator assembly 220 can abut an adjacent portion of the frame of the prosthetic valve, such as an outflow apex (e.g., apex 118). In this manner, during valve expansion, the support tubes 224 can prevent movement of the outflow end of the prosthetic valve relative to the delivery apparatus while the actuator members of the actuator assemblies 220 can actuate the actuators of the prosthetic valve and cause the inflow end of the prosthetic valve to move toward the outflow end of the prosthetic valve.

The handle 204 of the delivery apparatus 202 can include one or more control mechanisms (e.g., knobs 206 or other actuating mechanisms) for controlling different components of the delivery apparatus 202 in order to expand and/or deploy the prosthetic valve 260. For instance, in the illustrated example, the handle 204 comprises first, second, and third knobs 206a, 206b, and 206c.

The first knob 206a can be a rotatable knob configured to produce axial movement of the outer shaft 208 relative to the prosthetic valve 260 in the distal and/or proximal directions in order deploy the prosthetic valve from the delivery sheath once the prosthetic valve has been advanced to a location at or adjacent the desired site of implantation within a patient. For instance, rotation of the first knob 206a in a first direction (e.g., clockwise) can retract the outer shaft 208 proximally relative to the prosthetic valve 260 and rotation of the first knob 206a in a second direction (e.g., counterclockwise) can advance the outer shaft 208 distally. In other configurations, the first knob 206a can actuated by sliding or moving the knob 206a axially, such as puling and/or pushing the knob. In still further configurations, actuations of the first knob 206a, such as by rotation or sliding the first knob 206a, can produce axial movement of the actuator assemblies 220 and thereby the prosthetic valve 260 relative to the delivery sheath to advance the prosthetic valve distally from the sheath.

In one example, a capsule 210 can be attached to a distal end of the outer shaft 208. Axial movement of the outer shaft 208 in a distal direction relative to the other shafts and prosthetic valve can move the capsule 210 over the distal end portions of the actuation assemblies 220 and the prosthetic valve 260 (i.e., when the prosthetic valve 260, such as prosthetic valve 100 or 500, is in the radially compressed configuration) such that the prosthetic valve 260 is enclosed within the capsule 210. Axial movement of the outer shaft 208 in a proximal direction relative to the other shafts and the prosthetic valve can retract the capsule 210 from the prosthetic valve 260, exposing the prosthetic valve, for example, for deployment at an implantation location.

The second knob 206b can be a rotatable knob configured to produce radial expansion and/or contraction of the prosthetic valve 260. For instance, rotation of the second knob 306b can move the actuator members and the support tubes 224 of actuator assemblies 220 axially relative to one another. The actuator members or drivers of actuator assemblies 220 in turn cause corresponding movement of the actuators (e.g., actuators 170 or 570) of the prosthetic valve. Rotation of the second knob 206b in a first direction (e.g., clockwise) can radially expand the prosthetic valve 260 and rotation of the second knob 206b in a second direction (e.g., counterclockwise) can radially collapse the prosthetic valve 260. In other configurations, the second knob 206b can be actuated by sliding or moving the knob 206b axially, such as pulling and/or pushing the knob.

The third knob 206c can be a rotatable knob configured to retain the prosthetic valve 260 in an expanded state. For instance, the third knob 206c can be operatively connected to a proximal end portion of the locking tool of each actuator assembly 220. Rotation of the third knob 206c in a first direction (e.g., clockwise) can rotate each locking tool to advance the locking nuts to their distal positions to resist radial compression of the frame of the prosthetic valve. Rotation of the knob 206c in the opposite direction (e.g., counterclockwise) can rotate each locking tool in the opposite direction to decouple each locking tool from the prosthetic valve 260. In other configurations, the third knob 206c can be actuated by sliding or moving the third knob 206c axially, such as pulling and/or pushing the knob. In some embodiments, the prosthetic valve can be self-locking, in which case a locking tool is not required. For example, the frame of the prosthetic valve can include locking features that automatically engage the actuator members of the prosthetic valve to resist radial compression of the prosthetic valve after it is expanded, such as disclosed in U.S. Application Nos. 63/085,947, 63/138,599, and 63/179,766.

Although not shown, the handle 204 can include a fourth rotatable knob operative connected to a proximal end portion of each actuator member. The fourth knob can be configured to rotate each actuator member, upon rotation of the knob, to unscrew each actuator member 226 from the proximal portion of a respective actuator. As described above, once the locking tools and the actuator members are uncoupled from the prosthetic valve 260, they can be removed from the patient.

In particular implementations, the delivery assembly 202 including the delivery apparatus 202 with the prosthetic valve 260 assembled thereon, can be packaged in a sterile package that can be supplied to end users for storage and eventual use. In some examples, the leaflets of the prosthetic valve (typically made from bovine pericardium tissue or other natural or synthetic tissues) are treated during the manufacturing process so that they are completely or substantially dehydrated and can be stored in a partially or fully crimped state without a hydrating fluid. In this manner, the package containing the prosthetic valve 260 and the delivery apparatus 202, respectively, can be free of any liquid. Methods for treating tissue leaflets for dry storage are disclosed in U.S. Pat. Nos. 8,007,992 and 8,357,387, both of which documents are incorporated herein by reference.

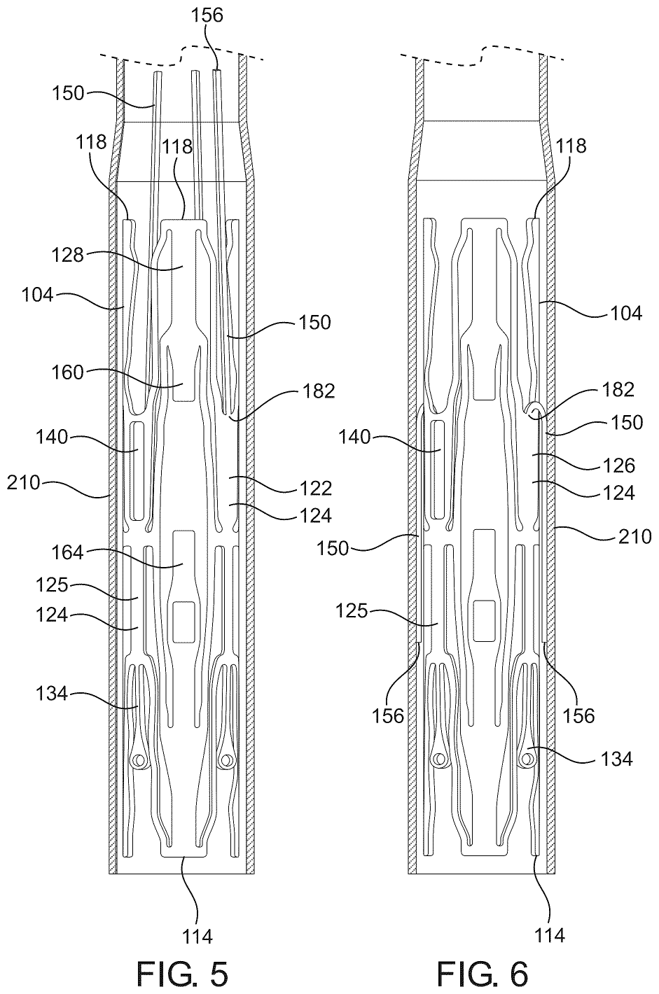

FIGS. 5-6 shows various configurations by which a prosthetic valve 100 with self-expandable support arms 150 can be retained in an undeployed state within a capsule 210 or a sheath of the delivery apparatus 202 during delivery to the site of implantation. For simplicity, soft components, such as a valvular structure 108 or a skirt 146, are not shown. While the prosthetic valve 100 is illustrated to be retained in a crimped state within a capsule 210 in FIGS. 5-6, it is to be understood that the same configurations apply to a delivery apparatus that does not necessarily include a capsule, in which case the valve 100 can be similarly retained within a distal portion of a shaft of the delivery apparatus, such as the outer shaft 208. The actuator assemblies 220, which can be coupled to the prosthetic valve 100 during delivery, as well as actuators 170 and soft components of the valve 100, are removed from view in FIGS. 5-6 for clarity.

FIG. 5 illustrates one exemplary configuration with “flipping” support arms. During delivery, the support arms 150 can be partially or completely straightened, as shown, in their undeployed state. The support arms 150 can be restrained from radially expanding outward by the constrictive force of the capsule 210. Depending on the length of the support arms 150, this configuration can result in the tips 156 disposed proximal to the outflow apices 118 of the frame 104 if for support arms 150 that extend from post outflow ends 182, or disposed distal to the inflow apices 114 of the frame 104 if for support arms 150 that extend from post inflow ends 180. When the support arms 150 are freed from the capsule 210 during deployment of the prosthetic valve 100, they spring back to their pre-formed shape, as shown in FIGS. 2A-2D.

FIG. 6 illustrates another exemplary configuration in which the support arms 150 are compressed, in their undeployed folded configuration, between the rest of the frame 104 and the inner walls of the capsule 210. The support arms 150 can be restrained from deployment by the capsule 210. Depending on the length of the support arms 150, this configuration can result in the tips 156 disposed between the inflow end 116 and the outflow end 120 of the valve. When the support arms 150 are freed from the capsule 210 during deployment of the prosthetic valve 100, they spring outwardly to their pre-formed shape, as shown in FIGS. 2A-2D.

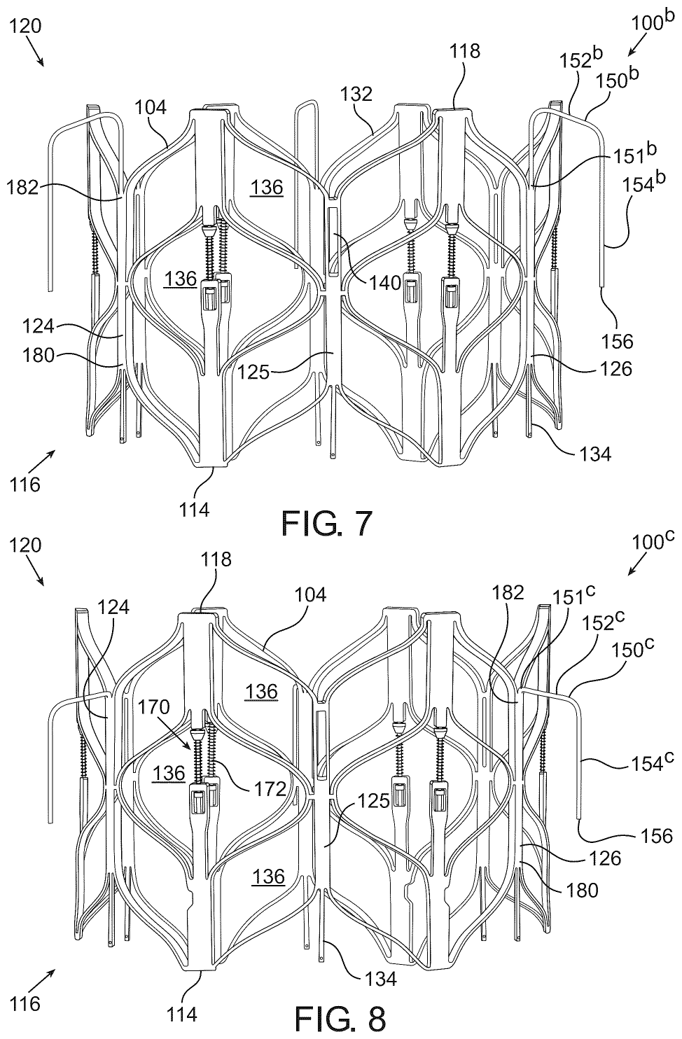

FIGS. 7-8 show additional optional shapes of the support arms 150 in their free, deployed state. FIG. 7 shows an exemplary support arm 150^{b} that includes a U-shaped or L-shaped first arm portion 152^{b}, and a relatively linear second arm portion 154^{b} extending axially therefrom. FIG. 8 shows an exemplary support arm 150^{c} that includes a relatively linear first arm portion 152^{c} extending radially away from the base 151^{c}, and a relatively linear second arm portion 154^{c} extending axially therefrom. It is to be understood that other pre-formed shapes are contemplated, configured to deflect the support arms 150 radially outwardly to the rest of the frame 104.

FIG. 9A-9B show stages of an exemplary method for implantation of a prosthetic valve 100 within a native aortic annulus 42. For simplicity, soft components, such as a valvular structure 108 or a skirt 146, are not shown. The prosthetic valve 100 can be coupled to a delivery apparatus 202, which can be used to deliver, position, and secure the prosthetic valve 100 in a native heart valve annulus. In the illustrated implantation procedure, the prosthetic valve 100 is implanted in a native aortic annulus 42 using a transfemoral delivery approach. In other examples, the prosthetic valve 100 can be implanted at other locations (e.g., a mitral valve, a tricuspid valve, and/or a pulmonary valve), within previously-implanted prosthetic valve, and/or using other delivery approaches (e.g., transapical, transaortic, transseptal, etc.).

The prosthetic valve 100 can be releasably coupled, as described above, to the actuator assemblies 220 of delivery apparatus 202, and advanced in a compressed state through the patient's vasculature toward the site of implantation (e.g., the aortic annulus). Upon reaching the site of implantation, the prosthetic valve 100 can be deployed by pushing it distally out of the capsule 210 and/or outer shaft 208, or by proximally pulling the capsule 210 and/or outer shaft 208 relative to the prosthetic valve 100, which allows the support arms 150 to spring out radially away from the rest of the frame 104 as described above with respect to FIGS. 5-6, for example. The actuation assemblies 220 can then be utilized, as described above, to radially expand the prosthetic valve 100, at least to a partially expanded diameter, as shown in FIG. 9A.

A significant advantage of mechanically expandable prosthetic valves is that the expansion mechanism allows them to be controllably and gradually expanded, as opposed to balloon expandable valves or self-expandable valves, which are conventionally expanded to their final functional diameter in a relatively rapid manner with limited control over the expansion process. This can be taken advantage of for properly positioning the support arms in a desired orientation, relative to the native anatomy. For example, as shown in FIG. 9A, actuator assemblies 220 can be utilized to partially expand the prosthetic valve 100 to a partially expanded diameter, which is greater than the crimped diameter but less than the final functional diameter, such that the frame 104, and optionally even the support arms 150, are not yet immovably pressed within the native annulus. This allows the prosthetic valve 100 to be angularly oriented to a desired angular orientation, for example with respect to the aortic leaflets 44.

As shown in FIG. 9A, when the prosthetic valve 100 is deployed within the native aortic valve 40, the support arms 150 can be positioned over and/or around the aortic leaflets 44. As shown in FIGS. 2A-2D, a prosthetic valve 100 can include a total of six support posts 124, three of which are commissure support posts 125 to which commissures 144 are coupled, and three of which are non-commissural support posts 126 disposed between the commissure support posts 125. In one configuration, the prosthetic valve 100 can include one or more (e.g., three as in the illustrated examples) support arms 150 extending from the post outflow ends 182 of the non-commissural support posts 126, while the commissure support posts 125 remain devoid of support arms. When implanted in a native aortic valve 40, it may be desired to position the support arms 150 over the native aortic leaflets 44 between the native commissures, such that the tips 156 can abut aortic root abutment surface 46 instead of contacting the native commissures.

The prosthetic valve 100 can be deployed out of the capsule at a position which is proximal to the native aortic leaflets 44, allowing the support arms 150 to spring radially outwards, after which the prosthetic valve 100 can be distally advanced to position the native aortic leaflets 44 between the frame 104 and the support arms 150. Partial expansion of the prosthetic valve 100 can be performed upon deployment, prior to axial advancement, and/or during and/or after axial advancement of the valve 100. In the partially expanded diameter of the prosthetic valve 100, shown in FIG. 9A, it can be angularly reoriented, if needed, to align the commissures 144 of the prosthetic valve 100 with the native commissures between the native aortic leaflets 44.

Further expansion of the prosthetic valve 100 to the functional size, which can be optionally accompanied by further axial displacement relative to the native aortic annulus 42, serves to anchor the prosthetic valve 100 in position, wherein the support arms 150 may press against the native annulus for migration resistance. For example, the tips 156 can rest over the aortic root abutment surface 46, so as to resist unintentional distally oriented migration of the prosthetic valve 100 toward the left ventricle 16.

In some cases, the support arms 150 can extend around native leaflets and potentially grasp the native leaflets, as shown in FIG. 9B. For example, when the prosthetic valve 100 is further expanded by the actuator assemblies 220 from the partially expanded diameter shown in FIG. 9A to the desired functional diameter shown in FIG. 9B, the support arms 150 can be pressed between the surrounding aortic wall and the expanded frame 104, grasping portions of the native aortic leaflets 44 between the support arms 150 and the frame 104. The frame 104 and at least some portion of the support arms 150, such as the tips 156, can approximate each other in such procedures, optionally pinching the native leaflets 44 therebetween. In some cases, the native leaflets 44 can be bunched up between the support arms 150 and the rest of the frame 104. This can provide for greater securement of the prosthetic valve 100 to the leaflets 44, which may be specifically advantageous in some pathologies, such as Aortic Insufficiency, in which the native leaflets 44 do not include sufficient internal calcifications to warrant proper retaining force of a prosthetic valve pressed there-against. In alternative implementations, the support arms 150 do not necessarily hook the native leaflets.

While optional angular orientation of the prosthetic valve 100 is described above, in a partially expanded state thereof, it is to be understood that in alternative implementations, active angular orientation may not be required, relying on the natural tendency of the support arms 150 to slip along the native leaflets 44 to a position between the native commissures, during continuous expansion of the valve 100. In such implementations, the mechanical expansion mechanism of a mechanically expandable prosthetic valve 100 is still advantageous in that it allows sequential controlled expansion of the valve 100 at a rate that will allow the support arms 150 to be self-oriented to the proper positions along the native aortic leaflets 44, whereas abrupt expansion of conventional self-expandable valves, for example, may result in a less desirable outcome with respect to the position of the support arms 150 relative to the native anatomy.

Utilization of support arms 150 engaged with portions of the native tissue, such as annular abutment surfaces and/or native leaflets, can advantageously prevent or reduce the prevalence of axial movement of the prosthetic valve 100 in a direction opposite to the side the arms 150 extend from, and can also help ensure that the prosthetic valve 100 is perpendicular to the annulus and prevents undesirable “rocking” or tilting of the frame. By engaging the native annulus, and optionally the native leaflets, the support arm can assist in more evenly distributing the load to achieve equilibrium, thus providing a more robust implantation.

In some implementations, the gap formed between the tips 156 and the frame 104 is configured to allow the support arms 150 to cover the native aortic leaflets 44 during gradual controlled expansion of the prosthetic valve 100. When further utilized to accommodate native aortic leaflets 44 therein, the native leaflets can be folded, for example by first arm portions 152 of the support arms 150, toward the native aortic annulus 42, distancing them away from the coronary arteries 24 to avoid obstruction of the ostia of the coronary arteries 24. Native leaflets bunched up within support arms 150 can also improve PVL sealing around the frame 104.

After reaching the final functional expanded diameter, the actuator assemblies 220 can be uncoupled from the actuators 170 and the delivery apparatus 202 can then be withdrawn from the patient's body, leaving the prosthetic valve 100 within the aortic annulus 42 to regulate blood flow from the left ventricle 16 into the aorta 50. While illustrated for use in a native aortic valve 40 in FIGS. 9A-9B, it is to be understood that a mechanically expandable prosthetic valve 100 with support arms 150 can be similarly implanted within a native tricuspid valve 60, a native mitral valve 30 valve, or any other orifice.

As mentioned, a prosthetic valve 100 can include any number of support arms 150. Thus, the plural use of the term “support arms 150” is not meant to be limiting, and may refer to implementation in which a single support arm 150 is included. For example, a prosthetic valve 100 configured for deployment within a native valve, such as the native aortic valve 40 described above with respect to FIGS. 9A-9B, can include any number of support arms, such as three support arms as shown in FIGS. 2A-2D, more or less than three arms, or even a single support arm 150 that can extend, for example, from a post outflow end 182 of one of the non-commissural support posts 126.

Support arms 150 can include outflow support arms 150′, extending from post outflow ends 182, as well as inflow support arms 150″, extending from post inflow ends 180. FIG. 10 shows another exemplary configuration of a mechanically expandable prosthetic valve 100^{d} that includes both outflow support arms 150′ and inflow support arms 150″. While three outflow support arms 150′ and three inflow support arms 150″ are illustrated, it is to be understood that any other number of any type of support post(s) is contemplated.

In some examples, a prosthetic valve 100 can include an equal number of outflow support arms 150′ and inflow support arms 150″, as illustrated in FIG. 10. In alternative configurations, the number of outflow support arms 150′ can be different from the number of inflow support arms 150″. In some examples, the prosthetic valve 100 can include one or more inflow support arms 150″ without any outflow support arms.

In some examples, the outflow support arms 150′ and the inflow support arms 150″ can be aligned, extending from opposite sides of the same support posts 124, as shown in FIG. 10. In other examples, the outflow support arms 150′ and inflow support arms 150″ can be arrange in any other non-aligned arrangement, including in a staggered arrangement relative to each other. While the support arms 150′ and 150″ are shown to extend from post outflow ends 182 and post inflow ends 180 of non-commissural support posts 126, it is to be understood that in alternative implementations, support arms 150 of any type can extend from post outflow ends 182 and/or post inflow ends 180 of commissure support posts 125.

When implanted within a native aortic valve 40 for example, as described above with respect to FIGS. 9A-9B, inflow support arms 150″ of the type shown for prosthetic valve 100^{d}, for example, can atraumatically engage with the LVOT abutment surface, to prevent undesirable axial movement of the prosthetic valve in the proximal direction, toward the aorta 50. Since it may be desired to avoid outflow support arms 150′ from being aligned with native commissures of an aortic valve 40, prosthetic valves 100 for such implantations can include outflow support arms 150′ that extend solely from non-commissural support posts 126, with no outflow arms extending from commissure support posts 125. However, since the ventricular side of the aortic valve 40 does not include native leaflets or commissures, such that prosthetic valve 100 for such implantations can include inflow support arms 150″ that extend from any support posts 124, including commissure support posts 125. Thus, prosthetic valve 100 can include a first number of outflow support arms 150′ (e.g., three), and a second number of inflow support arms 150″ (e.g., six).

FIG. 11 shows another exemplary configuration of a mechanically expandable prosthetic valve 100^{e} that includes outflow support arms 150′ and inflow support arms 150″ extending from all of the support posts 124, resulting, for example, in a total of six inflow support arms 150″ and six outflow support arms 150′, though any other number is contemplated.

When a mechanically expandable prosthetic valve 100 includes inflow support arms 150″, they can be formed to extend from the corresponding support posts 124 instead of cantilevered struts 134, and can be shaped and designed to support portions of inner and or outer skirts that can be coupled thereto, in a similar manner described above with respect to the cantilevers struts 134. For example, skirts and/or leaflets of the prosthetic valve 100 can be coupled (e.g., sutured) to first arm portions 152 of selected or all inflow support arms 150″. In some examples, as illustrated in FIG. 10 for prosthetic valve 100^{d}, both inflow support arms 150″ and cantilevered struts 134 can be included. Exemplary prosthetic valve 100^{d} is shown to include three inflow support arms 150″, and three cantilevers struts 134 intermittently disposed therebetween, together configured to support skirts and/or leaflets that can be coupled thereto. FIG. 11 illustrates another configuration of a prosthetic valve 100^{e} that includes inflow support arms 150″ without any cantilevered struts 134, wherein some or all of the inflow support arms 150″ can be configured to support skirts and/or leaflets that can be coupled thereto.

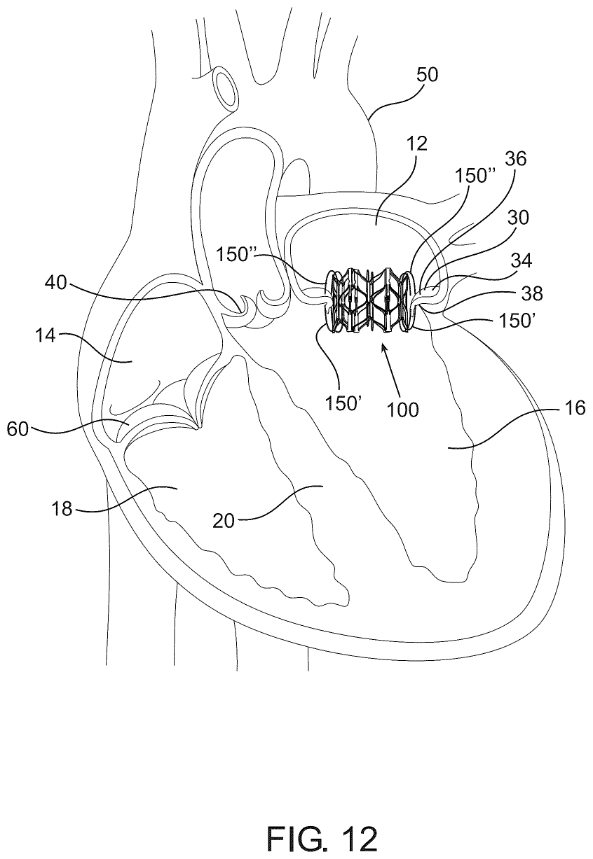

FIG. 12 shows a mechanically expandable prosthetic valve 100 that can be implanted within the mitral valve. The prosthetic valve illustrated in FIG. 12 is of the type of prosthetic valve 100^{e} illustrated in FIG. 11, including both outflow support arms 150′ and inflow support arms 150″, optionally extending from all support posts 124, though any other configuration is contemplated. A prosthetic valve 100 that includes both outflow and inflow support arms 150′, 150″ can be supported at opposing sides of the native valve, such as the mitral valve 30. The opposing support arms 150 are disposed on opposite sides of the mitral annulus 32, wherein the tips 156 of the outflow support arms 150′ can atraumatically engage with the mitral ventricular abutment surface 38, and the tips 156 of the inflow support arms 150″ can atraumatically engage with the mitral atrial abutment surface 36, as shown. This configuration allows the prosthetic valve 100 to be held securely in position without requiring a substantial radial force against the native annulus, resisting axial migration of the valve 100 in both directions, as the mitral annulus 32 is grasped between the outflow support arms 150′ and the inflow support arms 150″.

Conventional prosthetic valves may be appropriately sized for placement inside many native cardiac valves or orifices, such as within a native aortic annulus 40. However, with larger native valves (e.g., a native tricuspid valve 60 or a native mitral valve 30), a conventional prosthetic valve might be too small to secure into the larger annulus. In this case, the prosthetic valve may not be large enough to sufficiently expand inside and properly seal against the native the native annulus. Support arms 150 extending from opposing sides of the support posts 124 can securely grasp the native mitral annulus 32, for example, between their opposingly directed tips 156, such that the frame 104 need not rely on a pressure fit, or friction fit, between the outer surface of the valve 100 and the inner surface of the mitral annulus 32 for adequate prosthetic valve retention.

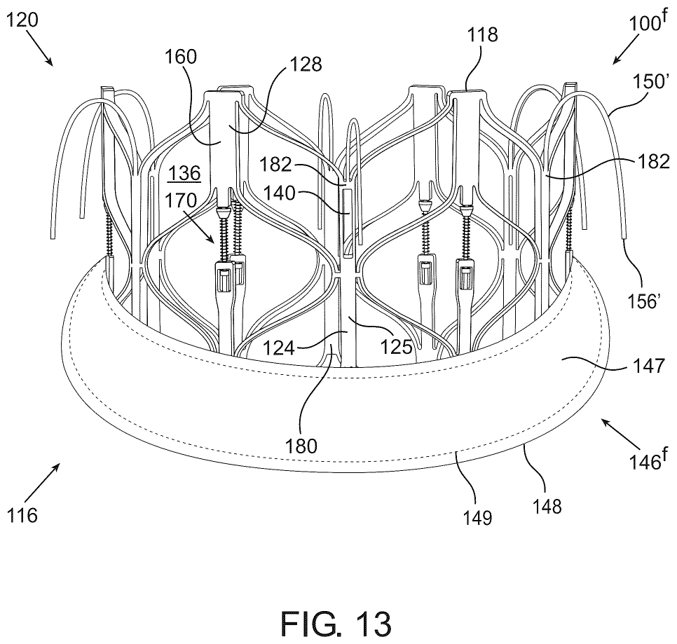

FIG. 13 shows another exemplary prosthetic valve 100′, which can be similar to other types of prosthetic valves 100 described above, except that it includes an outer skirt 146″ that comprises a scaling ring 147. The sealing ring 147 desirably is sized such that when the prosthetic valve 100′ is implanted in the native annulus, it completely covers any gap that may exist between the frame 104 and the native annulus.

The outer skirt 146′ can further include a flat base layer disposed around the frame 104 (in a similar manner to outer skirt 146ª illustrated in FIG. 2A) to which the sealing ring 147 is attached, or can be comprised solely of the sealing ring 147 without including a flattened base layer. The sealing ring 147 can be coupled (e.g., sutured) to the frame 104 and/or to an inner skirt of the prosthetic valve. In some implementations, as shown, the scaling ring 147 can be disposed around the valve inflow end 116.

The prosthetic valve 100^{f} further includes outflow support arms 150′, such as six support arms 150′ shown in the illustrated example, though any other number is contemplated. The prosthetic valve 100^{f} can be devoid of inflow support arms 150″, the role of which can be replaced by the scaling ring 147.

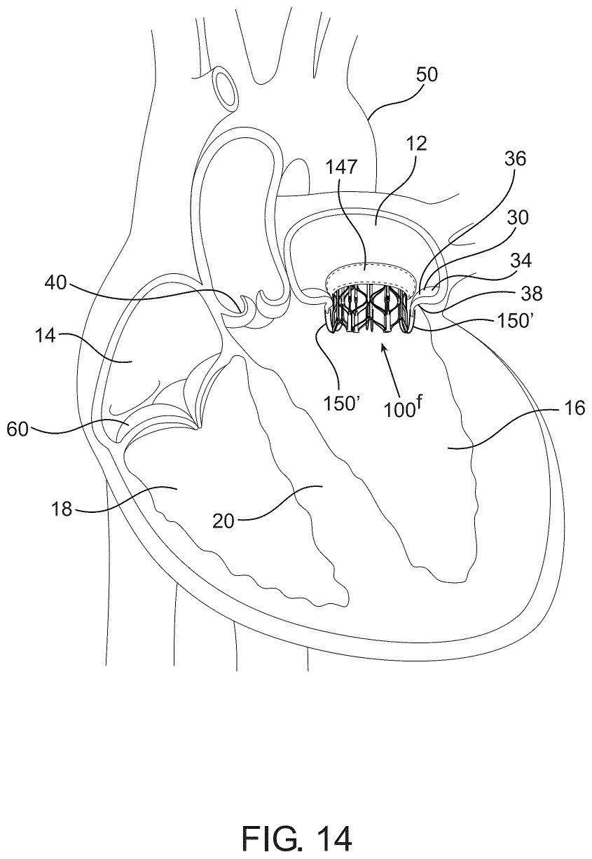

FIG. 14 depicts a prosthetic valve 100^{f} implanted in a native mitral valve 30. The prosthetic valve 100^{f} is particularly suitable for deployment in a larger native annulus, such as that of the native mitral valve 30, which can in some instances lack the sufficient anatomical structure to retain a typical prosthetic valve in place. This is because the outflow support arms 150′ abut the mitral ventricular abutment surface 38 to prevent migration of the prosthetic valve 100 toward the left atrium 12, while the sealing ring 147 abuts the mitral atrial abutment surface 36 to prevent migration of the prosthetic valve 100 toward the left ventricle 16. Furthermore, the increased diameter of the sealing ring 147 can provide adequate PVL sealing, even if the prosthetic valve 100^{f} is implanted in a range of native annuluses that can be greater in size than the prosthetic valve 100^{f} diameter.

In some implementation, such as in the illustrated example, the sealing ring 147 can be configured to abut the mitral atrial abutment surface 36, in a manner that allows the scaling ring 147 to completely cover any gaps or opening between the mitral valve 30 and the frame 104. The sealing ring 147 is preferably impervious to the flow of blood, allowing it to effectively block blood from flowing back into the left atrium 12 between outer surfaces of the prosthetic valve 100′ and the native tissue, ensuring that all, or substantially all, of the blood passes through the valvular structure 108 from the left atrium 12 to the left ventricle 16. In this manner, the sealing ring 147 can serve to better retain the prosthetic valve 100′ in place against migration toward the left ventricle 16, as mentioned above.

Alternatively, or additionally, the sealing ring 147 can be made of a relatively compressible or squeezable material, configured to have at least a portion thereof squeezed within the native annulus (e.g., the mitral annulus 32), such that the sealing ring's 147 outer surface can conform to irregularities around the native annulus and seal it.

In some implementations, the sealing ring 147 includes a textured outer surface, configured to promote tissue overgrowth or thrombosis, such that over time, such tissue overgrowth can improve PVL sealing against the native tissue.

In some examples, the sealing ring 147 can extend radially away from the frame 104 to a distance of at least 2 mm, or at least 5 mm, in a free or un-squeezed state thereof. In some examples, the scaling ring 147 can include a compressible or squeezable insert 149 and a cloth cover 148. For example, the squeezable insert 149 can be made of a silicone-based material, although other compressible materials can be used. The cloth cover 148 can be formed of any biocompatible fabric, such as, for example, polyethylene terephthalate or polyester fabric. In other implementations, the sealing ring 147 can be formed by rolling a flat sheet of cloth material to form a cylinder-like member.

While illustrated for use in a native mitral valve 30 in FIGS. 12 and 14, it is to be understood that the prosthetic valve 100, such as valve 100^{e} and/or 100^{f}, respectively, can be similarly implanted within a native tricuspid valve 60, a dilated aortic valve, or any other enlarged orifice. Utilization of the proposed prosthetic valve 100 (such as valve 100^{e} and/or 100^{f}) for implantation in such enlarged native valves or orifices can advantageously provide adequate anchoring against the native tissue, as well as improved PVL sealing when including a sealing ring 147, without requiring the aid of additional devices such as docking stations that can be alternatively used in such scenarios, thus simplifying the implantation procedure.

As mentioned above, conventional prosthetic valve may be appropriately sized for placement inside many native cardiac valves or orifices. However, with larger native valves, blood vessels (e.g., an enlarged aorta), grafts, etc., conventional prosthetic valves might be too small to secure into the larger implantation or deployment site. In this case, the prosthetic valve may not be large enough to sufficiently expand inside the native valve or other implantation or deployment site or the implantation/deployment site may not provide a good seat for the prosthetic valve to be secured in place.

FIG. 15 shows a cutaway view of a human heart with an exemplary docking station 90 positioned in the inferior vena cava 70. A docking station 90 that includes a valve seat 94 in which a prosthetic valve 96 is placed, can be generally used to supplement the function of a defective tricuspid valve 60 and/or to prevent too much pressure from building up in the right atrium 14. During systole, the leaflets of a normally functioning tricuspid valve 60 close to prevent the venous blood from regurgitating back into the right atrium 14. When the tricuspid valve 60 does not operate normally, blood can backflow or regurgitate into the right atrium 14, the inferior vena cava 70, the superior vena cava 72, and/or other vessels in the systolic phase. Blood regurgitating backward into the right atrium 14 increases the volume of blood in the atrium and the blood vessels that direct blood to the heart. This can cause the right atrium 14 to enlarge and cause blood pressure to increase in the right atrium 14 and blood vessels, which can cause damage to and/or swelling of the liver, kidneys, legs, other organs, etc. A prosthetic valve implanted in the inferior vena cava 70 and/or the superior vena cava 72 can prevent or inhibit blood from backflowing into the inferior vena cava 70 and/or the superior vena cava 72 during the systolic phase.

An exemplary docking station 90 illustrated in FIG. 15 can include a valve seat 94 and one or more retaining portions 92. The valve seat 94 provides a supporting surface for implanting or deploying a prosthetic valve 96 in the docking station 90 after the docking station 90 is implanted in the circulatory system. The retaining portion 92 helps retain the docking station 90 and the valve 96 at the implantation position or deployment site in the circulatory system. The retaining portion 92 can take a wide variety of different forms. In one exemplary implementation, the retaining portion 92 includes friction enhancing features that reduce or eliminate migration of the docking station 90. The friction enhancing features can take a wide variety of different forms. For example, the friction enhancing features can comprise barbs, spikes, texturing, adhesive, and/or a cloth or polymer cover with high friction properties on the retaining portions 92.

FIG. 15 illustrates one example of a docking station 90 and prosthetic valve 96 deployed in the inferior vena cava 70. However, it is to be understood that the docking station 90 and valve 96 can be deployed in any interior surface within the heart or a lumen of the body. When the heart is in the diastolic phase, as shown in FIG. 15, the prosthetic valve 96 opens. Blood flows from the inferior vena cava 70 and the superior vena cava 72, into the right atrium 14. The blood that flows from the inferior vena cava 70 flows through the docking station 90 and valve 96. Also, while in the diastolic phase, blood in the right atrium 14 flows through the tricuspid valve 60, and into the right ventricle 18.

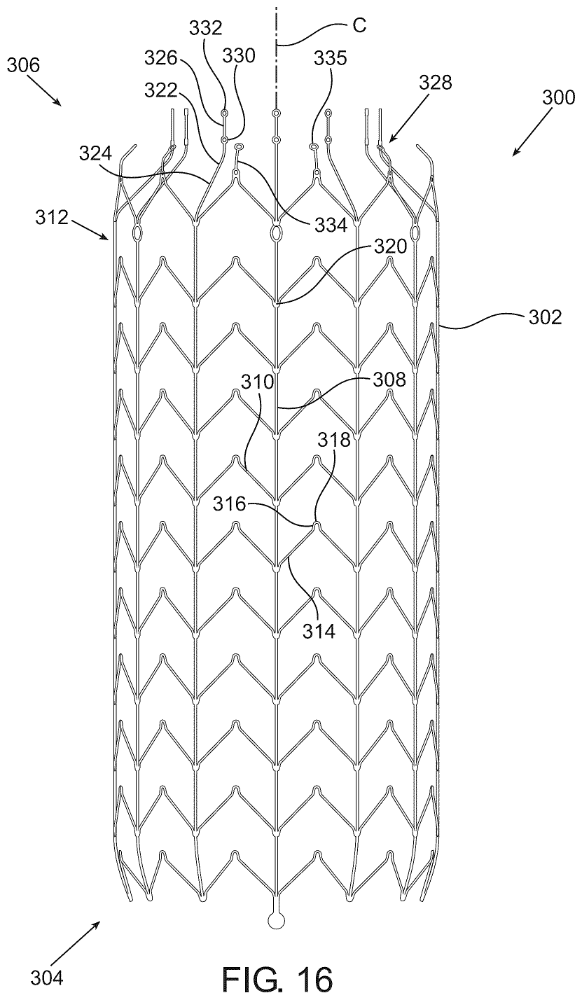

FIG. 16 illustrates an example of a radially collapsible and expandable docking frame 302 of a docking station 300 configured for implantation in a body lumen, such as in the inferior vena cava 70 or the superior vena cava 72 of the human heart. Only half of the total circumference of the frame is illustrated in FIG. 16 for clarity. The docking frame 302 can comprise an inflow end portion 304 and an outflow end portion 306. The docking frame 302 can comprise a plurality of longitudinal strut members 308 circumferentially spaced apart from each other around the docking frame 302. The docking frame can further comprise a plurality of rows of angled struts 310 arranged alternatingly in a zig-zag pattern. The rows of angled struts 310 can be axially spaced apart from each other along a longitudinal axis of the frame.

The angled struts 310 are arranged such that first end portions 314 of the struts are coupled to longitudinal strut members 308 at junctions 320, and second end portions 316 of the struts are coupled to second end portions of adjacent struts 310 to form “free” apices 318. The apices 318 can be arranged in circumferential rows, each row spaced apart from the preceding and succeeding rows along the longitudinal axis C. In the illustrated example, the free apices 318 are oriented in the direction of the outflow end portion 306, but the apices can also be oriented toward the inflow end portion 304. When oriented in the downstream/outflow direction, the apices 318 of the struts 310 can engage the tissue of the inferior vena cava 70 and reduce or prevent downstream displacement or migration of the docking frame 302 post-implantation. In certain embodiments, the orientation of the apices 318 in the downstream direction (and the lack of apices oriented in the upstream direction) can also facilitate proximal/upstream motion of the frame through the inferior vena cava 70, allowing recapture of the frame and/or retrieval/removal of the docking station from the patient in certain implementations.

As noted above, the docking station 300 can be radially collapsed to a collapsed or crimped configuration for delivery to the treatment site through a patient's vasculature. In certain implementations, the docking frame 302 can be made of a shape memory material, such as the nickel titanium alloy (Nitinol), Elgiloy, stainless steel, or combinations thereof, that allows the frame to be compressed to a reduced diameter for delivery in a delivery apparatus and then causes the frame to expand to its functional size inside the patient's body when deployed from the delivery apparatus. In certain examples, the docking frame 302 can be configured as a plastically-expandable or balloon-expandable frame adapted to be crimped onto an inflatable balloon or other expansion mechanism of a delivery apparatus and expanded to their functional size by. Such plastically expandable or ductile materials include nickel-chromium alloys, stainless steel, etc.

The outflow end portion 306 can comprise a plurality of struts 322 coupled to junctions 320 of an outflow row of struts 312. The struts 322 that can extend in a downstream direction, and can be angled radially inwardly toward the longitudinal axis C. More particularly, the struts 322 can comprise first portions 324 coupled to the junctions 320 and angled inwardly toward the longitudinal axis, and second portions 326 extending from the first portions 324 parallel, or substantially parallel, to the longitudinal axis C. The second portions 326 of the struts 322 can thereby define a valve-receiving portion or valve seat generally indicated at 328, which can be coaxial with the docking frame 302 and configured to receive a prosthetic valve. The valve seat 328 can have a diameter which is less than the diameter of the main body of the docking frame 302. The second portions 326 of the struts 322 can further comprise one or a plurality of apertures or openings, such as openings 330 and 332 spaced apart along the portions 326. In certain examples, the free end portions (e.g., at 332) of the struts 322 can define a downstream-most end of the frame.

In some examples, the struts 310 of the outflow row 312 can further comprise struts 334 extending from apices 318 and curving radially inwardly. The end portions of the struts 334 can comprise openings 335, and can define a diameter that is less than the diameter of the main body of the docking frame 302 and greater than the diameter of the valve seat 328.

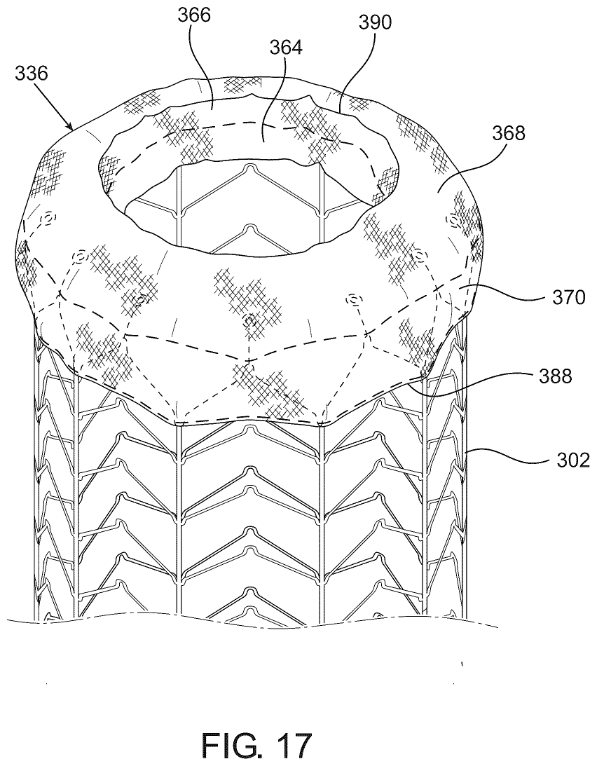

In certain examples, a scaling member can be disposed on the outflow end portion 306, such as the representative sealing member 336 shown in FIG. 17. In certain examples, the scaling member 336 can cover at least a portion of the exterior surfaces of struts 322, the struts 310 of the outflow row 312, and the struts 334. In certain examples, the sealing member 336 can comprise an inner portion disposed radially inwardly of the struts 322 and coupled to the struts 322 (e.g., by suturing), an outer portion covering outer surfaces of the struts 322, the struts 310 of the outflow row 312, and the struts 334, and an intermediate portion extending between the inner portion and the outer portion, although the scaling member may have any configuration that facilitates sealing between a prosthetic valve and the valve seat 328, and/or between the outer surface of the docking frame 302 and the surrounding anatomy at the outflow end portion of the frame. The sealing member 336 can be coupled/secured/attached to the docking frame 302 for example, by suturing through the various strut openings 319, 335, 330, 332, and/or the openings in apices 318 of the struts of the outflow row 312.

In certain examples, various components of the sealing member 336 can be made from any of various materials, such as woven or non-woven fabrics, polymeric laminate materials, composite materials, etc. For instance, the various portions 364-370 of the sealing member can comprise, for example, woven fabrics comprising any of a variety of synthetic/polymeric and/or natural fiber materials, such as polyethylene terephthalate (PET) fabric (e.g., DACRON®), polyester fabric, polyamide fabric, Nylon, polytetrafluoroethylene (PTFE), expanded polytetrafluoroethylene (ePTFE), ultra-high molecular weight polyethylene (UHMWPE) (e.g., DYNEEMA®), polypropylene, cotton, combinations thereof, etc. In certain examples, any or all of the portions of the sealing member 436 can also comprise a film including any of a variety of crystalline or semi-crystalline polymeric materials, such as polytetrafluorcthylene (PTFE), PET, polypropylene, polyamide, polyetheretherketone (PEEK), etc. In this manner, the scaling member 336 can be relatively thin and yet strong enough to allow it to be sutured to the frame 302, and to allow a prosthetic valve to be expanded against it, without tearing.

The first portion 364 can be positioned within the outflow end portion 306 of the frame 302. The central portion 366 can be positioned within the valve seat 328 defined by the strut members 322. The scaling member 336 can be folded over the outer aspect of the frame 302 such that the sealing member extends over or covers the struts 322, the struts 334, the valve seat 328, and covers the outflow row 312 of struts 310. Thus, the fourth portion 370 can be positioned against the radially outward surface of the frame 302, terminating at a scaling member inflow end 388 defined by the outer portion (i.e., fourth portion 370) of the scaling member, opposite to an outflow end 390 of the sealing member, defined as the portion of the scaling member disposed over the outflow end portion 306 of the frame 302.

Previously disclosed docking stations, including docking station 300 of the type described above with respect to FIGS. 15-17, include a valve seat (e.g., valve seat 328) defining an inner diameter configured to provide retention force when a prosthetic valve is deployed therein. Additional examples of docking stations can be found in International Application No. PCT/US2021/046207 and U.S. Publication No. 2019/0000615, which are incorporated by reference herein.

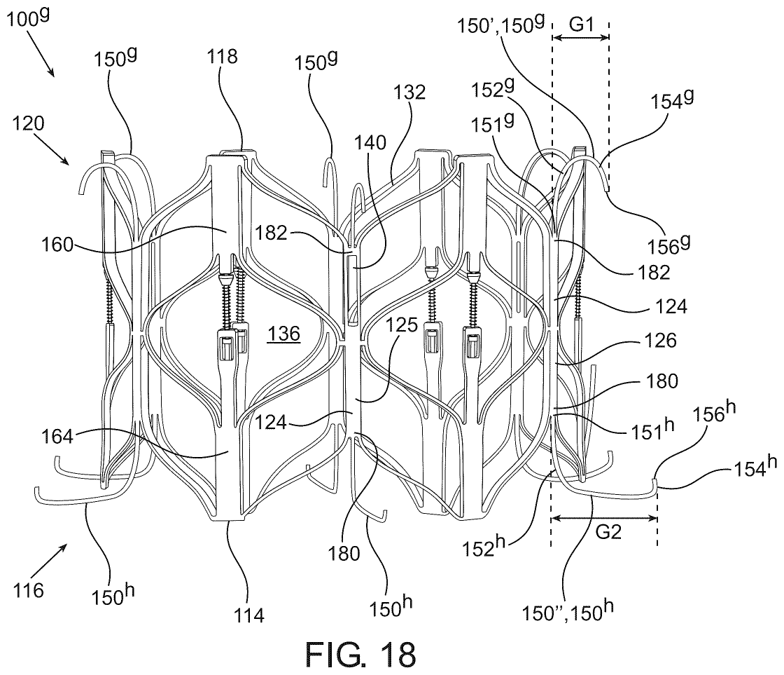

FIG. 18 shows an exemplary prosthetic valve 100^{g} that includes outflow support arms 150′ and inflow support arms 150″. A radial gap G can be defined as the radial distance between the tip 156 and the frame 104, and in particular, the tip 156 and the base 151 of the corresponding arm 150, in a free (i.e., unconstrained) state of the arm. In some implementations, the prosthetic valve 100^{g} includes differently sized outflow support arms 150^{g} and inflow support arms 150^{h}. The outflow support arms 1508 defined a radial gap G1, while the inflow support arms 150^{h} define a gap G2 that is greater than G1.

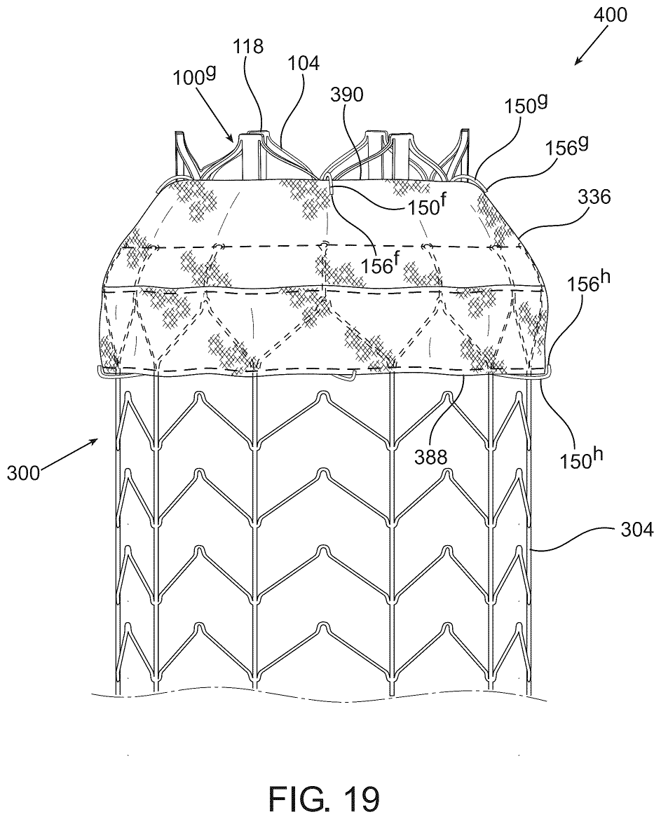

FIG. 19 shows a prosthetic assembly 400 that includes the prosthetic valve 100^{g} mounted in the docking station 300. While the valve seat diameter is configured to accommodate deployment of the frame 104 therein, the gaps G1 are sized to allow the outflow support arms 1508 to abut or rest over the sealing member outflow end 390, for example such that the first portions 1528 are disposed over the outflow end 390 while the tips 156^{g} can be disposed distal to the sealing member outflow end 390, over an outer surface of the sealing member 336. The greater gaps G2 are sized to allow the inflow support arms 150^{f} to abut or rest over the inflow end 388 of the sealing member 336, for example such that the first portions 152^{h} are disposed over the inflow end 388 while the tips 156^{h} can be disposed proximal to the inflow end 388, over an outer surface of the scaling member 336. This configuration advantageously prevents spontaneous or otherwise undesired axial displacement of the prosthetic valve 100 within the valve seat 328 of the docking station 300, thus providing additional retention force to the valve 100 within the docking station 300.

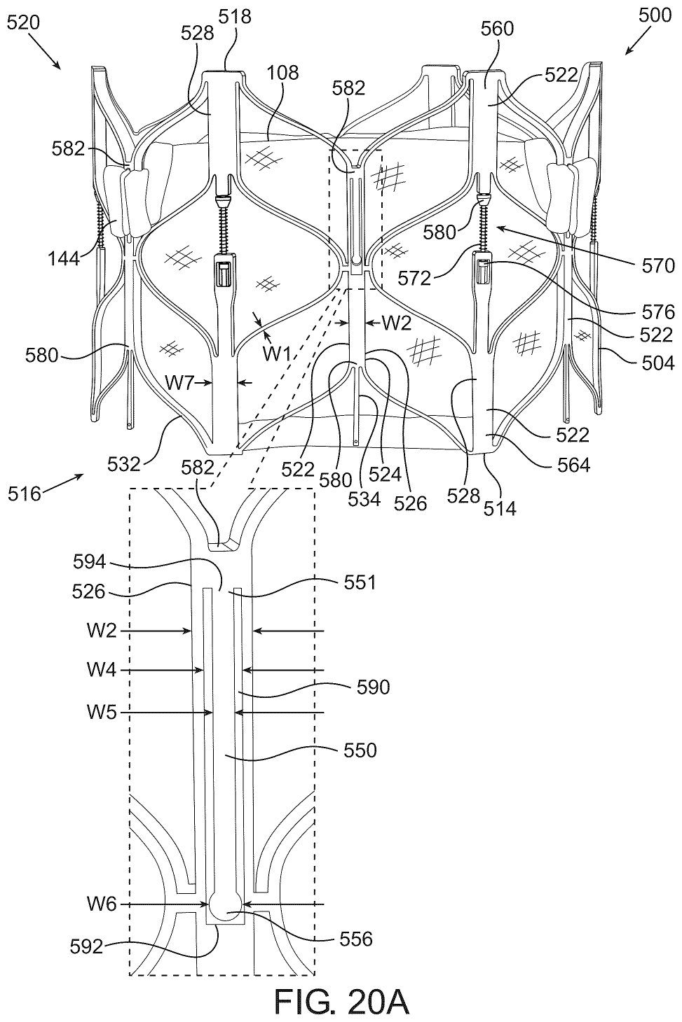

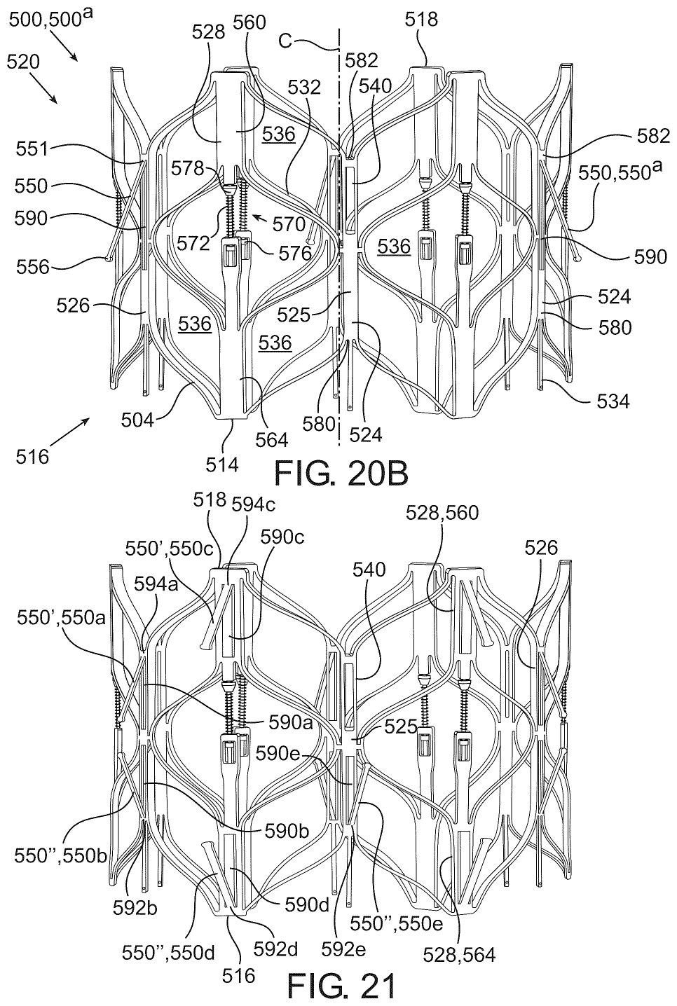

FIGS. 20A-20B illustrate another example of a prosthetic valve 500, which can be similar to prosthetic valve 100 described above with respect to FIGS. 2A-2D, with like numbers referring to like components, except that prosthetic valve 500 includes support arms 550 that extend from an end of corresponding openings 590 formed within a vertical posts 522, such as non-commissural support posts 526. Specifically, a mechanically expandable prosthetic valve 500 can include an annular frame 504 and a valvular structure 108 supported within and coupled to the frame 504, as shown in FIG. 20A. Though not illustrated with the skirt for sake of clarity, it is to be understood that a prosthetic valve 500 can further include any of the skirts described above, including skirt 146^{a} described above in conjunction with FIG. 2A, or skirt 146^{f} described above in conjunction with FIGS. 13-14.

The frame 504 has an inflow end 516 and an outflow end 520, and includes a plurality of support posts 524 and actuation posts 528 that can be arranged in an alternating manner along the circumference of the frame 504. The frame 504 further includes angled struts 532 extending circumferentially between adjacent vertical posts 522. Specifically, angled struts 532 circumferentially extend between support posts 524 and actuation posts 528 and interconnect the support posts 524 and actuation posts 528. The angled struts 532, support posts 524, and actuation posts 528, define cells 536 of the frame 504. As illustrated, the angled struts 532 can have a curved shape. As shown, the actuation posts 528 are arranged in pairs, each pair including an upper post member 560 and a lower post member 564 which can be axially aligned with each other, and each pair of actuation posts 528 can be connected, such as via angled struts 532, to a commissure support post 525 on one side thereof, and to a non-commissural support post 526 on the other side.

Support posts 524 can include commissure support posts 525, which include commissure windows 540 configured to support commissures 144 attached thereto, and non-commissural support posts 526, which include post openings 590. The commissure support posts 525 and non-commissural support posts 526 can be arranged in an alternating manner along the circumference of the frame 504. In the illustrated example, frame 504 includes a total of six support posts 524, three of which are commissure support posts 525 and three of which are non-commissural support posts 526.

One or more of the support posts 524 can further include cantilevered struts 534 extending to the inflow end 516 of the frame 504. In some examples, the cantilevered struts 534 can extend such that distal ends of the cantilevered struts 534 align with or substantially align with the inflow end 516 of the frame 504.

Each support post 524, including any commissure support posts 525 or non-commissural support post 526, extends between a post inflow end 580 which is closer to the inflow end 516 of the valve 500, and a post outflow end 582 which is closer to the outflow end 520 of the valve 500. Two angled struts 532 intersect with each support post 524 at a post inflow end 580, which is circumferentially disposed between two adjacent inflow apices 514, such that a corresponding cantilevered strut 534 can extend distally from the post inflow end 580. Similarly, two angled struts 532 intersect with each support post 524 at a post outflow end 582, which is circumferentially disposed between two adjacent outflow apices 518.

As further shown, each commissure support post 525 and each non-commissural support post 526 also intersects, at a middle portion thereof, with four additional angled struts 532 extending from adjacent upper post members 560 and lower post members 564 on both sides, resulting in each support posts 524, and specifically, each commissure support post 525 and each non-commissural support post 526, intersecting with a total of at least eight curved struts extending from adjacent actuation posts 528.

In one example, the frame 504 can be adjusted between a radially expanded configuration and a radially compressed configuration by deflecting the angled struts 532. In one example, the frame 504 (e.g., the posts and struts) can be made of biocompatible plastically-expandable materials that will allow the frame 504 to be adjusted between the radially expanded configuration and radially compressed configuration. Suitable examples of plastically-expandable materials that can be used in forming the frame 504 include, but are not limited to, stainless steel, cobalt chromium alloy, and/or nickel titanium alloy (which can also be referred to as “NiTi” or “nitinol”).

In some examples, one or more actuators 570 can be coupled to the actuation posts 528, and used to adjust the frame 504 between the radially expanded configuration and the radially compressed configuration. In one example, each actuation post 528 can include an upper post member 560 and a lower post member 564 (the terms “upper” and “lower” are relative to the orientation of the prosthetic valve 500 in FIGS. 20A-20B) aligned with the longitudinal axis of the valve and having opposing ends separated by a gap. The respective actuator 570 can be coupled to the post members 560, 564 and operable to increase or decrease the gap therebetween in order to radially compress or expand the frame 504. Angled struts 532 can converge with upper post members 560 to define outflow apices 518 at the outflow end 520. Angled struts 532 can similarly converge with lower post members 564 to define inflow apices 514 at the inflow end 516.

In one example, the actuator 570 can include an actuator rod 572 with an attached actuator head. In the example illustrated in FIGS. 20A-20B, the actuator rod 572 extends through or into the post members 560, 564 and across the gap therebetween. In the example illustrated in FIGS. 20A-20B, the actuator rod 572 is inserted into the upper post member 560 from the outflow end 520, and the actuator head (hidden from view in FIGS. 20A-20B) can be disposed or retained at the outflow apex 518 of the upper post member 560.

In some examples, the actuator rod 572 is externally threaded. As illustrated in FIGS. 20A-20B, the lower post member 564 can include a nut 576 with an internal thread to threadedly engage the actuator rod 572. In this case, the actuator rod 572 can be axially translated by rotating the actuator rod 572 relative to the nut 576. In some examples, the actuator rod 572 can be freely slidable relative to the upper post member 560. In other examples, the actuator rod 572 can threadedly engage the upper post member 560.

In one scenario, the actuator rod 572 can be rotated in a first direction to move the upper post member 560 towards the lower post member 564 and thereby decrease the size of the gap therebetween, which can have the effect of radially expanding the frame 504. In another scenario, the lower post member 564 may be held steady while the actuator rod 572 is rotated in a second direction to move the upper post member 560 away from the lower post member 564 and thereby increase the size of the gap therebetween, which can have the effect of radially compressing the frame 504.

The actuator rod 572 also can include a stopper 578 (e.g., in the form of a nut, washer or flange) disposed thereon. The stopper 578 can be disposed on actuator rod 572 such that it sits within the gap therebetween. Further, the stopper 578 can be integrally formed on or fixedly coupled to the actuator rod 572 such that it does not move relative to the actuator rod 572. Thus, the stopper 578 can remain in a fixed axial position on the actuator rod 572 such that it moves in lockstep with the actuator rod 572.

When the actuator rod 572 is rotated in a direction configured to collapse the prosthetic valve, the stopper 578 moves toward the outflow end 520 of the frame until the stopper 578 abuts the inflow end of the upper post member 560. Upon further rotation of the actuator rod 572, the stopper 578 can apply a proximally directed force to the upper post member 560 to radially compress the frame 504. Specifically, during crimping/radial compression of the prosthetic valve 500, the actuator rod 572 can be rotated in a direction that causes the stopper 578 to push against (i.e., provide a proximally directed force to) the inflow end of the upper post member 560, thereby causing the upper post member 560 to move away from the lower post member 564, and thereby axially elongating and radially compressing the prosthetic valve 500.

In an alternative implementation, some of the actuator rods 572 can be rotated in one direction while the other actuator rods 572 are rotated in an opposite direction simultaneously to cither radially expand the frame or radially compress the frame. This counter-rotation of the actuator rods can be used to help reduce the likelihood of the entire frame 504 rotating about its central longitudinal axis during rotation of the actuator rods 572 about their respective axes (e.g., when radially expanding the frame 504).

Each angled strut 532 can have a width W1 in the lateral or circumferential direction, which is less than the width of any of the vertical posts. Specifically, each support post 524 can have a width W2, which is greater than the width W1 of the angled struts. Since commissure support posts 525 can include commissure window 540, and non-commissural support posts 526 can include post openings 590, the width W2 can be selected to accommodate commissure windows 540 and post openings 590 in the respective support posts 524. The actuation posts 528, including any upper post member 560 and lower post member 564, define a width W7, which can be similar to, or different from, the width W2, but will also be greater than the width W1 of the angled struts. Actuation posts 528 will be relatively wider than angled struts as they include bores through which actuation rods 572 can extend. One or more of the actuation posts 528 can also include a post opening 590, in which case the width W7 can be selected to accommodate post openings 590 in the respective actuation posts 528.

Each post opening 590 can extend from an opening inflow end 592, which is the end closer to the post inflow end 580, and an opening outflow end 594, which is closer to the post outflow end 582. The post opening 590 can define a width W4 in the lateral or circumferential direction, which is less than the width W2, such that the difference between W2 and W4 defines the width of the sidewalls of a post opening 590 comprised in a support post 524. In some examples, the width W2 is greater than the width W4 by at least 10%. In some examples, the width W2 is greater than the width W4 by at least 20%. In some examples, the width W2 is greater than the width W4 by at least 30%. In some examples, the width W2 is greater than the width W4 by at least 50%. In some examples, the width W2 is greater than the width W4 by at least 70%. In some examples, the width W2 is at least two times greater than the width W4.

In a similar manner, the width W4 can be less than the width W7, such that the difference between W7 and W4 defines the width of the sidewalls of a post opening 590 comprised in an actuation post 528. In some examples, the width W7 is greater than the width W4 by at least 10%. In some examples, the width W7 is greater than the width W4 by at least 20%. In some examples, the width W7 is greater than the width W4 by at least 30%. In some examples, the width W7 is greater than the width W4 by at least 50%. In some examples, the width W7 is greater than the width W4 by at least 70%. In some examples, the width W7 is at least two times greater than the width W4.

A prosthetic valve 500 further includes one or more support arms 550 extending from vertical posts 522, such as support posts 524 and/or actuation posts 528. Unlike support arms 150 of prosthetic valve 100 described above, a support arm 550 does not extend from an end of a support post, such as a post outflow end or a post inflow end, but rather from an end of a post opening 590. In the examples illustrated in FIGS. 20A-20B, a prosthetic valve 500ª is shown to include three support arms 550ª extending from opening outflow ends 594 of post opening 590 formed in outflow portions of non-commissural support posts 526. While three support arms 550 are shown in the illustrated example, it is to be understood that any other number of support arms is contemplated, such as a single support arm, two support arms, or more than three support arms.

A support arm 550 can be cut from a corresponding vertical post 522, wherein the cutting edges form the resulting post opening 590. Specifically, a support arm 550 can be formed by cutting an appropriate longitudinal portion (e.g., laser cutting) through the thickness of the vertical post 522 along a circumference that spans three sides the arm 550, while leaving one side of the arm uncut or intact. The uncut end of the arm 550 forms the base 551 of the arm 550, at which it integrally extends from the corresponding uncut end of the post opening 590. In the illustrated example, an uncut base 551 of the arm 550 is shown at the opening outflow end 594, such that the support arm 550 distally extends from the opening outflow end 594 toward the opening inflow end 592, terminating with a free-ended cut tip 556 in the vicinity of the opening inflow end 592.

Since the support arm 550 is cut from the vertical post 522, it is aligned with the respective resulting post opening 590, such that the length of the arm 550 (defined between the base 551 and tip 556) is equal to or shorter than the length of the post opening 590 (defined between the opening inflow end 592 and opening outflow end 594). The arm can define a width W5, which can be similar to or less than the width W4 of the post opening 590 it is cut from. As described above with respect to tips 156 of support arms 150, the tip 556 is preferably atraumatic to avoid damaging abutment surfaces it is configured to contact or rest on. For example, the tip 556 may have a smooth contact surface that may be flattened or curved and is not configured to penetrate the tissue it is configured to contact and/or rest on. The tip 556 can be covered in some implementations. In some examples, the tips 556 can be configured to be flexible to allow for reduction of possible trauma to the tissue upon contact. In the illustrated example, the tip 556 is shown to be rounded, optionally formed to define a diameter or width W6 which can be slightly greater than the width W5 of the rest of the arm 550. However, since any portion of the support arm 550 is cut from the respective support post, even for a wider tip 556, the width W6 will be similar to or less than the width W4 or the corresponding post opening 590. It is to be understood that any of the support arms 150 disclosed hereinabove, can be optionally equipped with similarly formed wider tips 156.

After being cut from the respective vertical post, the support arm 550 can be shape-set (e.g., heat set) to deflect at an angle radially away from the rest of the frame 504, resulting in the tip 556 being biased radially away and spaced away from the frame 504 (or from the respective post opening 590) in a free state of the support arm 550, as shown in FIG. 20B for example.

As mentioned, a prosthetic valve 500 can include any number of support arms 550. Thus, the plural use of the term “support arms 550” is not meant to be limiting, and may refer to implementation in which a single support arm 550 is included. For example, a prosthetic valve 500 can include any number of support arms, such as three support arms as shown in FIGS. 20A-20B, more or less than three arms, or even a single support arm 550 that can extend, for example, from an opening outflow end 594 of one of the non-commissural support posts 526. FIG. 21 shows exemplary support arms 550 that can extend from post openings 590 formed in various vertical posts 522.

Support arms 550 can extend from support posts 524, as shown for support arms 550a and 550b extending from non-commissural support post 525 and support arms 550c extending from commissure support posts 525. Support arms 550 can similarly extend from actuation posts 528, as shown for support arms 550c extending from upper post members 560 and support arms 550d extending from lower post members 564.

As shown in FIG. 21, a prosthetic valve 500 can include outflow support arms 550′ outflow support arms 550′ extending downwardly from post openings 590 formed in upper portions of vertical posts 522 (i.e., toward the inflow end 516), and inflow support arms 550″ extending upwardly from post openings 590 formed in lower portions of vertical posts 522 (i.e., toward the outflow end 520). Upper post openings 590 are formed closer to respective post outflow ends 582, as shown for post openings 550a, or closer to respective outflow apices 518, as shown for post opening 590c, in which case the support arms 550 integrally extend from the respective opening outflow ends 594. Lower post openings 590 are formed closer to respective post inflow ends 580, as shown for post openings 550b and 550c, or closer to respective inflow apices 516, as shown for post opening 590d, in which case the support arms 550 integrally extend from the respective opening inflow ends 592.

Outflow support arms 550′ will generally extend only from non-commissural support posts 526 or lower post members 564, since commissure support posts 525 include commissure windows 540 at this level of the frame 504. However, inflow support arms 550″ can extend from either type of vertical posts, including lower post members 564, non-commissural support posts 526, and/or commissure support posts 525. Any combination of outflow support arms and/or inflow support arms described above with respect to prosthetic valve 100, can be implemented in prosthetic valve 500, with the exception of outflow support arms 550′ that cannot extend from commissure support posts 525. It is to be understood that the various types and positions of support arms 550a, 550b, 550c, 550d and 550e are illustrated together in FIG. 21 for the sake of illustration and not limitation, and that any type and position of support arms can be used alone or in combination with any other type or position of the arms.

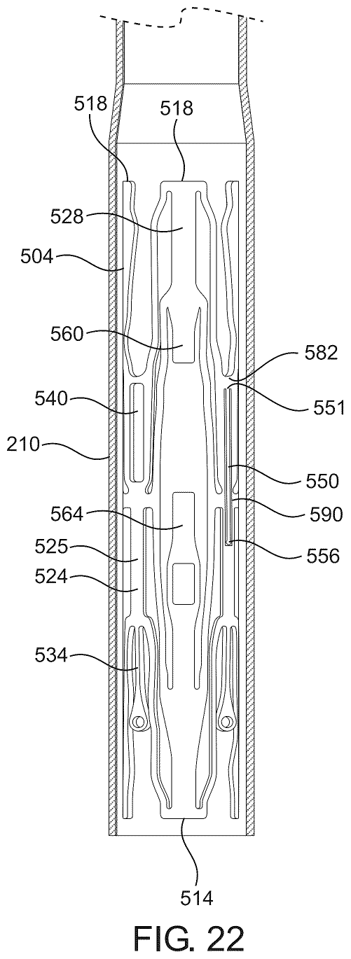

FIG. 22 shows a prosthetic valve 500 of FIGS. 20A-20B with self-expandable support arms 550 retained in an undeployed state within a capsule 210. While illustrated to be retained in a crimped state within a capsule 210, it is to be understood that the same configuration applies to a delivery apparatus that does not necessarily include a capsule, in which case the valve 500 can be similarly retained within a distal portion of a shaft of the delivery apparatus, such as the outer shaft 208. The actuator assemblies 220, which can be coupled to the prosthetic valve 500 during delivery, as well as actuators 570 and soft components of the valve 500, are removed from view in FIG. 22 for clarity.

As shown, during delivery, the support arms 550 can be pressed radially inward into their respective post opening 590. This configuration can be advantageous over other types of arms which are folded in a crimped configuration between the frame and the capsule, since the arms 550 can be completely flush with the outer surface of the rest of the frame 504 (and in particular, an outer surface of the respective support post), without affecting overall crimped profile of the valve during delivery. When the support arms 550 are freed from the capsule 210 during deployment of the prosthetic valve 500, they spring outwardly to their pre-formed shape, as shown in FIG. 20B.

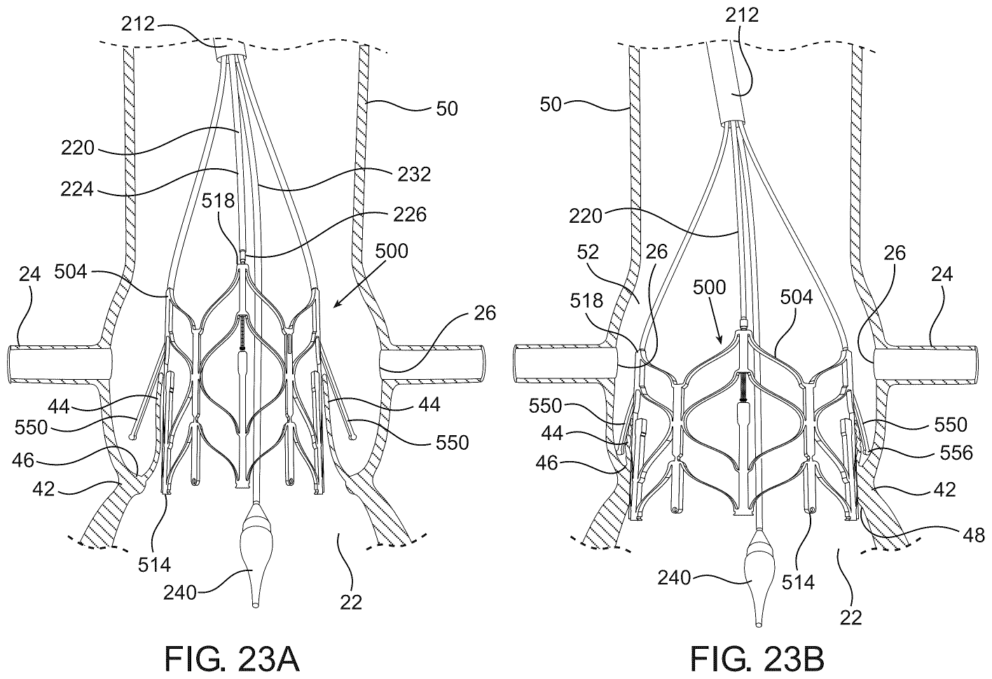

FIG. 23A-23B show stages of an exemplary method for implantation of a prosthetic valve 500^{a} of the type shown in FIGS. 20A-20B within a native aortic annulus 42. For simplicity, soft components, such as a valvular structure or a skirt, are not shown. The prosthetic valve 500 can be coupled to a delivery apparatus 202, which can be used to deliver, position, and secure the prosthetic valve 500 in a native heart valve annulus. In the illustrated implantation procedure, the prosthetic valve 500 is implanted in a native aortic annulus 42 using a transfemoral delivery approach. In other examples, the prosthetic valve 500 can be implanted at other locations (e.g., a mitral valve, a tricuspid valve, and/or a pulmonary valve), within previously-implanted prosthetic valve, and/or using other delivery approaches (e.g., transapical, transaortic, transseptal, etc.).

The prosthetic valve 500 can be releasably coupled, as described above, to the actuator assemblies 220 of delivery apparatus 202, and advanced in a compressed state through the patient's vasculature toward the site of implantation (e.g., the aortic annulus). Upon reaching the site of implantation, the prosthetic valve 500 can be deployed by pushing it distally out of the capsule 210 and/or outer shaft 208, or by proximally pulling the capsule 210 and/or outer shaft 208 relative to the prosthetic valve 500, which allows the support arms 550 to spring out radially outward, distancing the tips 556 from the corresponding post opening 590 and the rest of the frame 504 as described above with respect to FIG. 22, for example. The actuation assemblies 220 can then be utilized, as described above, to radially expand the prosthetic valve 500, at least to a partially expanded diameter, as shown in FIG. 23A.

As mentioned above with respect to mechanically expandable valve 100, the feasible controlled gradual expansion of a mechanically expandable prosthetic valve 500 can be taken advantage of for properly positioning the support arms 550 in a desired orientation, relative to the native anatomy. For example, as shown in FIG. 23A, actuator assemblies 220 can be utilized to partially expand the prosthetic valve 500 to a partially expanded diameter, which is greater than the crimped diameter but less than the final functional diameter, such that the frame 504, and optionally even the support arms 550, are not yet immovably pressed within the native annulus. This allows the prosthetic valve 500 to be angularly oriented to a desired angular orientation, for example with respect to the aortic leaflets 44.

As shown in FIG. 23A, when the prosthetic valve 500 is deployed within the native aortic valve 40, the support arms 550 can be positioned over and/or around the aortic leaflets 44. As shown in FIGS. 20A-20B, a prosthetic valve 500 can include a total of six support posts 524, three of which are commissure support posts 525 to which commissures 144 are coupled, and three of which are non-commissural support posts 526 disposed between the commissure support posts 525. In one configuration, the prosthetic valve 500 can include one or more (e.g., three as in the illustrated examples) support arms 550 integrally formed with non-commissural support posts 526 and extending from opening outflow ends 594 of corresponding post opening 590, while the commissure support posts 525 remain devoid of support arms. When implanted in a native aortic valve 40, it may be desired to position the support arms 550 over the native aortic leaflets 44 between the native commissures, such that the tips 556 can abut aortic root abutment surface 46 instead of contacting the native commissures.

The term “integral” or “integrally formed”, as used herein, refers to a construction of a component that does not include any welds, fasteners, adhesives or other means for securing separately formed pieces of material to each other. For example, an integrally formed support arm 550 is formed directly as a partially cut portion of a vertical post 522, rather than being separately formed and subsequently attached to the support post.

The prosthetic valve 500 can be deployed out of the capsule at a position which is proximal to the native aortic leaflets 44, allowing the support arms 550 to spring radially outwards, after which the prosthetic valve 500 can be distally advanced to position the native aortic leaflets 44 between the frame 504 and the support arms 550. Partial expansion of the prosthetic valve 500 can be performed upon deployment, prior to axial advancement, and/or during and/or after axial advancement of the valve 500. In the partially expanded diameter of the prosthetic valve 500, shown in FIG. 22A, it can be angularly reoriented, if needed, to align the commissures 144 of the prosthetic valve 500 with the native commissures between the native aortic leaflets 44.

Further expansion of the prosthetic valve 500 to the functional size, which can be optionally accompanied by further axial displacement relative to the native aortic annulus 42, serves to anchor the prosthetic valve 500 in position, wherein the support arms 550 may press against the native annulus for migration resistance. For example, the tips 556 can rest over the aortic root abutment surface 46, so as to resist unintentional distally oriented migration of the prosthetic valve 500 toward the left ventricle 16.

In some cases, the support arms 550 can extend around native leaflets and potentially grasp the native leaflets, as shown in FIG. 23B. For example, when the prosthetic valve 100 is further expanded by the actuator assemblies 220 from the partially expanded diameter shown in FIG. 23A to the desired functional diameter shown in FIG. 23B, the support arms 550 can be pressed between the surrounding aortic wall and the expanded frame 504, grasping portions of the native aortic leaflets 44 between the support arms 550 and the frame 504. The frame 504 and at least some portion of the support arms 550, such as the tips 556, can approximate each other in such procedures, optionally pinching the native leaflets 44 therebetween. Since the support arms 550 are aligned with corresponding post opening 590, portion of the native leaflet tissue can be pressed by the arms 550 into the post opening 590, which can improve securement of the prosthetic valve 500 to the leaflets 44. In some cases, the native leaflets 44 can be bunched up between the support arms 550 and the rest of the frame 504. This can also provide for greater securement of the prosthetic valve 500 to the leaflets 44, which may be specifically advantageous in some pathologies, such as Aortic Insufficiency, in which the native leaflets 44 do not include sufficient internal calcifications to warrant proper retaining force of a prosthetic valve pressed there-against. In alternative implementations, the support arms 550 do not necessarily hook the native leaflets.

While optional angular orientation of the prosthetic valve 500 is described above, in a partially expanded state thereof, it is to be understood that in alternative implementations, as described with respect to prosthetic valve 100, active angular orientation may not be required, relying on the natural tendency of the support arms 550 to slip along the native leaflets 44 to a position between the native commissures, during continuous expansion of the valve 500. In such implementations, the mechanical expansion mechanism of a mechanically expandable prosthetic valve 500 is still advantageous in that it allows sequential controlled expansion of the valve 500 at a rate that will allow the support arms 550 to be self-oriented to the proper positions along the native aortic leaflets 44, whereas abrupt expansion of conventional self-expandable valves, for example, may result in a less desirable outcome with respect to the position of the support arms 550 relative to the native anatomy.

Utilization of support arms 550 engaged with portions of the native tissue, such as annular abutment surfaces and/or native leaflets, can advantageously prevent or reduce the prevalence of axial movement of the prosthetic valve 500 in a direction opposite to the side the arms 550 extend from, and can also help ensure that the prosthetic valve 500 is perpendicular to the annulus and prevents undesirable “rocking” or tilting of the frame. By engaging the native annulus, and optionally the native leaflets, the support arm can assist in more evenly distributing the load to achieve equilibrium, thus providing a more robust implantation.

In some implementations, the radial gap formed between the tips 556 and the post opening 590 is configured to allow the support arms 550 to cover the native aortic leaflets 44 during gradual controlled expansion of the prosthetic valve 500. When further utilized to accommodate native aortic leaflets 44 therein, the native leaflets can be folded, for example by first arm portions 552 of the support arms 550, toward the native aortic annulus 42, distancing them away from the coronary arteries 24 to avoid obstruction of the ostia of the coronary arteries 24. Native leaflets bunched up within support arms 550 can also improve PVL scaling around the frame 504.

After reaching the final functional expanded diameter, the actuator assemblies 220 can be uncoupled from the actuators 570 and the delivery apparatus 202 can then be withdrawn from the patient's body, leaving the prosthetic valve 500 within the aortic annulus 42 to regulate blood flow from the left ventricle 16 into the aorta 50. While illustrated for use in a native aortic valve 40 in FIGS. 23A-23B, it is to be understood that a mechanically expandable prosthetic valve 500 with support arms 550 can be similarly implanted within a native tricuspid valve 60, a native mitral valve 30 valve, or any other orifice.

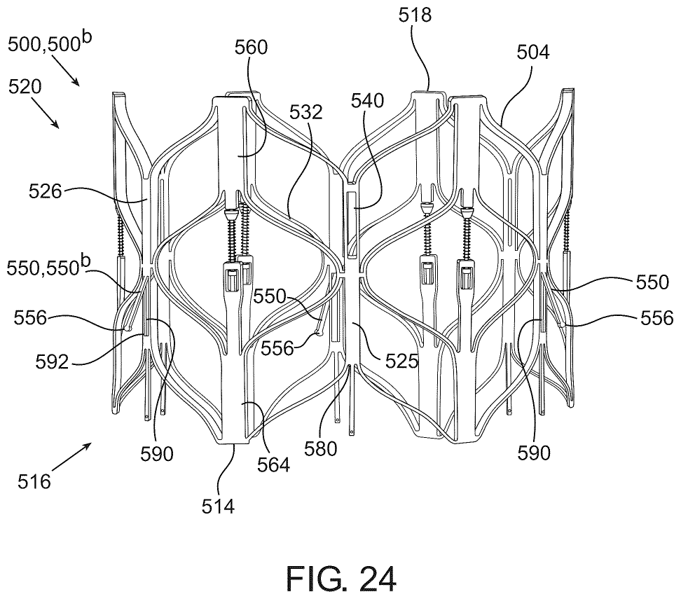

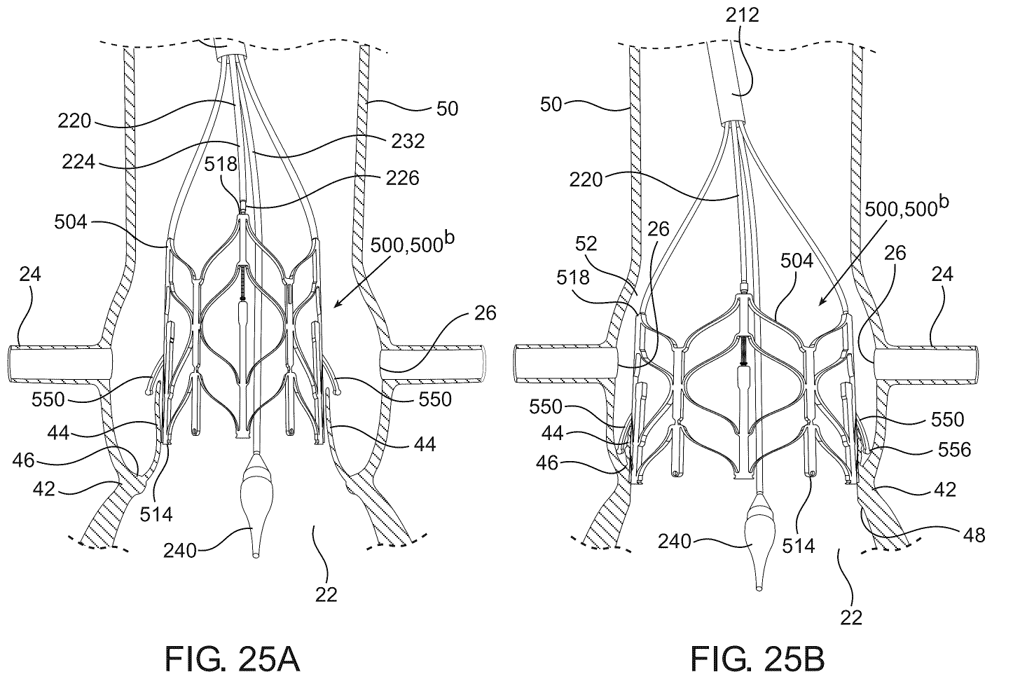

FIG. 24 illustrates another exemplary prosthetic valve 500^{b}, which can be similar to prosthetic valve 500^{a}, except for including support arms 550^{b} that extend from opening outflow ends 594 of post opening 590 formed in inflow portion of vertical posts 522, such as non-commissural support posts 526. FIGS. 25A-25B show stages of an exemplary method for implantation of a prosthetic valve 500^{b} of the type shown in FIG. 24 within a native aortic annulus 42, which can be similar to the method described above in conjunction with FIG. 23A-23B, except that the lower (i.e., more distal) position of the support arms 550^{b} is adapted to allow better positioning of a prosthetic valve 500h in a higher position relative to the annulus 42 (such as for supra-annular implantation procedures).

While support arms 550^{a} are shown to extend from post opening 590 formed at the outflow portion of vertical posts 522, and support arms 550^{b} are shown to extend from post opening 590 formed at the inflow portion of vertical posts 522, it is to be understood that the position in which post openings 590 are formed, with corresponding support arms 550 extending therefrom, can be along any other portion of the vertical post 522, including at a mid-portion of the vertical post. Moreover, while three support arms 550^{b} extending in a distal direction from opening outflow ends 594 of post opening 590 formed in inflow portions of non-commissural support posts 526 are illustrated, it is to be understood that any number of distally-oriented support arms 550 can extend from opening outflow ends 594 of post openings formed in any other type of vertical posts, such as location similar to those illustrated in FIG. 21 for posts openings 590b and 590e along inflow portions of lower posts members 564 and commissure support posts 525, respectively.

While FIGS. 20A-20B illustrate an exemplary prosthetic valve 500^{a} with distally-extending support arms 550^{a} extending solely from opening outflow ends 594 of post opening 590 formed in outflow portions of vertical posts 522, and FIG. 24 illustrates an exemplary prosthetic valve 500^{b} with distally-extending support arms 550^{b} extending solely from opening outflow ends 594 of post opening 590 formed in inflow portions of vertical posts 522, it is to be understood that any vertical posts 522 can include more than one distally extending support arm 550, each extending from an opening outflow end 594 of post openings formed at different axial positions along the vertical posts. For example, at least one non-commissural support post 526 can include one support arm 550^{a} distally extending from an opening outflow end 594 of a post opening 590 formed in its outflow portion, and another support arm 550^{b} distally extending from an opening outflow end 594 of a post opening 590 formed in its inflow portion. Such a combination can allow the prosthetic valve 500 to be axially positioned at any suitable position within the patient's annulus, depending on patient-specific native anatomy, with the appropriate set of support arms disposed over the native leaflets and clamped thereover.

While some types of a prosthetic valves 500 is illustrated in FIGS. 23A-23B and 25A-25B for implantation in an aortic annulus, it is to be understood that prosthetic valve 500 can be implanted in any other native orifice or annulus, including according to the implantation procedures and configurations described above in conjunction with any of FIGS. 12 and 14, and can be mounted within a docking station 300 in a similar manner to that described above in conjunction with FIGS. 18-19, mutatis mutandis.

### Some Examples of the Disclosed Technology

Some examples of the above-described technology are enumerated below. It should be noted that one feature of an example in isolation or more than one feature of the example taken in combination and, optionally, in combination with one or more features of one or more further examples are further examples also falling within the disclosure of this application.
Example 1. A prosthetic valve, comprising:
an annular frame movable between a radially compressed state and a radially expanded state, the frame comprising:
one or more pairs of actuation posts, each pair comprising an upper post member and a lower post member;
a plurality of support posts, wherein each support post extends between a post inflow end and an opposite post outflow end, and wherein the plurality of support posts comprises:
a plurality of commissure support posts, wherein each commissure support post comprises a commissure window; and
a plurality of non-commissural support posts, wherein each non-commissural support post is devoid of a commissure window;
a plurality of angled struts extending circumferentially between adjacent actuation posts and support posts and interconnecting the actuation posts and the support posts; and
one or more actuators coupled to the actuation posts, the one or more actuators configured to adjust the frame between the radially compressed state and the radially expanded state; and
a valvular structure mounted within the frame and comprising a plurality of leaflets configured to regulate flow through the prosthetic valve; and
at least one support arm extending from the post outflow end or the post inflow end of at least one of the support posts, wherein the support arm terminates at a free-ended tip which is biased radially away from the corresponding support post.
Example 2. The prosthetic valve of any example herein, particularly example 1, wherein two of the angled struts intersect with each post inflow end, and another two of the angled struts intersect with each post outflow end.
Example 3. The prosthetic valve of any example herein, particularly example 1 or 2, wherein each support post intersects with at least eight angled struts extending from adjacent actuation posts.
Example 4. The prosthetic valve of any example herein, particularly any one of examples 1 to 3, wherein the commissure support posts and the non-commissural support posts are arranged in an alternating manner along the circumference of the frame.
Example 5. The prosthetic valve of any example herein, particularly any one of examples 1 to 4, wherein the plurality of commissure support posts comprises three commissure support posts, and wherein the plurality of non-commissural support posts comprises three non-commissural support posts.
Example 6. The prosthetic valve of any example herein, particularly any one of examples 1 to 5, wherein the support arm is made of a shape-memory material.
Example 7. The prosthetic valve of any example herein, particularly example 6, wherein the shape-memory material comprises Nitinol.
Example 8. The prosthetic valve of any example herein, particularly any one of examples 1 to 7, wherein the width of the support post from which the support arm extends is greater than the width of any of the angled struts.
Example 9. The prosthetic valve of any example herein, particularly example 8, wherein the width of the support post is at least two times greater than the width of the angled struts.
Example 10. The prosthetic valve of any example herein, particularly example 8, wherein the width of the support post is at least three times greater than the width of the angled struts.
Example 11. The prosthetic valve of any example herein, particularly any one of examples 8 to 10, wherein the width of the support arm is less than the width of the support post it extends from.
Example 12. The prosthetic valve of any example herein, particularly example 11, wherein the width of the support arm is 70% or less than the width of the support post.
Example 13. The prosthetic valve of any example herein, particularly example 11, wherein the width of the support arm is 50% or less than the width of the support post.
Example 14. The prosthetic valve of any example herein, particularly example 11, wherein the width of the support arm is 30% or less than the width of the support post.
Example 15. The prosthetic valve of any example herein, particularly any one of examples 1 to 14, wherein the support arm is circumferentially deflected relative to the frame, such that the tip is circumferentially offset from the corresponding support post.
Example 16. The prosthetic valve of any example herein, particularly any one of examples 1 to 15, wherein the support arm is twisted.
Example 17. The prosthetic valve of any example herein, particularly example 16, wherein the support arm comprises two twists.
Example 18. The prosthetic valve of any example herein, particularly any one of examples 1 to 17, wherein the tip is atraumatic.
Example 19. The prosthetic valve of any example herein, particularly example 18, wherein the tip is rounded.
Example 20. The prosthetic valve of any example herein, particularly any one of examples 1 to 19, wherein the support arm comprises a first arm portion extending from a base of the support arm at the end of the corresponding support posts, and a second arm portion continuously extending from the first arm portion to the tip.
Example 21. The prosthetic valve of any example herein, particularly example 20, wherein the first arm portion is C shaped.
Example 22. The prosthetic valve of any example herein, particularly example 20, wherein the first arm portion is L shaped.
Example 23. The prosthetic valve of any example herein, particularly any one of examples 1 to 22, wherein the at least one support arm comprises a plurality of support arms.
Example 24. The prosthetic valve of any example herein, particularly example 23, wherein the plurality of support arms comprises outflow support arms extending from the post outflow ends of corresponding support posts.
Example 25. The prosthetic valve of any example herein, particularly example 24, wherein all of the outflow support arms extend solely from non-commissural support posts.
Example 26. The prosthetic valve of any example herein, particularly example 24 or 25, wherein the plurality of outflow support arms comprises three outflow support arms.
Example 27. The prosthetic valve of any example herein, particularly example 23 or 24, wherein the plurality of support arms comprises inflow support arms extending from the post inflow ends of corresponding support posts.
Example 28. The prosthetic valve of any example herein, particularly example 27, wherein at least one of the inflow support arms extends from a commissure support post.
Example 29. The prosthetic valve of any example herein, particularly example 23, wherein the plurality of support arms comprises at least one outflow support arm extending from the post outflow end of at least one of the support posts, and at least one inflow support arm extending from the post inflow end of at least one of the support posts.
Example 30. The prosthetic valve of any example herein, particularly example 29, wherein the at least one outflow support arm comprises a plurality of outflow support arms.
Example 31. The prosthetic valve of any example herein, particularly example 30, wherein the plurality of outflow support arms comprises six outflow support arms.
Example 32. The prosthetic valve of any example herein, particularly any one of examples 29 to 31, wherein the at least one inflow support arm comprises a plurality of inflow support arms.
Example 33. The prosthetic valve of any example herein, particularly example 32, wherein the plurality of inflow support arms comprises six inflow support arms.
Example 34. The prosthetic valve of any example herein, particularly any one of examples 1 to 33, wherein the frame further comprises a plurality of cantilevered struts extending from the post inflow ends.
Example 35. The prosthetic valve of any example herein, particularly example 34, wherein the leaflets are coupled to the cantilevered struts.
Example 36. The prosthetic valve of any example herein, particularly example 27, wherein the leaflets are coupled to the inflow support arms.
Example 37. The prosthetic valve of any example herein, particularly any one of examples 1 to 36, further comprising an outer skirt mounted on the frame.
Example 38. The prosthetic valve of any example herein, particularly example 37, wherein, when depending on example 34, the outer skirt is coupled to the cantilevered struts.
Example 39. The prosthetic valve of any example herein, particularly example 37 or 38, wherein, when depending on example 27, the outer skirt is coupled to the inflow support arms.
Example 40. The prosthetic valve of any example herein, particularly example 37, wherein the skirt further comprises a sealing ring extending radially away from the frame.
Example 41. The prosthetic valve of any example herein, particularly example 40, wherein the sealing ring comprises a textured outer surface configured to encourage tissue overgrowth.
Example 42. The prosthetic valve of any example herein, particularly example 40 or 41, wherein the sealing ring is compressible.
Example 43. The prosthetic valve of any example herein, particularly example 40 or 42, wherein the sealing ring comprises a compressible insert and a cloth cover.
Example 44. The prosthetic valve of any example herein, particularly example 40 or 43, wherein the compressible insert comprises a silicone-based material.
Example 45. The prosthetic valve of any example herein, particularly example 40 or 42, wherein the sealing ring comprises a flat sheet of cloth rolled over itself to form a cylinder-like form.
Example 46. The prosthetic valve of any example herein, particularly any one of examples 40 to 45, wherein the sealing ring is disposed around an inflow end of the prosthetic valve.
Example 47. A prosthetic valve, comprising:
an annular frame movable between a radially compressed state and a radially expanded state, the frame comprising:
a plurality of support posts, wherein each support post extends between a post inflow end and an opposite post outflow end, and wherein the plurality of support posts comprises:
a plurality of commissure support posts, wherein each commissure support post comprises a commissure window; and
a plurality of non-commissural support posts, wherein each non-commissural support post is devoid of a commissure window;
a plurality of angled struts extending circumferentially between and interconnected with the support posts;
a valvular structure mounted within the frame and comprising a plurality of leaflets configured to regulate flow through the prosthetic valve; and
at least one support arm extending from the post outflow end or the post inflow end of at least one of the support posts, wherein the support arm terminates at a free-ended tip which is biased radially away from the corresponding support post;
wherein the width of the support post from which the support arm extends is greater than the width of any of the angled struts.
Example 48. The prosthetic valve of any example herein, particularly example 47, wherein the width of the support post is at least two times greater than the width of the angled struts.
Example 49. The prosthetic valve of any example herein, particularly example 47, wherein the width of the support post is at least three times greater than the width of the angled struts.
Example 50. The prosthetic valve of any example herein, particularly any one of examples 47 to 49, wherein the width of the support arm is less than the width of the support post it extends from.
Example 51. The prosthetic valve of any example herein, particularly example 50, wherein the width of the support arm is 70% or less than the width of the support post.
Example 52. The prosthetic valve of any example herein, particularly example 50, wherein the width of the support arm is 50% or less than the width of the support post.
Example 53. The prosthetic valve of any example herein, particularly example 50, wherein the width of the support arm is 30% or less than the width of the support post.
Example 54. The prosthetic valve of any example herein, particularly any one of examples 47 to 53, wherein two of the angled struts intersect with each post inflow end, and another two of the angled struts intersect with each post outflow end.
Example 55. The prosthetic valve of any example herein, particularly any one of examples 47 to 54, wherein each support post intersects with at least eight angled struts extending from adjacent actuation posts.
Example 56. The prosthetic valve of any example herein, particularly any one of examples 47 to 55, wherein the commissure support posts and the non-commissural support posts are arranged in an alternating manner along the circumference of the frame.
Example 57. The prosthetic valve of any example herein, particularly any one of examples 47 to 56, wherein the plurality of commissure support posts comprises three commissure support posts, and wherein the plurality of non-commissural support posts comprises three non-commissural support posts.
Example 58. The prosthetic valve of any example herein, particularly any one of examples 47 to 57, wherein the support arm is made of a shape-memory material.
Example 59. The prosthetic valve of any example herein, particularly example 58, wherein the shape-memory material comprises Nitinol.
Example 60. The prosthetic valve of any example herein, particularly any one of examples 47 to 59, wherein the support arm is circumferentially deflected relative to the frame, such that the tip is circumferentially offset from the corresponding support post.
Example 61. The prosthetic valve of any example herein, particularly any one of examples 47 to 60, wherein the support arm is twisted.
Example 62. The prosthetic valve of any example herein, particularly example 61, wherein the support arm comprises two twists.
Example 63. The prosthetic valve of any example herein, particularly any one of examples 47 to 62, wherein the tip is atraumatic.
Example 64. The prosthetic valve of any example herein, particularly example 62, wherein the tip is rounded.
Example 65. The prosthetic valve of any example herein, particularly any one of examples 47 to 64, wherein the support arm comprises a first arm portion extending from a base of the support arm at the end of the corresponding support posts, and a second arm portion continuously extending from the first arm portion to the tip.
Example 66. The prosthetic valve of any example herein, particularly example 65, wherein the first arm portion is C shaped.
Example 67. The prosthetic valve of any example herein, particularly example 65, wherein the first arm portion is L shaped.
Example 68. The prosthetic valve of any example herein, particularly any one of examples 47 to 67, wherein the at least one support arm comprises a plurality of support arms.
Example 69. The prosthetic valve of any example herein, particularly example 68, wherein the plurality of support arms comprises outflow support arms extending from the post outflow ends of corresponding support posts.
Example 70. The prosthetic valve of any example herein, particularly example 69, wherein all of the outflow support arms extend solely from non-commissural support posts.
Example 71. The prosthetic valve of any example herein, particularly example 69 or 70, wherein the plurality of outflow support arms comprises three outflow support arms.
Example 72. The prosthetic valve of any example herein, particularly example 68 or 69, wherein the plurality of support arms comprises inflow support arms extending from the post inflow ends of corresponding support posts.
Example 73. The prosthetic valve of any example herein, particularly example 72, wherein at least one of the inflow support arms extends from a commissure support post.
Example 74. The prosthetic valve of any example herein, particularly example 69, wherein the plurality of support arms comprises at least one outflow support arm extending from the post outflow end of at least one of the support posts, and at least one inflow support arm extending from the post inflow end of at least one of the support posts.
Example 75. The prosthetic valve of any example herein, particularly example 74, wherein the at least one outflow support arm comprises a plurality of outflow support arms.
Example 76. The prosthetic valve of any example herein, particularly example 74, wherein the plurality of outflow support arms comprises six outflow support arms.
Example 77. The prosthetic valve of any example herein, particularly any one of examples 74 to 76, wherein the at least one inflow support arm comprises a plurality of inflow support arms.
Example 78. The prosthetic valve of any example herein, particularly example 77, wherein the plurality of inflow support arms comprises six inflow support arms.
Example 79. The prosthetic valve of any example herein, particularly any one of examples 47 to 78, wherein the frame further comprises a plurality of cantilevered struts extending from the post inflow ends.
Example 80. The prosthetic valve of any example herein, particularly example 79, wherein the leaflets are coupled to the cantilevered struts.
Example 81. The prosthetic valve of any example herein, particularly example 72, wherein the leaflets are coupled to the inflow support arms.
Example 82. The prosthetic valve of any example herein, particularly any one of examples 47 to 81, further comprising an outer skirt mounted on the frame.
Example 83. The prosthetic valve of any example herein, particularly example 82, wherein, when depending on example 80, the outer skirt is coupled to the cantilevered struts.
Example 84. The prosthetic valve of any example herein, particularly example 82 or 83, wherein, when depending on example 72, the outer skirt is coupled to the inflow support arms.
Example 85. The prosthetic valve of any example herein, particularly example 82, wherein the skirt further comprises a sealing ring extending radially away from the frame.
Example 86. The prosthetic valve of any example herein, particularly example 85, wherein the sealing ring comprises a textured outer surface configured to encourage tissue overgrowth.
Example 87. The prosthetic valve of any example herein, particularly example 85 or 86, wherein the sealing ring is compressible.
Example 88. The prosthetic valve of any example herein, particularly example 87, wherein the sealing ring comprises a compressible insert and a cloth cover.
Example 89. The prosthetic valve of any example herein, particularly example 88, wherein the compressible insert comprises a silicone-based material.
Example 90. The prosthetic valve of any example herein, particularly example 87, wherein the sealing ring comprises a flat sheet of cloth rolled over itself to form a cylinder-like form.
Example 91. The prosthetic valve of any example herein, particularly any one of examples 85 to 90, wherein the sealing ring is disposed around an inflow end of the prosthetic valve.
Example 92. A delivery assembly, comprising:
a prosthetic valve comprising:
an annular frame movable between a radially compressed state and a radially expanded state, the frame comprising:
actuation posts comprising at least one upper post member and at least one lower post member;
a plurality of support posts, wherein each support post extends between a post inflow end and an opposite post outflow end, and wherein the plurality of support posts comprises:
a plurality of commissure support posts, wherein each commissure support post comprises a commissure window; and
a plurality of non-commissural support posts, wherein each non-commissural support post is devoid of a commissure window;
a plurality of angled struts extending circumferentially between adjacent actuation posts and support posts and interconnecting the actuation posts and the support posts; and
at least one actuator coupled to the actuation posts, the actuator configured to adjust the frame between the radially compressed state and the radially expanded state; and
at least one support arm extending from the post outflow end or the post inflow end of at least one of the support posts, wherein the support arm terminates at a free-ended tip which is biased radially away from the corresponding support post;
a delivery apparatus, comprising:
at least one actuator assembly releasably coupled to the actuator and configured to rotate the actuator to adjust the frame between the radially compressed state and the radially expanded state;
a handle comprising one or more control mechanisms, at least one of the one or more control mechanisms configured to be actuated to rotate the actuator assembly and the actuator of the prosthetic valve to adjust the frame between the radially compressed state and the radially expanded state; and
an outer shaft extending from the handle, wherein the actuator assembly is disposed within the outer shaft.
Example 93. The delivery assembly of any example herein, particularly example 92, wherein at least one of the one or more control mechanisms of the handle is configured to be actuated to decouple the actuator assembly from the actuator of the prosthetic valve.
Example 94. The delivery assembly of any example herein, particularly example 92 or 93, wherein the delivery apparatus further comprises a capsule coupled to a distal end of the outer shaft.
Example 95. The delivery assembly of any example herein, particularly example 94, wherein the capsule is configured to retain the prosthetic valve therein in the radially compressed state.
Example 96. The delivery assembly of any example herein, particularly example 95, wherein, when the prosthetic valve is retained in the compressed state within the capsule, the at least one support arm is straightened such that the tip is disposed proximal to outflow apices of the prosthetic valve.
Example 97. The delivery assembly of any example herein, particularly example 95, wherein, when the prosthetic valve is retained in the compressed state within the capsule, the at least one support arm is folded between the frame and the capsule such that the tip is disposed between inflow apices and outflow apices of the prosthetic valve.
Example 98. The delivery assembly of any example herein, particularly any one of examples 95 to 97, wherein at least one of the one or more control mechanisms of the handle is configured to be actuated to move the outer shaft axially relative to the prosthetic valve, to expose the prosthetic valve from the capsule.
Example 99. The delivery assembly of any example herein, particularly any one of examples 92 to 98, wherein the actuator comprises an actuator rod configured to be rotated in a first direction by the actuation assembly to radially expand the prosthetic valve and is configured to be rotated in an opposite second direction by the actuation assembly to radially compress the prosthetic valve.
Example 100. The delivery assembly of any example herein, particularly any one of examples 92 to 99, wherein the at least one actuator comprises a plurality of actuators, and wherein the at least one actuation assembly comprises a plurality of actuation assemblies.
Example 101. The delivery assembly of any example herein, particularly example 100, wherein the plurality of actuators comprises six actuators, and wherein the plurality of actuation assemblies comprises six actuation assemblies.
Example 102. The delivery assembly of any example herein, particularly any one of examples 92 to 101, wherein the prosthetic valve further comprises a valvular structure mounted within the frame and comprising a plurality of leaflets configured to regulate flow through the prosthetic valve.
Example 103. The delivery assembly of any example herein, particularly any one of examples 92 to 102, wherein two of the angled struts intersect with each post inflow end, and another two of the angled struts intersect with each post outflow end.
Example 104. The delivery assembly of any example herein, particularly any one of examples 92 to 103, wherein each support post intersects with at least eight angled struts extending from adjacent actuation posts.
Example 105. The delivery assembly of any example herein, particularly any one of examples 92 to 104, wherein the commissure support posts and the non-commissural support posts are arranged in an alternating manner along the circumference of the frame.
Example 106. The delivery assembly of any example herein, particularly any one of examples 92 to 105, wherein the plurality of commissure support posts comprises three commissure support posts, and wherein the plurality of non-commissural support posts comprises three non-commissural support posts.
Example 107. The delivery assembly of any example herein, particularly any one of examples 92 to 106, wherein the support arm is made of a shape-memory material.
Example 108. The delivery assembly of any example herein, particularly example 107, wherein the shape-memory material comprises Nitinol.
Example 109. The delivery assembly of any example herein, particularly any one of examples 92 to 108, wherein the width of the support post from which the support arm extends is greater than the width of any of the angled struts.
Example 110. The delivery assembly of any example herein, particularly example 109, wherein the width of the support post is at least two times greater than the width of the angled struts.
Example 111. The delivery assembly of any example herein, particularly example 109, wherein the width of the support post is at least three times greater than the width of the angled struts.
Example 112. The delivery assembly of any example herein, particularly any one of examples 92 to 111, wherein the width of the support arm is less than the width of the support post it extends from.
Example 113. The delivery assembly of any example herein, particularly example 112, wherein the width of the support arm is 70% or less than the width of the support post.
Example 114. The delivery assembly of any example herein, particularly example 112, wherein the width of the support arm is 50% or less than the width of the support post.
Example 115. The delivery assembly of any example herein, particularly example 112, wherein the width of the support arm is 30% or less than the width of the support post.
Example 116. The delivery assembly of any example herein, particularly any one of examples 92 to 115, wherein the support arm is circumferentially deflected relative to the frame, such that the tip is circumferentially offset from the corresponding support post.
Example 117. The delivery assembly of any example herein, particularly any one of examples 92 to 116, wherein the support arm is twisted.
Example 118. The delivery assembly of any example herein, particularly example 117, wherein the support arm comprises two twists.
Example 119. The delivery assembly of any example herein, particularly any one of examples 92 to 118, wherein the tip is atraumatic.
Example 120. The delivery assembly of any example herein, particularly example 119, wherein the tip is rounded.
Example 121. The delivery assembly of any example herein, particularly any one of examples 92 to 120, wherein the support arm comprises a first arm portion extending from a base of the support arm at the end of the corresponding support posts, and a second arm portion continuously extending from the first arm portion to the tip.
Example 122. The delivery assembly of any example herein, particularly example 121, wherein the first arm portion is C shaped.
Example 123. The delivery assembly of any example herein, particularly example 121, wherein the first arm portion is L shaped.
Example 124. The delivery assembly of any example herein, particularly any one of examples 92 to 123, wherein the at least one support arm comprises a plurality of support arms.
Example 125. The delivery assembly of any example herein, particularly example 124, wherein the plurality of support arms comprises outflow support arms extending from the post outflow ends of corresponding support posts.
Example 126. The delivery assembly of any example herein, particularly example 125, wherein all of the outflow support arms extend solely from non-commissural support posts.
Example 127. The delivery assembly of any example herein, particularly example 125 or 126, wherein the plurality of outflow support arms comprises three outflow support arms.
Example 128. The delivery assembly of any example herein, particularly example 124 or 125, wherein the plurality of support arms comprises inflow support arms extending from the post inflow ends of corresponding support posts.
Example 129. The delivery assembly of any example herein, particularly example 128, wherein at least one of the inflow support arms extends from a commissure support post.
Example 130. The delivery assembly of any example herein, particularly example 124, wherein the plurality of support arms comprises at least one outflow support arm extending from the post outflow end of at least one of the support posts, and at least one inflow support arm extending from the post inflow end of at least one of the support posts.
Example 131. The delivery assembly of any example herein, particularly example 130, wherein the at least one outflow support arm comprises a plurality of outflow support arms.
Example 132. The delivery assembly of any example herein, particularly example 131, wherein the plurality of outflow support arms comprises six outflow support arms.
Example 133. The delivery assembly of any example herein, particularly any one of examples 130 to 132, wherein the plurality of inflow support arms comprises six inflow support arms.
Example 134. The delivery assembly of any example herein, particularly example 133, wherein the frame further comprises a plurality of cantilevered struts extending from the post inflow ends.
Example 135. The delivery assembly of any example herein, particularly any one of examples 92 to 134, wherein
Example 136. The delivery assembly of any example herein, particularly example 135, further comprising leaflets coupled to the cantilevered struts.
Example 137. The delivery assembly of any example herein, particularly example 126, further comprising leaflets coupled to the inflow support arms.
Example 138. The delivery assembly of any example herein, particularly any one of examples 92 to 137, further comprising an outer skirt mounted on the frame.
Example 139. The delivery assembly of any example herein, particularly example 138, wherein, when depending on example 135, the outer skirt is coupled to the cantilevered struts.
Example 140. The delivery assembly of any example herein, particularly example 138 or 139, wherein, when depending on example 126, the outer skirt is coupled to the inflow support arms.
Example 141. The delivery assembly of any example herein, particularly example 138, wherein the skirt further comprises a sealing ring extending radially away from the frame. Example 142. A prosthetic valve, comprising:
an annular frame movable between a radially compressed state and a radially expanded state, the frame comprising:
a plurality of vertical posts comprising:
one or more pairs of actuation posts, each pair comprising an upper post member and a lower post member;
a plurality of support posts, wherein each support post extends between a post inflow end and an opposite post outflow end, wherein the plurality of support posts comprises:
a plurality of commissure support posts, wherein each commissure support post comprises a commissure window; and
a plurality of non-commissural support posts, wherein each non-commissural support post is devoid of a commissure window;
at least one post opening comprised in at least one of the vertical posts, the post opening extending between an opening outflow end and an opening inflow end;
a plurality of angled struts extending circumferentially between adjacent actuation posts and support posts and interconnecting the actuation posts and the support posts; and
one or more actuators coupled to the actuation posts, the one or more actuators configured to adjust the frame between the radially compressed state and the radially expanded state;
a valvular structure mounted within the frame and comprising a plurality of leaflets configured to regulate flow through the prosthetic valve; and
at least one support arm extending from the opening outflow end or the opening inflow end, wherein the support arm terminates at a free-ended tip which is biased radially away from the corresponding vertical post.
Example 143. The prosthetic valve of any example herein, particularly example 142, wherein the support arm is integrally formed with the corresponding vertical post it extends from.
Example 144. The prosthetic valve of any example herein, particularly example 142 or 143, wherein two of the angled struts intersect with each post inflow end, and another two of the angled struts intersect with each post outflow end.
Example 145. The prosthetic valve of any example herein, particularly any one of examples 142 to 144, wherein each support post intersects with at least eight angled struts extending from adjacent actuation posts.
Example 146. The prosthetic valve of any example herein, particularly any one of examples 142 to 145, wherein the commissure support posts and the non-commissural support posts are arranged in an alternating manner along the circumference of the frame.
Example 147. The prosthetic valve of any example herein, particularly any one of examples 142 to 146, wherein the plurality of commissure support posts comprises three commissure support posts, and wherein the plurality of non-commissural support posts comprises three non-commissural support posts.
Example 148. The prosthetic valve of any example herein, particularly any one of examples 142 to 147, wherein the support arm is made of a shape-memory material.
Example 149. The prosthetic valve of any example herein, particularly example 148, wherein the shape-memory material comprises Nitinol.
Example 150. The prosthetic valve of any example herein, particularly any one of examples 142 to 149, wherein the width of the vertical post from which the support arm extends is greater than the width of any of the angled struts.
Example 151. The prosthetic valve of any example herein, particularly example 150, wherein the width of the vertical post is at least two times greater than the width of the angled struts.
Example 152. The prosthetic valve of any example herein, particularly example 150, wherein the width of the vertical post is at least three times greater than the width of the angled struts.
Example 153. The prosthetic valve of any example herein, particularly any one of examples 150 to 152, wherein the width of the support arm is less than the width of the vertical post it extends from.
Example 154. The prosthetic valve of any example herein, particularly example 153, wherein the width of the support arm is 70% or less than the width of the vertical post.
Example 155. The prosthetic valve of any example herein, particularly example 153, wherein the width of the support arm is 50% or less than the width of the vertical post.
Example 156. The prosthetic valve of any example herein, particularly example 153, wherein the width of the support arm is 30% or less than the width of the vertical post.
Example 157. The prosthetic valve of any example herein, particularly any one of examples 150 to 152, wherein the width of the vertical post is greater than the width of the post opening comprised therein.
Example 158. The prosthetic valve of any example herein, particularly example 157, wherein the width of the vertical post is greater than the width of the post opening by at least 10%.
Example 159. The prosthetic valve of any example herein, particularly example 157, wherein the width of the vertical post is greater than the width of the post opening by at least 20%.
Example 160. The prosthetic valve of any example herein, particularly example 157, wherein the width of the vertical post is greater than the width of the post opening by at least 30%.
Example 161. The prosthetic valve of any example herein, particularly example 157, wherein the width of the vertical post is greater than the width of the post opening by at least 50%.
Example 162. The prosthetic valve of any example herein, particularly any one of examples 157 to 161, wherein the width of the vertical post is equal to or less than the width of the post opening.
Example 163. The prosthetic valve of any example herein, particularly any one of examples 142 to 162, wherein the tip is atraumatic.
Example 164. The prosthetic valve of any example herein, particularly example 163, wherein the tip is rounded.
Example 165. The prosthetic valve of any example herein, particularly any one of examples 142 to 164, wherein the at least one support arm extends from the opening outflow end of the post opening of at least one of the non-commissural support posts.
Example 166. The prosthetic valve of any example herein, particularly any one of examples 142 to 164, wherein the at least one support arm extends from the opening outflow end of the post opening of at least one of the upper post members.
Example 167. The prosthetic valve of any example herein, particularly any one of examples 142 to 164, wherein the at least one support arm comprises a plurality of support arms.
Example 168. The prosthetic valve of any example herein, particularly example 167, wherein the plurality of support arms comprises outflow support arms extending from the opening outflow ends of the post openings of corresponding non-commissural support posts.
Example 169. The prosthetic valve of any example herein, particularly example 168, wherein the plurality of outflow support arms comprises three outflow support arms.
Example 170. The prosthetic valve of any example herein, particularly example 167, wherein the plurality of support arms comprises inflow support arms extending from the opening inflow ends of corresponding post openings of the vertical posts.
Example 171. The prosthetic valve of any example herein, particularly example 170, wherein at least one of the inflow support arms extends from the opening inflow end of the post opening of at least one of the commissure support posts.
Example 172. The prosthetic valve of any example herein, particularly example 170, wherein at least one of the inflow support arms extends from the opening inflow end of the post opening of at least one of the lower post members.
Example 173. The prosthetic valve of any example herein, particularly example 167, wherein the plurality of support arms comprises at least one outflow support arm extending from the opening outflow end of the post opening of at least one of the non-commissural support posts or at least one of the upper post members, and at least one inflow support arm extending from the opening inflow end of the post opening of at least one of the vertical posts.
Example 174. The prosthetic valve of any example herein, particularly example 173, wherein the at least one outflow support arm comprises a plurality of outflow support arms.
Example 175. The prosthetic valve of any example herein, particularly example 174, wherein the plurality of outflow support arms comprises three outflow support arms.
Example 176. The prosthetic valve of any example herein, particularly example 174 or 175, wherein the at least one inflow support arm comprises a plurality of inflow support arms.
Example 177. The prosthetic valve of any example herein, particularly example 176, wherein the plurality of inflow support arms comprises at least six inflow support arms.
Example 178. The prosthetic valve of any example herein, particularly any one of examples 142 to 177, further comprising an outer skirt mounted on the frame.
Example 179. The prosthetic valve of any example herein, particularly example 178, wherein the skirt further comprises a sealing ring extending radially away from the frame.
Example 180. The prosthetic valve of any example herein, particularly example 179, wherein the sealing ring comprises a textured outer surface configured to encourage tissue overgrowth.
Example 181. The prosthetic valve of any example herein, particularly example 179 or 180, wherein the sealing ring is compressible.
Example 182. The prosthetic valve of any example herein, particularly example 181, wherein the sealing ring comprises a compressible insert and a cloth cover.
Example 183. The prosthetic valve of any example herein, particularly example 182, wherein the compressible insert comprises a silicone-based material.
Example 184. The prosthetic valve of any example herein, particularly example 181, wherein the sealing ring comprises a flat sheet of cloth rolled over itself to form a cylinder-like form.
Example 185. The prosthetic valve of any example herein, particularly any one of examples 181 to 184, wherein the sealing ring is disposed around an inflow end of the prosthetic valve.
Example 186. A prosthetic valve, comprising:
an annular frame movable between a radially compressed state and a radially expanded state, the frame comprising:
a plurality of support posts, wherein each support post extends between a post inflow end and an opposite post outflow end, wherein at least one of the support posts comprises a post opening extending between an opening outflow end which is distal to the post outflow end, and an opening inflow end which is proximal to the post inflow end, and wherein the plurality of support posts comprises:
a plurality of commissure support posts, wherein each commissure support post comprises a commissure window; and
a plurality of non-commissural support posts, wherein each non-commissural support post is devoid of a commissure window;
a plurality of angled struts extending circumferentially between and interconnected with the support posts;
a valvular structure mounted within the frame and comprising a plurality of leaflets configured to regulate flow through the prosthetic valve; and
at least one support arm extending from the opening outflow end or the opening inflow end, wherein the support arm terminates at a free-ended tip which is biased radially away from the corresponding support post;
wherein the width of the support post from which the support arm extends is greater than the width of any of the angled struts.
Example 187. The prosthetic valve of any example herein, particularly example 186, wherein the support arm is integrally formed with the corresponding support post it extends from.
Example 188. The prosthetic valve of any example herein, particularly example 186 or 187, wherein the width of the support post is at least two times greater than the width of the angled struts.
Example 189. The prosthetic valve of any example herein, particularly example 186 or 187, wherein the width of the support post is at least three times greater than the width of the angled struts.
Example 190. The prosthetic valve of any example herein, particularly any one of examples 186 to 189, wherein the width of the support arm is less than the width of the support post it extends from.
Example 191. The prosthetic valve of any example herein, particularly example 190, wherein the width of the support arm is 70% or less than the width of the support post.
Example 192. The prosthetic valve of any example herein, particularly example 190, wherein the width of the support arm is 50% or less than the width of the support post.
Example 193. The prosthetic valve of any example herein, particularly example 190, wherein the width of the support arm is 30% or less than the width of the support post.
Example 194. The prosthetic valve of any example herein, particularly any one of examples 186 to 189, wherein the width of the support post is greater than the width of the post opening comprised therein.
Example 195. The prosthetic valve of any example herein, particularly example 194, wherein the width of the support post is greater than the width of the post opening by at least 10%.
Example 196. The prosthetic valve of any example herein, particularly example 194, wherein the width of the support post is greater than the width of the post opening by at least 20%.
Example 197. The prosthetic valve of any example herein, particularly example 194, wherein the width of the support post is greater than the width of the post opening by at least 30%.
Example 198. The prosthetic valve of any example herein, particularly example 194, wherein the width of the support post is greater than the width of the post opening by at least 50%.
Example 199. The prosthetic valve of any example herein, particularly any one of examples 186 to 198, wherein two of the angled struts intersect with each post inflow end, and another two of the angled struts intersect with each post outflow end.
Example 200. The prosthetic valve of any example herein, particularly any one of examples 186 to 199, wherein each support post intersects with at least eight angled struts extending from adjacent actuation posts.
Example 201. The prosthetic valve of any example herein, particularly any one of examples 186 to 200, wherein the commissure support posts and the non-commissural support posts are arranged in an alternating manner along the circumference of the frame.
Example 202. The prosthetic valve of any example herein, particularly any one of examples 186 to 201, wherein the plurality of commissure support posts comprises three commissure support posts, and wherein the plurality of non-commissural support posts comprises three non-commissural support posts.
Example 203. The prosthetic valve of any example herein, particularly any one of examples 186 to 202, wherein the support arm is made of a shape-memory material.
Example 204. The prosthetic valve of any example herein, particularly example 203, wherein the shape-memory material comprises Nitinol.
Example 205. The prosthetic valve of any example herein, particularly any one of examples 186 to 204, wherein the tip is atraumatic.
Example 206. The prosthetic valve of any example herein, particularly example 205, wherein the tip is rounded.
Example 207. The prosthetic valve of any example herein, particularly any one of examples 186 to 206, wherein the at least one support arm extends from the opening outflow end of the post opening of at least one of the non-commissural support posts.
Example 208. The prosthetic valve of any example herein, particularly any one of examples 186 to 206, wherein the at least one support arm comprises a plurality of support arms.
Example 209. The prosthetic valve of any example herein, particularly example 208, wherein the at least one support arm comprises a plurality of support arms.
Example 210. The prosthetic valve of any example herein, particularly example 209, wherein the plurality of outflow support arms comprises three outflow support arms.
Example 211. The prosthetic valve of any example herein, particularly example 208, wherein the plurality of support arms comprises inflow support arms extending from the opening inflow ends of corresponding post openings of the support posts.
Example 212. The prosthetic valve of any example herein, particularly example 211, wherein at least one of the inflow support arms extends from the opening inflow end of the post opening of at least one of the commissure support posts.
Example 213. The prosthetic valve of any example herein, particularly example 208, wherein the plurality of support arms comprises at least one outflow support arm extending from the opening outflow end of the post opening of at least one of the non-commissural support posts, and at least one inflow support arm extending from the opening inflow end of the post opening of at least one of the support posts.
Example 214. The prosthetic valve of any example herein, particularly example 213, wherein the at least one outflow support arm comprises a plurality of outflow support arms.
Example 215. The prosthetic valve of any example herein, particularly example 214, wherein the plurality of outflow support arms comprises three outflow support arms.
Example 216. The prosthetic valve of any example herein, particularly example 214 or 215, wherein the at least one inflow support arm comprises a plurality of inflow support arms.
Example 217. The prosthetic valve of any example herein, particularly example 216, wherein the plurality of inflow support arms comprises six inflow support arms.
Example 218. The prosthetic valve of any example herein, particularly any one of examples 186 to 217, further comprising an outer skirt mounted on the frame.
Example 219. The prosthetic valve of any example herein, particularly example 218, wherein the skirt further comprises a scaling ring extending radially away from the frame.
Example 220. The prosthetic valve of any example herein, particularly example 219, wherein the sealing ring comprises a textured outer surface configured to encourage tissue overgrowth.
Example 221. The prosthetic valve of any example herein, particularly example 219 or 220, wherein the sealing ring is compressible.
Example 222. The prosthetic valve of any example herein, particularly example 221, wherein the sealing ring comprises a compressible insert and a cloth cover.
Example 223. The prosthetic valve of any example herein, particularly example 222, wherein the compressible insert comprises a silicone-based material.
Example 224. The prosthetic valve of any example herein, particularly example 221, wherein the sealing ring comprises a flat sheet of cloth rolled over itself to form a cylinder-like form.
Example 225. The prosthetic valve of any example herein, particularly any one of examples 221 to 224, wherein the sealing ring is disposed around an inflow end of the prosthetic valve.
Example 226. A delivery assembly, comprising:
a prosthetic valve comprising:
an annular frame movable between a radially compressed state and a radially expanded state, the frame comprising:
a plurality of vertical posts comprising:
actuation posts comprising at least one upper post member and at least one lower post member;
a plurality of support posts, wherein each support post extends between a post inflow end and an opposite post outflow end, wherein the plurality of support posts comprises:
a plurality of commissure support posts, wherein each commissure support post comprises a commissure window; and
a plurality of non-commissural support posts, wherein each non-commissural support post is devoid of a commissure window;
at least one post opening comprised in at least one of the vertical posts, the post opening extending between an opening outflow end and an opening inflow end;
a plurality of angled struts extending circumferentially between adjacent actuation posts and support posts and interconnecting the actuation posts and the support posts; and
at least one actuator coupled to the actuation posts, the actuator configured to adjust the frame between the radially compressed state and the radially expanded state; and
at least one support arm extending from the opening outflow end or the opening inflow end, wherein the support arm terminates at a free-ended tip which is biased radially away from the corresponding vertical post;
a delivery apparatus, comprising:
at least one actuator assembly releasably coupled to the actuator and configured to rotate the actuator to adjust the frame between the radially compressed state and the radially expanded state;
a handle comprising one or more control mechanisms, at least one of the one or more control mechanisms configured to be actuated to rotate the actuator assembly and the actuator of the prosthetic valve to adjust the frame between the radially compressed state and the radially expanded state; and
an outer shaft extending from the handle, wherein the actuator assembly is disposed within the outer shaft.
Example 227. The delivery assembly of any example herein, particularly example 226, wherein the support arm is integrally formed with the corresponding vertical post it extends from.
Example 228. The delivery assembly of any example herein, particularly example 226 or 227, at least one of the one or more control mechanisms of the handle is configured to be actuated to decouple the actuator assembly from the actuator of the prosthetic valve.
Example 229. The delivery assembly of any example herein, particularly any one of examples 226 to 228, wherein the delivery apparatus further comprises a capsule coupled to a distal end of the outer shaft.
Example 230. The delivery assembly of any example herein, particularly example 229, wherein the capsule is configured to retain the prosthetic valve therein in the radially compressed state.
Example 231. The delivery assembly of any example herein, particularly example 230, wherein, when the prosthetic valve is retained in the compressed state within the capsule, the at least one support arm is positioned in the respective post opening.
Example 232. The delivery assembly of any example herein, particularly example 231, wherein the support arm is flush with an outer surface of the support post.
Example 233. The delivery assembly of any example herein, particularly any one of examples 230 to 232, wherein at least one of the one or more control mechanisms of the handle is configured to be actuated to move the outer shaft axially relative to the prosthetic valve, to expose the prosthetic valve from the capsule.
Example 234. The delivery assembly of any example herein, particularly any one of examples 226 to 233, wherein the actuator comprises an actuator rod configured to be rotated in a first direction by the actuation assembly to radially expand the prosthetic valve and is configured to be rotated in an opposite second direction by the actuation assembly to radially compress the prosthetic valve.
Example 235. The delivery assembly of any example herein, particularly any one of examples 226 to 234, wherein the at least one actuator comprises a plurality of actuators, and wherein the at least one actuation assembly comprises a plurality of actuation assemblies.
Example 236. The delivery assembly of any example herein, particularly example 235, wherein the plurality of actuators comprises six actuators, and wherein the plurality of actuation assemblies comprises six actuation assemblies.
Example 237. The delivery assembly of any example herein, particularly any one of examples 226 to 236, wherein the prosthetic valve further comprises a valvular structure mounted within the frame and comprising a plurality of leaflets configured to regulate flow through the prosthetic valve.
Example 238. The delivery assembly of any example herein, particularly any one of examples 226 to 237, wherein two of the angled struts intersect with each post inflow end, and another two of the angled struts intersect with each post outflow end.
Example 239. The delivery assembly of any example herein, particularly any one of examples 226 to 238, wherein each support post intersects with at least eight angled struts extending from adjacent actuation posts.
Example 240. The delivery assembly of any example herein, particularly any one of examples 226 to 239, wherein the commissure support posts and the non-commissural support posts are arranged in an alternating manner along the circumference of the frame.
Example 241. The delivery assembly of any example herein, particularly any one of examples 226 to 240, wherein the plurality of commissure support posts comprises three commissure support posts, and wherein the plurality of non-commissural support posts comprises three non-commissural support posts.
Example 242. The delivery assembly of any example herein, particularly any one of examples 226 to 241, wherein the support arm is made of a shape-memory material.
Example 243. The delivery assembly of any example herein, particularly example 242, wherein the shape-memory material comprises Nitinol.
Example 244. The delivery assembly of any example herein, particularly any one of examples 226 to 243, wherein the width of the vertical post from which the support arm extends is greater than the width of any of the angled struts.
Example 245. The delivery assembly of any example herein, particularly example 244, wherein the width of the vertical post is at least two times greater than the width of the angled struts.
Example 246. The delivery assembly of any example herein, particularly example 244, wherein the width of the vertical post is at least three times greater than the width of the angled struts.
Example 247. The delivery assembly of any example herein, particularly any one of examples 244 to 246, wherein the width of the support arm is less than the width of the vertical post it extends from.
Example 248. The delivery assembly of any example herein, particularly example 247, wherein the width of the support arm is 70% or less than the width of the support post.
Example 249. The delivery assembly of any example herein, particularly example 247, wherein the width of the support arm is 50% or less than the width of the support post.
Example 250. The delivery assembly of any example herein, particularly example 247, wherein the width of the support arm is 30% or less than the width of the support post.
Example 251. The delivery assembly of any example herein, particularly any one of examples 244 to 246, wherein the width of the vertical post is greater than the width of the post opening comprised therein.
Example 252. The delivery assembly of any example herein, particularly example 251, wherein the width of the vertical post is greater than the width of the post opening by at least 10%.
Example 253. The delivery assembly of any example herein, particularly example 251, wherein the width of the vertical post is greater than the width of the post opening by at least 20%.
Example 254. The delivery assembly of any example herein, particularly example 251, wherein the width of the vertical post is greater than the width of the post opening by at least 30%.
Example 255. The delivery assembly of any example herein, particularly example 251, wherein the width of the vertical post is greater than the width of the post opening by at least 50%.
Example 256. The delivery assembly of any example herein, particularly any one of examples 226 to 255, wherein the tip is atraumatic.
Example 257. The delivery assembly of any example herein, particularly example 256, wherein the tip is rounded.
Example 258. The delivery assembly of any example herein, particularly any one of examples 226 to 257, wherein the at least one support arm extends from the opening outflow end of the post opening of at least one of the non-commissural support posts.
Example 259. The delivery assembly of any example herein, particularly any one of examples 226 to 257, wherein the at least one support arm extends from the opening outflow end of the post opening of at least one of the upper post members.
Example 260. The delivery assembly of any example herein, particularly any one of examples 226 to 257, wherein the at least one support arm comprises a plurality of support arms.
Example 261. The delivery assembly of any example herein, particularly example 260, wherein the plurality of support arms comprises outflow support arms extending from the opening outflow ends of the post openings of corresponding non-commissural support posts.
Example 262. The delivery assembly of any example herein, particularly example 261, wherein the plurality of outflow support arms comprises three outflow support arms.
Example 263. The delivery assembly of any example herein, particularly example 261, wherein the plurality of support arms comprises inflow support arms extending from the opening inflow ends of corresponding post openings of the support posts.
Example 264. The delivery assembly of any example herein, particularly example 263, wherein at least one of the inflow support arms extends from the opening inflow end of the post opening of at least one of the commissure support posts.
Example 265. The delivery assembly of any example herein, particularly example 263, wherein at least one of the inflow support arms extends from the opening inflow end of the post opening of at least one of the lower post members.
Example 266. The delivery assembly of any example herein, particularly example 261, wherein the plurality of support arms comprises at least one outflow support arm extending from the opening outflow end of the post opening of at least one of the non-commissural support posts or at least one of the upper post members, and at least one inflow support arm extending from the opening inflow end of the post opening of at least one of the vertical posts.
Example 267. The delivery assembly of any example herein, particularly example 266, wherein the at least one outflow support arm comprises a plurality of outflow support arms.
Example 268. The delivery assembly of any example herein, particularly example 267, wherein the plurality of outflow support arms comprises three outflow support arms.
Example 269. The delivery assembly of any example herein, particularly example 267 or 268, wherein the at least one inflow support arm comprises a plurality of inflow support arms.
Example 270. The delivery assembly of any example herein, particularly example 269, wherein the plurality of inflow support arms comprises at least six inflow support arms.
Example 271. The delivery assembly of any example herein, particularly any one of examples 226 to 270, further comprising an outer skirt mounted on the frame.
Example 272. The delivery assembly of any example herein, particularly example 271, wherein the skirt further comprises a sealing ring extending radially away from the frame.
Example 273. The delivery assembly of any example herein, particularly example 272, wherein the sealing ring comprises a textured outer surface configured to encourage tissue overgrowth.
Example 274. The delivery assembly of any example herein, particularly example 272 or 273, wherein the sealing ring is compressible.
Example 275. The delivery assembly of any example herein, particularly example 274, wherein the sealing ring comprises a compressible insert and a cloth cover.
Example 276. The delivery assembly of any example herein, particularly example 275, wherein the compressible insert comprises a silicone-based material.
Example 277. The delivery assembly of any example herein, particularly example 274, wherein the sealing ring comprises a flat sheet of cloth rolled over itself to form a cylinder-like form.
Example 278. The delivery assembly of any example herein, particularly any one of examples 274 to 277, the sealing ring is disposed around an inflow end of the prosthetic valve.
Example 279. A prosthetic assembly, comprising:
a docking station comprising:
a radially collapsible and expandable docking frame comprising an inflow end portion and an outflow end portion;
a sealing member disposed on the outflow end portion and configured to form a seal between the docking station and a body lumen, the sealing member defining a sealing member inflow end and a sealing member outflow end; and
a valve seat extending from the outflow end portion and defining a diameter which is less than the diameter of a main body of the docking frame;
a prosthetic valve comprising:
an annular frame movable between a radially compressed state and a radially expanded state, the frame comprising:
a plurality of vertical posts comprising a plurality of support posts, wherein each support post extends between a post inflow end and an opposite post outflow end, and wherein the plurality of support posts comprises:
a plurality of commissure support posts, wherein each commissure support post comprises a commissure window; and
a plurality of non-commissural support posts, wherein each non-commissural support post is devoid of a commissure window;
a plurality of angled struts extending circumferentially between and interconnected with the support posts;
a valvular structure mounted within the frame and comprising a plurality of leaflets configured to regulate flow through the prosthetic valve; and
at least one support arm extending from at least one of the vertical posts, wherein the support arm terminates at a free-ended tip which is biased radially away from the corresponding vertical post, defining a radial gap between the tip and the vertical post;
wherein the sealing member is retained between the annular frame of the prosthetic valve, in the radially expanded state, and the at least one support arm, such that the annular frame is positioned radially inward to the sealing member, and the tip is positioned radially outward to the sealing member.
Example 280. The prosthetic assembly of any example herein, particularly example 279, wherein the docking frame comprises a plurality of longitudinal strut members circumferentially spaced apart from each other.
Example 281. The prosthetic assembly of any example herein, particularly example 280, wherein the docking frame further comprises a plurality of angled struts extending between adjacent longitudinal struts.
Example 282. The prosthetic assembly of any example herein, particularly any one of examples 279 to 281, wherein the support arm is integrally formed with the vertical post it extends from.
Example 283. The prosthetic assembly of any example herein, particularly any one of examples 279 to 282, wherein the commissure support posts and the non-commissural support posts are arranged in an alternating manner along the circumference of the frame.
Example 284. The prosthetic assembly of any example herein, particularly any one of examples 279 to 283, wherein the plurality of commissure support posts comprises three commissure support posts, and wherein the plurality of non-commissural support posts comprises three non-commissural support posts.
Example 285. The prosthetic assembly of any example herein, particularly any one of examples 279 to 284, wherein the support arm is made of a shape-memory material.
Example 286. The prosthetic assembly of any example herein, particularly example 285, wherein the shape-memory material comprises Nitinol.
Example 287. The prosthetic assembly of any example herein, particularly any one of examples 279 to 286, wherein the width of the vertical post from which the support arm extends is greater than the width of any of the angled struts.
Example 288. The prosthetic assembly of any example herein, particularly example 285, wherein the width of the vertical post is at least two times greater than the width of the angled struts.
Example 289. The prosthetic assembly of any example herein, particularly example 285, wherein the width of the vertical post is at least three times greater than the width of the angled struts.
Example 290. The prosthetic assembly of any example herein, particularly any one of examples 287 to 289, wherein the width of the support arm is less than the width of the vertical post it extends from.
Example 291. The prosthetic assembly of any example herein, particularly example 290, wherein the width of the support arm is 70% or less than the width of the vertical post.
Example 292. The prosthetic assembly of any example herein, particularly example 290, wherein the width of the support arm is 50% or less than the width of the vertical post.
Example 293. The prosthetic assembly of any example herein, particularly example 290, wherein the width of the support arm is 30% or less than the width of the vertical post.
Example 294. The prosthetic assembly of any example herein, particularly any one of examples 279 to 293, wherein the tip is atraumatic.
Example 295. The prosthetic assembly of any example herein, particularly example 294, wherein the tip is rounded.
Example 296. The prosthetic assembly of any example herein, particularly any one of examples 279 to 295, wherein the at least one support arm comprises at least one outflow support arm extending from the post outflow end of the corresponding support post.
Example 297. The prosthetic assembly of any example herein, particularly example 296, wherein the outflow support arm abuts the sealing member outflow end.
Example 298. The prosthetic assembly of any example herein, particularly example 296 or 297, wherein the at least one outflow support arm comprises a plurality of outflow support arms.
Example 299. The prosthetic assembly of any example herein, particularly any one of examples 279 to 298, wherein the at least one support arm further comprises at least one inflow support arm extending from the post inflow end of the corresponding support post.
Example 300. The prosthetic assembly of any example herein, particularly example 299, wherein the inflow support arm abuts the sealing member inflow end.
Example 301. The prosthetic assembly of any example herein, particularly example 299 or 300, wherein the at least one inflow support arm comprises a plurality of inflow support arms.
Example 302. The prosthetic assembly of any example herein, particularly any one of examples 299 to 301, wherein the radial gap defined by the at least one outflow support arm is less than the radial gap defined by the at least one inflow support arm.
Example 303. The prosthetic assembly of any example herein, particularly any one of examples 279 to 295, wherein the at least one vertical post from which the support arm extends further comprises a post opening extending between an opening outflow end and an opening inflow end.
Example 304. The prosthetic assembly of any example herein, particularly example 303, wherein the vertical posts further comprise actuation posts comprising at least one upper post member and at least one lower post member.
Example 305. The prosthetic assembly of any example herein, particularly example 303 or 304, wherein the at least one support arm comprises at least one outflow support arm extending from the opening outflow end.
Example 306. The prosthetic assembly of any example herein, particularly example 305, wherein the outflow support arm abuts the sealing member outflow end of the corresponding post opening.
Example 307. The prosthetic assembly of any example herein, particularly example 305 or 306, wherein the at least one outflow support arm comprises a plurality of outflow support arms.
Example 308. The prosthetic assembly of any example herein, particularly any one of examples 305 to 307, wherein the at least one support arm further comprises at least one inflow support arm extending from the opening inflow end of the corresponding post opening.
Example 309. The prosthetic assembly of any example herein, particularly example 308, wherein the inflow support arm abuts the scaling member inflow end.
Example 310. The prosthetic assembly of any example herein, particularly example 308 or 309, wherein the at least one inflow support arm comprises a plurality of inflow support arms.
Example 311. The prosthetic assembly of any example herein, particularly any one of examples 308 to 309, wherein the radial gap defined by the at least one outflow support arm is less than the radial gap defined by the at least one inflow support arm.
Example 312. A method, comprising:
advancing a prosthetic valve in a radially compressed state toward a native heart valve, the prosthetic valve comprising:
an annular frame movable between a radially compressed state and a radially expanded state, the frame comprising:
a plurality of vertical posts comprising a plurality of support posts, wherein each support post extends between a post inflow end and an opposite post outflow end, and wherein the plurality of support posts comprises:
a plurality of commissure support posts, wherein each commissure support post comprises a commissure window; and
a plurality of non-commissural support posts, wherein each non-commissural support post is devoid of a commissure window;
a plurality of angled struts extending circumferentially between and interconnected with the support posts; and
at least one support arm extending from at least one of the vertical posts, wherein the support arm terminates at a free-ended tip which is biased radially away from the corresponding vertical post;
partially expanding the prosthetic valve at a position in which the outflow end of the prosthetic valve is proximal to native leaflets of the native heart valve;
angularly orienting the prosthetic valve such that the commissures of the prosthetic valve are aligned with native commissures of the native leaflets; and
further expanding the prosthetic valve within a native annulus of the native heart valve.
Example 313. The method of any example herein, particularly example 312, wherein the support arm is integrally formed with the vertical post it extends from.
Example 314. The method of any example herein, particularly example 312 or 313, wherein the vertical posts further comprise actuation posts comprising at least one upper post member and at least one lower post member, and wherein the prosthetic valve further comprises at least one actuator coupled to the actuation posts.
Example 315. The method of any example herein, particularly example 314, wherein expanding the prosthetic valve comprises rotating the at least one actuator in a direction that approximates between the upper post member and the lower post member.
Example 316. The method of any example herein, particularly example 314 or 315, wherein the at least one actuator comprises a plurality of actuators.
Example 317. The method of any example herein, particularly example 316, wherein the plurality of actuators comprises six actuators.
Example 318. The method of any example herein, particularly any one of examples 312 to 317, wherein two of the angled struts intersect with each post inflow end, and another two of the angled struts intersect with each post outflow end.
Example 319. The method of any example herein, particularly any one of examples 314 to 317, wherein each support post intersects with at least eight angled struts extending from adjacent actuation posts.
Example 320. The method of any example herein, particularly any one of examples 312 to 319, wherein the commissure support posts and the non-commissural support posts are arranged in an alternating manner along the circumference of the frame.
Example 321. The method of any example herein, particularly any one of examples 312 to 320, wherein the plurality of commissure support posts comprises three commissure support posts, and wherein the plurality of non-commissural support posts comprises three non-commissural support posts.
Example 322. The method of any example herein, particularly any one of examples 312 to 321, wherein the support arm is made of a shape-memory material.
Example 323. The method of any example herein, particularly example 322, wherein the shape-memory material comprises Nitinol.
Example 324. The method of any example herein, particularly any one of examples 312 to 323, wherein the prosthetic valve further comprises a valvular structure mounted within the frame and comprising a plurality of leaflets configured to regulate flow through the prosthetic valve.
Example 325. The method of any example herein, particularly any one of examples 312 to 324, wherein the width of the vertical post from which the support arm extends is greater than the width of any of the angled struts.
Example 326. The method of any example herein, particularly example 325, wherein the width of the vertical post is at least two times greater than the width of the angled struts.
Example 327. The method of any example herein, particularly example 325, wherein the width of the vertical post is at least three times greater than the width of the angled struts.
Example 328. The method of any example herein, particularly any one of examples 325 to 327, wherein the width of the support arm is less than the width of the vertical post it extends from.
Example 329. The method of any example herein, particularly example 328, wherein the width of the support arm is 70% or less than the width of the vertical post.
Example 330. The method of any example herein, particularly example 328, wherein the width of the support arm is 50% or less than the width of the vertical post.
Example 331. The method of any example herein, particularly example 328, wherein the width of the support arm is 30% or less than the width of the vertical post.
Example 332. The method of any example herein, particularly any one of examples 312 to 331, wherein the tip is atraumatic.
Example 333. The method of any example herein, particularly example 332, wherein the tip is rounded.
Example 334. The method of any example herein, particularly any one of examples 312 to 333, further comprising distally advancing the prosthetic valve, after partially expanding it and prior to fully expanding it, until the tips contact an abutment surface of the native heart valve.
Example 335. The method of any example herein, particularly any one of examples 312 to 334, wherein fully expanding the prosthetic valve comprises clamping native leaflets of the native heart valve between the frame and the at least one support arm.
Example 336. The method of any example herein, particularly any one of examples 312 to 335, wherein the at least one support arm comprises at least one outflow support arm extending from the post outflow end of the corresponding support post.
Example 337. The method of any example herein, particularly any one of examples 312 to 335, wherein the at least one support arm comprises at least one outflow support arm extending from an opening outflow end of a post opening formed in a corresponding non-commissural post of the plurality of support post.
Example 338. The method of any example herein, particularly any one of examples 312 to 335, wherein, when depending on example 314, the at least one support arm comprises at least one outflow support arm extending from an opening outflow end of a post opening formed in a corresponding upper post member of the plurality of actuation posts.
Example 339. The method of any example herein, particularly any one of examples 336 to 338, wherein the at least one outflow support arm comprises a plurality of outflow support arms.
Example 340. The method of any example herein, particularly example 339, wherein the plurality of outflow support arms comprises three outflow support arms.
Example 341. The method of any example herein, particularly any one of examples 312 to 340, wherein advancing a prosthetic valve in a radially compressed state comprises retaining the prosthetic valve in a capsule of a delivery apparatus coupled to the prosthetic valve.
Example 342. The method of any example herein, particularly example 336, wherein, when depending on example 336, retaining the prosthetic valve in the capsule comprises keeping the support arm in a straightened configuration, such that the tip is disposed proximal to outflow apices of the prosthetic valve.
Example 343. The method of any example herein, particularly example 336, wherein, when depending on example 336, retaining the prosthetic valve in the capsule comprises keeping the support arm in a folded configuration between the frame and the capsule, such that the tip is disposed between inflow apices and outflow apices of the prosthetic valve.
Example 344. The method of any example herein, particularly example 337 or 338, wherein, when depending on example 337 or 338, retaining the prosthetic valve in the capsule comprises keeping the support arm positioned in the respective post opening.
Example 345. The method of any example herein, particularly example 344, wherein the support arm is flush with an outer surface of the vertical post.
Example 346. The method of any example herein, particularly any one of examples 341 to 345, further comprising moving the capsule axially relative to the prosthetic valve to expose the prosthetic valve from the capsule, prior to partially expanding the prosthetic valve.
Example 347. The method of any example herein, particularly any one of examples 312 to 346, wherein the native heart valve is a native aortic valve.

It is appreciated that certain features of the disclosed technology, which are, for clarity, described in the context of separate examples, may also be provided in combination in a single example. Conversely, various features of the disclosed technology, which are, for brevity, described in the context of a single example, may also be provided separately or in any suitable sub-combination or as suitable in any other described example of the disclosed technology. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A prosthetic valve comprising:
an annular frame movable between a radially compressed state and a radially expanded state, the frame comprising:
a plurality of support posts, wherein each support post extends between a post inflow end and an opposite post outflow end, and wherein the plurality of support posts comprises:
a plurality of commissure support posts, wherein each commissure support post comprises a commissure window; and
a plurality of non-commissural support posts, wherein each non-commissural support post is devoid of a commissure window;
a valvular structure mounted within the frame and comprising a plurality of leaflets configured to regulate flow through the prosthetic valve; and
at least one support arm extending from the post outflow end or the post inflow end of at least one of the support posts, wherein the support arm terminates at a free-ended tip which is biased radially away from the corresponding support post.

2. The prosthetic valve of claim 1, wherein the support arm is circumferentially deflected relative to the frame, such that the tip is circumferentially offset from the corresponding support post.

3. The prosthetic valve of claim 1, wherein the support arm is twisted.

4. The prosthetic valve of claim 1, wherein the support arm comprises a first arm portion extending from a base of the support arm at the end of the corresponding support posts, and a second arm portion continuously extending from the first arm portion to the tip.

5. The prosthetic valve of claim 4, wherein the plurality of support arms comprises at least one outflow support arm extending from the post outflow end of at least one of the support posts, and at least one inflow support arm extending from the post inflow end of at least one of the support posts.

6. The prosthetic valve of claim 1, wherein the frame comprises a plurality of angled struts extending circumferentially between the support posts.

7. The prosthetic valve of claim 6, wherein the width of the support post from which the support arm extends is greater than the width of any of the angled struts.

8. The prosthetic valve of claim 1, further comprising an outer skirt mounted on the frame.

9. The prosthetic valve of claim 8, wherein the skirt further comprises a sealing ring extending radially away from the frame.

10. The prosthetic valve of claim 9, wherein the sealing ring is compressible.

11. A prosthetic valve comprising:
an annular frame movable between a radially compressed state and a radially expanded state, the frame comprising:
a plurality of support posts, wherein each support post extends between a post inflow end and an opposite post outflow end, wherein at least one of the support posts comprises a post opening extending between an opening outflow end which is distal to the post outflow end, and an opening inflow end which is proximal to the post inflow end, and wherein the plurality of support posts comprises:
a plurality of commissure support posts, wherein each commissure support post comprises a commissure window; and
a plurality of non-commissural support posts, wherein each non-commissural support post is devoid of a commissure window;
a valvular structure mounted within the frame and comprising a plurality of leaflets configured to regulate flow through the prosthetic valve; and
at least one support arm extending from the opening outflow end or the opening inflow end, wherein the support arm terminates at a free-ended tip which is biased radially away from the corresponding support post.

12. The prosthetic valve of claim 11, wherein the support arm is integrally formed with the corresponding support post it extends from.

13. The prosthetic valve of claim 11, wherein the at least one support arm comprises a plurality of support arms.

14. The prosthetic valve of claim 13, wherein the plurality of support arms comprises inflow support arms extending from the opening inflow ends of corresponding post openings of the support posts.

15. The prosthetic valve of claim 13, wherein the plurality of support arms comprises at least one outflow support arm extending from the opening outflow end of the post opening of at least one of the non-commissural support posts, and at least one inflow support arm extending from the opening inflow end of the post opening of at least one of the support posts.

16. A method comprising:
advancing a prosthetic valve in a radially compressed state toward a native heart valve, the prosthetic valve comprising:
an annular frame movable between a radially compressed state and a radially expanded state, the frame comprising:
a plurality of vertical posts comprising a plurality of support posts, wherein each support post extends between a post inflow end and an opposite post outflow end, and wherein the plurality of support posts comprises:
a plurality of commissure support posts, wherein each commissure support post comprises a commissure window; and
a plurality of non-commissural support posts, wherein each non-commissural support post is devoid of a commissure window; and
at least one support arm extending from at least one of the vertical posts, wherein the support arm terminates at a free-ended tip which is biased radially away from the corresponding vertical post;
partially expanding the prosthetic valve at a position in which the outflow end of the prosthetic valve is proximal to native leaflets of the native heart valve;
angularly orienting the prosthetic valve such that commissures of the prosthetic valve are aligned with native commissures of the native leaflets; and
further expanding the prosthetic valve within a native annulus of the native heart valve.

17. The method of claim 16, further comprising distally advancing the prosthetic valve, after partially expanding it and prior to fully expanding it, until the tips contact an abutment surface of the native heart valve.

18. The method of claim 16, wherein fully expanding the prosthetic valve comprises clamping native leaflets of the native heart valve between the frame and the at least one support arm.

19. The method of claim 16, wherein advancing a prosthetic valve in a radially compressed state comprises retaining the prosthetic valve in a capsule of a delivery apparatus coupled to the prosthetic valve.

20. The method of claim 19, further comprising moving the capsule axially relative to the prosthetic valve to expose the prosthetic valve from the capsule, prior to partially expanding the prosthetic valve.
